SEQUENCE LISTING (18 sequences)

SEQ ID NO: 1 - DNA, 215 bp, Human alphaherpesvirus 1
  source 1..215: mol_type = genomic DNA
ggccagaaccgccgtgcacgacccggagcgtcccctgctgcgctctcccgggctgctgcccgaaatcgcccccaacgcatccttgggtgtggcacatcgaagaaccggcgggaccgtgaccgacagtccccgtaatccggtaacccgttgagtcccgggtacgaccatcacccgagtctctgggcggagggtggttcccccccgtgtctctcgag

SEQ ID NO: 2 - DNA, 50 bp, Human alphaherpesvirus 1
  source 1..50: mol_type = genomic DNA
gccgacgtacgcgatgagatcaataaaagggggcgtgaggaccgggaggc

SEQ ID NO: 3 - DNA, 50 bp, Human alphaherpesvirus 1
  source 1..50: mol_type = genomic DNA
atgagccagacccaacccccggccccagttgggccgggcgacccagatgt

SEQ ID NO: 4 - RNA, 215 nt, Human alphaherpesvirus 1
  source 1..215: mol_type = mRNA
ggccagaaccgccgtgcacgacccggagcgtcccctgctgcgctctcccgggctgctgcccgaaatcgcccccaacgcatccttgggtgtggcacatcgaagaaccggcgggaccgtgaccgacagtccccgtaatccggtaacccgttgagtcccgggtacgaccatcacccgagtctctgggcggagggtggttcccccccgtgtctctcgag

SEQ ID NO: 5 - RNA, 215 nt, Human alphaherpesvirus 1; identical to SEQ ID NO: 4
  source 1..215: mol_type = mRNA

SEQ ID NO: 6 - RNA, 50 nt, Human alphaherpesvirus 1
  source 1..50: mol_type = mRNA
atgagccagacccaacccccggccccagttgggccgggcgacccagatgt

SEQ ID NO: 7 - DNA, 286 bp, Human alphaherpesvirus 1
  source 1..286: mol_type = genomic DNA
acactctttgcctcggtctaccggtgcggggagctcgagttgcgccgcccggactgcagccgcccgacctccgaaggtcgttaccgttacccgcccggcgtatatctcacgtacgactccgactgtccgctggtggccatcgtcgagagcgcccccgacggctgtatcggcccccggtcggtcgtggtctacgaccgagacgttttctcgatcctctactcggtcctccagcacctcgcccccaggctacctgacggggggcacgacgggcccccgtagtcccgcc

SEQ ID NO: 8 - DNA, 50 bp, Human alphaherpesvirus 1
  source 1..50: mol_type = genomic DNA
ccactcagcgcgccgcctggcgatatattcgcgagctgattatcgccacc

SEQ ID NO: 9 - DNA, 50 bp, Human alphaherpesvirus 1
  source 1..50: mol_type = genomic DNA
atgcaccagggcgccccctcgtgggggcgccggtggttcgtcgtatgggc

SEQ ID NO: 10 - RNA, 286 nt, Human alphaherpesvirus 1
  source 1..286: mol_type = mRNA
acactctttgcctcggtctaccggtgcggggagctcgagttgcgccgcccggactgcagccgcccgacctccgaaggtcgttaccgttacccgcccggcgtatatctcacgtacgactccgactgtccgctggtggccatcgtcgagagcgcccccgacggctgtatcggcccccggtcggtcgtggtctacgaccgcgacgttttctcgatcctctactcggtcctccagcacctcgcccccaggctacctgacggggggcacgacgggcccccgtagtcccgcc

SEQ ID NO: 11 - RNA, 50 nt, Human alphaherpesvirus 1
  source 1..50: mol_type = mRNA
ccactcagcgcgccgcctggcgatatattcgcgagctgattatcgccacc

SEQ ID NO: 12 - RNA, 50 nt, Human alphaherpesvirus 1
  source 1..50: mol_type = mRNA
atgcaccagggcgccccctcgtgggggcgccggtggttcgtcgtatgggc

SEQ ID NO: 13 - DNA, 239 bp, Human alphaherpesvirus 1
  source 1..239: mol_type = genomic DNA
ggtctgttggggacactgggttctcctggaacgaggccgcagccttctcccggtgcctttcccccccgaccgacacccggcctctcacacagcatcccccgcctttttgggtccgggcccgtcgtgtctttcggtggaccttgggccgtcgggcacgtacacgggtggccgggcgttggggtggatcttagcctccccgggccaatatcgctagagacagccgatctccacgcgacccc

SEQ ID NO: 14 - DNA, 50 bp, Human alphaherpesvirus 1
  source 1..50: mol_type = genomic DNA
acgggggtggggcggggggggtatataaggcctgggatcccacgtccccg

SEQ ID NO: 15 - DNA, 50 bp, Human alphaherpesvirus 1
  source 1..50: mol_type = genomic DNA
atggccgctcccaaccgcgaccctccgggataccggtatgccgcggccat

SEQ ID NO: 16 - RNA, 239 nt, Human alphaherpesvirus 1
  source 1..239: mol_type = mRNA
ggtctgttggggacactgggttctcctggaacgaggccgcagccttctcccggtgcctttcccccccgaccgacacccggcctctcacacagcatcccccgcctttttgggtccgggcccgtcgtgtctttcggtggaccttgggccgtcgggcacgtacacgggtggccgggcgttggggtggatcttagcctccccgggccaatatcgctagagacagccgatctccacgcgacccc

SEQ ID NO: 17 - RNA, 50 nt, Human alphaherpesvirus 1
  source 1..50: mol_type = mRNA
acgggggtggggcggggggggtatataaggcctgggatcccacgtccccg

SEQ ID NO: 18 - RNA, 50 nt, Human alphaherpesvirus 1
  source 1..50: mol_type = mRNA
atggccgctcccaaccgcgaccctccgggataccggtatgccgcggccat

(19) United States
(12) Patent Application Publication
(10) Pub. No.: US 2026/0014277 A1
(43) Pub. Date: Jan. 15, 2026

(54) NUCLEIC ACID, VECTORS, COMPOSITIONS, AND METHODS FOR ENHANCING EXPRESSION OF A PROTEIN OF INTEREST

(71) Applicant: CHILDREN'S HOSPITAL OF EASTERN ONTARIO RESEARCH INSTITUTE INC., Ottawa (CA)

(72) Inventors: TOMMY ALAIN, Ottawa (CA); HUY-DUNG HOANG, Ottawa (CA); TYSON GRABER, Ottawa (CA)

(21) Appl. No.: 18/993,869

(22) PCT Filed: Jul. 12, 2023
(86) PCT No.: PCT/CA2023/050935

§ 371 Date: Jan. 13, 2025

Related U.S. Application Data
(60) Provisional application No. 63/388,861, filed on Jul. 13, 2022.

(51) Int. Cl. A61K 48/00; A61K 38/19; A61P 35/00; C12N 15/86

(52) U.S. Cl. CPC A61K 48/005; A61K 38/193; A61P 35/00; C12N 15/86; C12N 2710/16643; C12N 2830/50

(57) ABSTRACT

The present invention is directed to nucleic acids, vectors, compositions, kits, and cell lines comprising US11 5′ leader sequence (SEQ ID NO: 1 or SEQ ID NO: 4). UL27 5′ leader sequence (SEQ ID NO: 7 or SEQ ID NO: 10), or UL19 5′ leader sequence (SEQ ID NO: 13 or SEQ ID NO: 16) whereby the leader sequences are capable of enhancing translation of a downstream gene encoding a protein of interest, resulting in increased protein production, expression or synthesis in HSV1 infected cells. The 5′ leader increases protein expression by several fold compared to protein expression in the absence of the leader sequence. Methods of increasing protein production, methods of increasing efficiency of existing gene therapies, and methods of treating a medical condition, cellular defect, disease or disorder are also provided.

### FIELD OF THE INVENTION

This invention relates to nucleic acid sequences; vectors, compositions, cells and kits comprising the nucleic acid sequences, which are capable of increasing translation of a gene encoding a protein of interest. More specifically the invention relates to nucleic acid sequences that increase protein production or expression when positioned upstream or 5′ to the gene encoding protein of interest and methods for increasing protein production are provided as well.

### BACKGROUND OF THE INVENTION

The use of viruses as vectors to deliver a therapeutic payload is becoming increasingly important in various biomedical applications such as viral vector-based vaccines, gene therapies, and oncolytic viruses (OVs). In the case of OVs, high intra-tumoural transgene expression is crucial to elicit optimal anti-tumour immune responses and therapeutic efficacy. Consequently, OV platforms in clinical and pre-clinical development are engineered to encode one or more of the following: 1) immunomodulatory host proteins or tumour associated antigens that can amplify anti-tumour immune responses, 2) suicide proteins intended to induce cancer cell death, and 3) reporter proteins that facilitate tracking and dosing (e.g., green fluorescent protein or luciferase).

The components that comprise a transgene expression cassette within a viral vector typically encode the transgene as an intron-less open reading frame (ORF) flanked by a upstream promoter and end with a poly(A) signal in the HSV1 genome. Remaining sequences from the multiple cloning sites (MCS) between the transcription start site (TSS) following the promoter and the start codon, or the stop codon and poly(A) signal, act as short 5′UTR and 3′UTR, respectively. Much effort was spent on ensuring high transgene transcription in pre-infected cells by incorporating a strong host promoter such as those from human phosphoglycerate kinase (PGK) or elongation factor 1a (EF1a), or from viruses such as the cytomegalovirus (CMV) immediate early promoter. However, any bottlenecks in the subsequent translation of the transgene mRNA into protein have not been thoroughly investigated under the assumption that differences in the rate of translation would be nominal if the transcript is highly expressed. The innate antiviral response to the presence of replicating viruses challenges this assumption; infected cells see dramatic changes in the subset of translated mRNAs, with replicating viruses actively reshaping the host protein synthesis machinery to favor production of viral proteins while impeding those of their host. Thus, translation rate of transgene mRNAs is likely to be altered in infected cells. Ultimately this could result in suboptimal transgene protein production and consequently compromise the therapeutic potential of the replicating viral vector by delivering viral units with inadequate specific activity to cancer patients.

Surprisingly, transgenes have not been optimized for their viral platform, and inadequate expression impacts therapeutic efficacy. An example is T-Vec; in a Phase I clinical trial, detected GM-CSF mRNA levels from fine needle aspirates of several different tumour types plateaued or fell below detection at a mid-range dose, suggesting that expression is sub-optimal in humans. In another Phase I trial, a clinical oncolytic vaccinia virus (VV) candidate also engineered to express GM-CSF, Jennerex-594 (JX594), failed to elicit detectable GM-CSF with low doses of intravenously injected virus (10⁵-106 viral plaque forming units; pfu per kg); in stark contrast, an order of magnitude increase in viral dose does yield a positive detection of this therapeutic protein. The requirement to increase dosing is however inopportune, as it is desirable to minimize viral dose in order to improve the safety profiles of these promising viral therapies. In other words, there is a want to increase the specific activity of an OV; non-OV based viral therapies, or any viral or non-viral gene delivery mechanisms to enhance targeting and expression of its transgene(s) while reducing the accompanying toxic effects of its vector or delivery vehicle.

### BRIEF DESCRIPTION OF DRAWINGS

FIGS. 1a, 1b, and 1c shows the representative nucleic acid sequences of the present invention.

FIG. 2 generally shows characterization of HSV1 individual transcript from RNA-seq coverage.

FIG. 2A shows total RNA-seq coverage of the HSV1 genome from 4T1 infected cells. Strand-specific RNA reads were mapped to HSV1 genome and separated by strand direction to avoid ambiguous mapping of overlapping genes.

FIG. 2B shows RNA-seq coverage of the US1 gene where intron-spanning reads were also detected and shown using Sashimi plot.

FIG. 2C shows RNA-seq coverage in the 5′ region of US1 gene. Lower plot show the region of the predicted TSS at nucleotide resolution.

FIG. 2D shows RNA-seq coverage at the 3′ region of US1 gene.

FIG. 2E provides a schematic overview of the workflow to identify HSV1 5′leaders from RNA-seq read, screen for translation enhancing 5′leaders specifically in HSV1-infected cells, and incorporate the 5′leader into transgene expression in an oncolytic HSV1 genome to test in a tumor model in vivo.

FIG. 3 generally shows HSV1 US11 5′ leader sequences enhance expression of protein reporters in HSV1-infected mammalian cells.

FIG. 3A is a schematic diagram of the mRNAs expressed from CAT reporter construct with/without HSV1 5′UTR.

FIG. 3B shows a translation reporter assay to screen for HSV1 5′ leader sequences that enhance translation during HSV1 infection. 4T1 cells infected with HSV-1716-GFP at an MOI of 5, then transfected with the CAT plasmid and a β-GAL expression plasmid that serve as a transfection control. Cells were lysed 24 hours post infection and CAT expression was quantified by ELISA, while β-GAL activity was quantified by colorimetric assay using ONPG substrate. Two-way ANOVA with Tukey's post-hoc test was performed. Only significant tests were shown. n=at least 3 biological replicates. Error bars indicate standard deviation (sd). * p<0.05, ** p<0.01.

FIG. 3C shows relative CAT mRNA expression from CAT translation reporter assay. 4T1 cells were treated as in (C), then lysed using Trizol. RT-qPCR was then used to quantified mRNA expression of CAT mRNA normalized to the expression of Rps20. Two-way ANOVA with Sidak's post-hoc test was performed. n=3 biological replicates. Only significant tests were shown. Error bars indicate standard deviation (sd). * p<0.05, ** p<0.01, **** p<0.0001.

FIG. 3D shows a schematic diagram of the pTK-Green plasmid that harbor the HSV1 5′ leader-reporter construct for insertion into the HSV1 TK gene and the resulting transcripts. The ribosome skipping sequence P2A is inserted between the luciferase CDS and GFP CDS, which allowed for synthesis of 2 proteins from one cistron.

FIG. 3E shows quantification of GFP fluorescence. Cells were transfected with LUC-GFP reporter plasmid, then infected with the HSV1 (JQ780693 for strain KOS) 4 hours post transfection at an MOI of 2.5. Images was taken at 24 hours post infection.

FIG. 3F shows Western Blot of lysate from 293T cells that were treated as in (B) with antibodies against GFP, anti-HSV1 or anti-β-Actin antibodies. (*): non-specific band.

FIG. 3G shows Quantification of GFP expression from the Western Blots in (F).

FIG. 3H shows RT-qPCR quantification of LUC-GFP mRNAs from the same experiment. Two-way ANOVA with Sidak's post-hoc test was performed. n=3 biological replicates. Error bars indicate standard deviation (sd). * p<0.05, ** p<0.01, **** p<0.0001, ns, non-significant.

FIG. 4 shows a recombinant HSV1 virus exhibits a US11 5′leader-dependent boost in GM-CSF expression.

FIG. 4A shows schematic illustration of the expression cassette insertion scheme from the pTK-CSF2-GFP plasmid into HSV1 genome (note that the TK gene is on the minus strand), and the resulting transcripts expressed from the inserted cassette.

FIG. 4B shows virus genotyping for confirmation of expression cassette insertion. PCR was done using HSV1 gDNA extracted from purified viruses to confirm the insertion of the leaderless CSF2-GFP cassette (˜400 bp) and US11 5′ leader-CSF2-GFP cassette (˜600 bp) into the TK region of HSV1 genome.

FIG. 4C shows fluorescence imaging of individual plaques of wild-type HSV1, HSV1 Csf2 and HSV1 US11-Csf2.

FIG. 4D shows quantification of GM-CSF production in culture supernatant of Vero cells infected with HSV1 expressing leaderless CSF2-GFP or US11 5′ leader-CSF2-GFP. Monolayer of Vero cells was infected with the indicated virus at an MOI of 5, and the culture supernatant was collected 24 hours post-infection. GM-CSF concentration was quantified by ELISA. One-way ANOVA with Dunnett's post-hoc test was performed. n=3 biological replicates. Error bars: +sd. **** p<0.0001.

FIG. 4E shows single-step growth curve of HSV1 Csf2 and HSV1 US11-Csf2. Monolayer of Vero cells was infected at an MOI of 5, then intracellular and extracellular virus was collected and titrated at the indicated time points.

FIG. 4F shows transcription level of HSV1 endogenous gene (US6) and transgenes. Monolayer of Vero cells was infected at an MOI of 5, then cells were lysed using Trizol at the indicated time points. mRNA abundance was quantified by RT-qPCR and normalized to Rps20.FIG. 5 generally shows the HSV1 US11 leader increases translation efficiency of a downstream transgene.

FIG. 5A shows fluorescence and phase contrast images of HSV1 infected Vero cells used for the polysome fractionation experiment in (B) and (C). Scale bar: 400 μm.

FIG. 5B shows polysome traces of Vero cells infected with HSV1 Csf2 or HSV1 US11-Csf2 at an MOI of 5. Cells were lysed at 24 hours post-infection for polysome fractionation.

FIG. 5C shows mRNA distribution in polysome fractions of Csf2 (upper panel) and the endogenous HSV1 transcripts US6 (middle panel) and US11 (lower panel), quantified by RT-qPCR. Two-sided t-test was performed. n=3 biological replicates. Error bars indicate standard deviation. **: p<0.01, *: p<0.05.

FIG. 5D shows mRNA distribution in non-translating fraction (subpolysome), poorly translating fraction (2-4 ribosomes) and highly translated fraction (>4 ribosomes) of Csf2 (upper panel), US6 (middle panel) and US11 (lower panel) transcripts. Multiple unpaired t-test was performed. n=3 biological replicates. Error bars indicate standard deviation (sd).***: p<0.001.

FIG. 5E shows quantification of GFP fluorescence of Vero cells transfected with pTK-CSF2-GFP plasmid with or without US11 leader sequence, co-transfected with poly(I: C) or infected immediately after with VSV or wild-type HSV1 at an MOI of 5.

FIG. 6 generally shows US11 5′leader enhances the antitumour effect of GM-CSF expressing HSV1.

FIG. 6A shows schematic overview of the in vivo study to characterize the effect of HSV1 US11-Csf2 on tumour inflammation and systemic antitumour T-cells. 10⁵ CT26 cells were injected in both flanks of BALB/c mice. When the tumour reached approximately 5×5 mm, two injections of 5×10⁵ PFU of the indicated virus were performed intratumourally two days apart (Day 0 and Day 2). Tumour size was measured every 2 days.

FIG. 6B shows intratumoural GM-CSF level of tumour treated with Leaderless or HSV1 US11-Csf2. Tumours as generated in (A) were excised one day after the second injection and homogenized in PBS. GM-CSF level was then quantified by ELISA. Two-tailed unpaired t-test was performed. n=3 biological replicates. Error bars: +sd.

FIG. 6C shows viral transcripts and inflammatory genes expression level in oHSV1 treated tumours. RNA from tumours in (B) were extracted with Trizol, then mRNA abundance of the indicated transcripts were quantified by RT-qPCR and normalized to Actb. Two-tailed unpaired t-test was performed. n=3 biological replicates. Error bars: +sd.

FIG. 6D shows systemic tumour-specific T-cell responses after oHSV1 injection, evaluated by IFNγ ELISPOT. Eight days after the first injection, splenocytes were isolated from mice, and co-cultured with/without UV-irradiated CT26 at a 2:1 responder-to-stimulator ratio for 24 hours. Representative ELISPOT wells are shown, as well as quantification of CT26-specific spots in the bar graph. Two-tailed unpaired t-test was performed. Error bars: +sd.

FIG. 6E shows the effect of Leaderless- or HSV1 US11-Csf2 treatment on tumour growth.

Number of mice is shown in brackets. Two-way ANOVA with Tukey's post-hoc test was performed. Error bars: +sd.

FIG. 6F shows Kaplan-Meier survival curve of mice treated with the Leaderless- or US11-Csf2 HSV1. Number of mice is shown in brackets.

FIG. 7 shows individual transcripts (RL2, UL15, US1, US12) identified by RNA-seq coverage on the positive strand (blue) and negative strand (red)

FIG. 8A-8D shows total RNA-seq coverage of the HSV1 genome. RNA-seq data from 4T1 infected with HSV1 previously published (Hoang et al., 2019). Strand-specific RNA reads were mapped to HSV1 (JQ780693.1) and separated by direction to avoid ambiguous mapping of overlapping genes. This graph shows the inset for every identified TSSs of all HSV1 gene.

FIG. 9 shows relative mRNA expression level of the 10 late genes selected as candidates (left heatmap), as well 4 immediate early genes (right heatmap). mRNA expression were obtained from a previously reported study by Rutkowski et al., 2015. Expression level was normalized as a percentage of the highest expression level across all time points.

FIG. 10 generally shows translation reporter screen for 5′UTRs that enhance translation during HSV1 infection.

FIG. 10A shows agarose gel visualization of other HSV1 5′leaders (including the rest of the 5′ leader in FIG. 3 amplified from total RNA of HSV1 infected cells. Negative controls (RNA from uninfected cells) confirmed the specificity of the PCRs products for HSV1 transcripts only. (*): unspecific band.

FIG. 10B shows translation reporter assay to screen for HSV-1 leader sequences that enhance translation during HSV-1 infection. 4T1 cells were transfected with the CAT plasmid and a β-GAL expression plasmid that serve as a transfection control. 8 hours post transfection, cells were infected with HSV-1716-GFP at an MOI of 5. Cells were lysed 18 hours post infection and CAT expression was quantified by ELISA, while β-GAL activity was quantified by colorimetric assay using ONPG substrate.

FIG. 10C shows predicted secondary structure and folding free energy of US11 (left panel) and UL27 (right panel) leaders using Vienna RNAfold33. Color scale bars represent base pairing probabilities.

FIG. 10D shows heatmap representing folding free energy of candidate HSV1 leaders, calculated using Vienna RNAfold.

FIG. 11 shows US11 leader enhancement is robust in different cell types and species. A monolayer of the African green monkey kidney cell line Vero, mouse breast cancer cell line 4T1, human pancreatic cancer cell line DU145 and human renal carcinoma cell lines 786-O was infected with HSV1 KOS leaderless or HSV1 US11 5′leader at an MOI of 0.1, then GFP fluorescence intensity was observed for 48 hours using the Incucyte live cell imaging system.

FIG. 12 shows leaderless transgene mRNA are poorly translated compared to viral mRNA. In the same polysome profiling experiment described in FIG. 4, the mRNA distribution of US6 and US11 were compared to that of leaderless Csf2 (FIG. 12A) or US11-Csf2 (FIG. 12B) mRNA.

FIG. 13 shows the size of individual tumours shown in FIG. 6E.

FIG. 14 shows raw data of individual wells from the IFNγ ELISPOT experiment in 6D.

FIG. 15 characterizes expression enhancement by US11 5′ leader in oncolytic HSV1.

FIG. 15A shows monolayer of Vero cells was infected with the indicated virus at a MOI of 5, and the culture supernatant was collected 24 hours post infection. GM-CSF concentration was quantified by ELISA. One-way ANOVA with Dunnett's post-hoc test was performed. n=3 biological replicates. Error bars: +sd. **** «p<0.0001.

FIG. 15B shows representative GFP fluorescence of HSV1 expressing Leaderless CSF2-GFP or US11 5′leader-CSF2-GFP. Monolayer of CT26 was infected with the indicated virus at an MOI of 5, then fluorescence microscopy images were taken at 24 hours post-infection.

FIG. 15C shows time course of GFP fluorescence of HSV1 expressing Leaderless CSF2-GFP or US11 5′leader-CSF2-GFP. Monolayer of CT26 was infected with the indicated virus at an MOI of 0.2, 1 or 5, then GFP fluorescence was monitored over 2 days post-infection using the Incucyte live cell imaging system.

FIG. 15D shows dose dependency analysis of secreted GM-CSF. CT26 cells were infected with the indicated virus at an MOI of 0.2, 1 or 5, then culture supernatant was collected 24 hours post-infection. GM-CSF concentration was quantified using ELISA. Two-way ANOVA with Sidak post-hoc test was performed. n=3 biological replicates. Error bars: +sd. **** p<0.0001.

### SUMMARY OF THE INVENTION

The invention relates to a nucleic acid comprising SEQ ID NO: 1 or a fragment comprising at least 180 nucleotides thereof, or a sequence that is at least 90% identical to SEQ ID NO: 1, where the nucleic acid does not comprise SEQ ID NO: 2, SEQ ID NO: 3, or both, and where, the nucleotide sequence does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 1.

In some embodiments, the invention relates to a nucleic acid comprising SEQ ID NO: 4 or a fragment comprising at least 180 nucleotides thereof, or a sequence that is at least 90% identical to SEQ ID NO: 4, where the nucleic acid does not comprise SEQ ID NO: 5, SEQ ID NO: 6, or both, and where, the nucleotide sequence does not comprise a fragment of SEQ ID NO: 5, 6 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 4.

In some embodiments, the invention relates to a nucleic acid comprising SEQ ID NO: 7 or a fragment comprising at least 180 nucleotides thereof, or a sequence that is at least 90% identical to SEQ ID NO: 7, where the nucleic acid does not comprise SEQ ID NO: 8, SEQ ID NO: 9, or both, and where, the nucleotide sequence does not comprise a fragment of SEQ ID NO: 8, 9 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 7.

In some embodiments, the invention relates to a nucleic acid comprising SEQ ID NO: 10 or a fragment comprising at least 180 nucleotides thereof, or a sequence that is at least 90% identical to SEQ ID NO: 16, where the nucleic acid does not comprise SEQ ID NO: 11, SEQ ID NO: 12, or both, and where, the nucleotide sequence does not comprise a fragment of SEQ ID NO: 11, 12 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 10.

In some embodiments, the invention relates to a nucleic acid comprising SEQ ID NO: 13 or a fragment comprising at least 180 nucleotides thereof, or a sequence that is at least 90% identical to SEQ ID NO: 13, where the nucleic acid does not comprise SEQ ID NO: 14, SEQ ID NO: 15, or both, and where, the nucleotide sequence does not comprise a fragment of SEQ ID NO: 14, 15 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 13.

In some embodiments, the invention relates to a nucleic acid comprising SEQ ID NO: 16 or a fragment comprising at least 180 nucleotides thereof, or a sequence that is at least 90% identical to SEQ ID NO: 16, where the nucleic acid does not comprise SEQ ID NO: 17, SEQ ID NO: 18, or both, and where, the nucleotide sequence does not comprise a fragment of SEQ ID NO: 17, 18 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 16.

In an embodiment, there is provided a nucleic acid comprising SEQ ID NO: 1 or a fragment comprising at least 180 nucleotides, or a sequence that is at least 90% identical to SEQ ID NO: 1, where the nucleic acid does not comprise SEQ ID NO: 2, SEQ ID NO: 3, or both. In an alternate embodiment, there is provided a sequence that is at least 90% identical to SEQ ID NO: 1, or a fragment comprising at least 180 nucleotides where the nucleic acid does not comprise SEQ ID NO: 2, SEQ ID NO: 3, or both. The nucleic acid described above does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 1.

In an alternate embodiment, there is provided a nucleic acid comprising SEQ ID NO: 4 i.e. the RNA counterpart of SEQ ID NO: 1, or a fragment comprising at least 180 nucleotides, or a sequence that is at least 90% identical to SEQ ID NO: 4, where the nucleic acid does not comprise SEQ ID NO: 5 (i.e. RNA counterpart of SEQ ID NO: 2), SEQ ID NO: 6 (i.e. RNA counterpart of SEQ ID NO: 3), or both. In an alternate embodiment, there is provided a sequence that is at least 90% identical to SEQ ID NO: 4, or a fragment comprising at least 180 nucleotides where the nucleic acid does not comprise SEQ ID NO: 5, SEQ ID NO: 6, or both. The nucleic acid described above does not comprise a fragment of SEQ ID NO: 5, 6 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 4.

In an alternate embodiment, there is provided a nucleic acid comprising SEQ ID NO: 10 i.e. the RNA counterpart of SEQ ID NO: 7, or a fragment comprising at least 180 nucleotides, or a sequence that is at least 90% identical to SEQ ID NO: 10, where the nucleic acid does not comprise SEQ ID NO: 11 (i.e. RNA counterpart of SEQ ID NO: 8), SEQ ID NO: 12 (i.e. RNA counterpart of SEQ ID NO: 9), or both. In an alternate embodiment, there is provided a sequence that is at least 90% identical to SEQ ID NO: 10, or a fragment comprising at least 180 nucleotides where the nucleic acid does not comprise SEQ ID NO: 11, SEQ ID NO: 12, or both. The nucleic acid described above does not comprise a fragment of SEQ ID NO: 11, 12 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 10.

The nucleotide sequence or the nucleic acid recited in this invention does not comprise at least 250, 500, 1000 or more continuous nucleotides of Human herpesvirus 1 strain KOS, complete genome defined by NCBI Accession number: JQ673480.1 GI: 380776962 or by Accession number JQ780693.1 GI: 384597744, or a sequence which is 95% identical thereto.

In an embodiment of the invention, there is provided a vector comprising the SEQ ID NO: 1, SEQ ID NO: 7, or SEQ ID NO: 13 or a fragment comprising at least 180 nucleotides, or a sequence that is at least 90% identical to SEQ ID NO: 1, SEQ ID NO: 7, or SEQ ID NO: 13, or; a sequence that is at least 90% identical to SEQ ID NO: 1, SEQ ID NO: 7, or SEQ ID NO: 13 or a fragment comprising at least 180 nucleotides and a promoter, a nucleotide sequence encoding a protein of interest, one or more regulatory sequences, one or more restriction endonuclease or cloning sites, and one or more polyadenylation sites or any combination thereof. An alternate embodiment comprising the RNA counterpart of SEQ ID NOs: 1, 7 and 13 (i.e. SEQ ID NOs: 4, 10 and 16) is also provided.

In some alternate embodiments, the nucleic acid may be inserted in a gene delivery vehicle, some non-limiting examples of which are a plasmid, an expression cassette, a live virus, a DNA or RNA construct or recombinant nucleotide construct, an intron-less open reading frame, a nanoparticle, or a lipid nanoparticle. In some embodiments, the gene delivery vehicles recited above may be HSV or HSV1 based.

In an embodiment of the invention, there is also provided a cell, a composition or a kit comprising the nucleic acid or the vector recited above. Specifically, the nucleic acid comprises SEQ ID NO: 1, SEQ ID NO: 7, SEQ ID NO: 13 or their RNA counterparts i.e. SEQ ID NO: 4, SEQ ID NO: 10, or SEQ ID NO: 16. In some embodiments, both nucleic acid and vector could be present in combination.

In the embodiments recited above, when the nucleic acid comprises SEQ ID NO: 1, the nucleic acid does not comprise SEQ ID NO: 2, SEQ ID NO: 3, or both; when the nucleic acid comprises SEQ ID NO: 4, the nucleic acid does not comprise SEQ ID NO: 5, SEQ ID NO: 6, or both; when the nucleic acid comprises SEQ ID NO: 7, the nucleic acid does not comprise SEQ ID NO: 8, SEQ ID NO: 9 or both; when the nucleic acid comprises SEQ ID NO: 10, the nucleic acid does not comprise SEQ ID NO: 11, SEQ ID NO: 12, or both; when the nucleic acid comprises SEQ ID NO: 13, the nucleic acid does not comprise SEQ ID NO: 14, SEQ ID NO: 15, or both; when the nucleic acid comprises SEQ ID NO: 16, the nucleic acid does not comprise SEQ ID NO: 17, SEQ ID NO: 18, or both.

In case of SEQ ID NO: 1, the nucleic acid described above may not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 1.

In case of SEQ ID NO: 4, the nucleic acid described above may not comprise a fragment of SEQ ID NO: 5, 6 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 4.

In case of SEQ ID NO: 7, the nucleic acid described above may not comprise a fragment of SEQ ID NO: 8, 9 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 7.

In case of SEQ ID NO: 10, the nucleic acid described above may not comprise a fragment of SEQ ID NO: 11, 12 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 10.

In case of SEQ ID NO: 13, the nucleic acid described above may not comprise a fragment of SEQ ID NO: 14, 15 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 13.

In case of SEQ ID NO: 16, the nucleic acid described above may not comprise a fragment of SEQ ID NO: 17, 18 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 16.

In an embodiment of the invention, there is provided a method of producing a protein of interest in a cell comprising, wherein the method comprises administering a nucleic acid to the cell. The nucleic acid being administered comprises a) a promoter, b) SEQ ID NO: 1, SEQ ID NO: 7 or SEQ ID NO: 13; and c) a sequence encoding a protein of interest which is expressed from the nucleic acid in the cell. An alternate embodiment, where the method employs the RNA counterpart of SEQ ID NO: 1 (i.e. SEQ ID NO: 4), SEQ ID NO: 7 (i.e. SEQ ID NO: 10), or SEQ ID NO: 13 (i.e. SEQ ID NO: 16) is also provided.

In an alternate embodiment, there is provided a method of increasing expression, synthesis, or production of a protein of interest in a cell comprising the steps of: administering a nucleic acid to the cell, where the nucleic acid comprises a) a promoter; b) SEQ ID NO: 1, SEQ ID NO: 7 or SEQ ID NO: 13; and c) a sequence encoding a protein of interest which is expressed from the nucleic acid in the cell, where the increase in expression, synthesis or production of a protein of interest is relative to a similar step of administering a nucleic acid in the absence of SEQ ID NO:1, SEQ ID NO: 7, or SEQ ID NO: 13. An alternate embodiment, where the method employs the RNA counterpart of SEQ ID NO: 1 (i.e. SEQ ID NO: 4), SEQ ID NO: 7 (i.e. SEQ ID NO: 10), or SEQ ID NO: 13 (i.e. SEQ ID NO: 16) is also provided.

In an embodiment of the invention there is provided a method of improving or treating a medical condition, cellular defect or disease in a subject. The method comprises administering a nucleic acid to the cells of the subject showing the medical condition, cellular defect or disease, where the nucleic acid comprises a) a promoter, b) SEQ ID NO: 1, SEQ ID NO: 7 or SEQ ID NO: 13; and c) a sequence encoding a protein of interest which is expressed from the nucleic acid in the cells. The protein of interest selected is capable of improving or treating the medical condition, cellular defect or disease in the subject, and therefore, the expression, synthesis, or production of the protein of interest in the cells of the subject improves or treats the medical condition, cellular defect or disease in the subject. An alternate method, where the nucleic acid comprises SEQ ID NO: 4, SEQ ID NO: 7 (i.e. SEQ ID NO: 10), or SEQ ID NO: 13 (i.e. SEQ ID NO: 16) is also provided.

### DETAILED DESCRIPTION

The following description is of preferred embodiments by way of example only and without limitation to the combination of features necessary for carrying the invention into effect.

All terms are intended to be understood as they would be understood by a person skilled in the art. Unless defined otherwise, all technical and scientific terms used herein have the same meaning as commonly understood by one of ordinary skill in the art to which the disclosure pertains. The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application. Accordingly, the terminology used herein is for the purpose of describing particular embodiments only, and is not intended to be limiting

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof and their grammatical equivalents as used herein, can be used interchangeably. These terms can convey that any and all combinations are specifically contemplated. The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” “alternate embodiment”, or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value. The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “isolated” and its grammatical equivalents as used herein refer to the removal of a nucleic acid from its natural environment. Whether the nucleic acid is removed from nature (including genomic DNA and mRNA) or synthesized (including cDNA) and/or amplified under laboratory conditions, it is to be understood, however, that nucleic acids and proteins can be formulated with diluents or adjuvants and still for practical purposes be isolated. For example, nucleic acids typically are mixed with an acceptable carrier or diluent when used for introduction into cells.

As used herein, the term “gene” refers to a nucleic acid molecule capable of being used to produce mRNA, antisense RNA, siRNA, shRNA, miRNA, and the like. Genes may or may not be capable of being used to produce a functional protein. Genes can include both coding and non-coding regions (e.g., introns, regulatory elements including promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule. Reference to a “gene” also includes within its scope reference to genes having a contiguous sequence, thus defining contiguous nucleic acid entities, as defined herein, or a non-contiguous sequence thus defining a non-contiguous nucleic acid entity as defined herein. In certain embodiments, the term “gene” includes within its scope the open reading frame encoding specific polypeptides, introns, and adjacent 5′ and 3′ non-coding nucleotide sequences involved in the regulation of expression. In this regard, the gene may further comprise control sequences such as promoters, enhancers, termination and/or polyadenylation signals that are naturally associated with a given gene, or heterologous control sequences. The gene sequences may be cDNA or genomic DNA or a fragment thereof. The gene may be introduced into an appropriate vector for extrachromosomal maintenance or for introduction into a host.

“Genome” as used herein refers to the entirety of an organism's hereditary information, represented by genes and non-coding sequences of DNA, either chromosomal or non-chromosomal genetic elements such as, linear polynucleotides, e.g., including the gene(s) to be assembled and/or recombined. Thus, the term “genome” is intended to include the entire DNA of an organism, including the nuclear DNA component, chromosomal or extrachromosomal DNA, as well as the cytoplasmic domain (e.g., mitochondrial DNA).

The terms “Nucleic Acid(s)”, “Polynucleotide(s)”, “oligonucleotide(s)”, or “nucleotide(s)”, or any grammatical equivalent as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. Homology is generally inferred from sequence identity between two or more nucleic acids (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window.

Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligences, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. Nucleic acids can also be described with reference to a starting nucleic acid, e.g., they can be 50%, 60%, 70%, 75%, 80%, 85%, 90%, 98%, 99% or 100%>identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid sequences mean that a nucleic acid or amino acid sequence comprises a sequence that has at least 90% sequence identity or more, at least 95%, at least 98% and at least 99%), compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 25 bases in length, 50 bases in length, 100 bases in length, 125 bases in length, 150 bases in length and in embodiments, the sequences are substantially identical over at least about 180 bases. In embodiments, the sequences are substantially identical over the entire length of the coding regions.

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an episomal expression vector, which is able to replicate in a host cell, and persists as an extrachromosomal segment of DNA within the host cell in the presence of appropriate selective pressure. Vector also can comprise a selectable marker gene. The term “selectable marker gene” as used herein refers to a nucleic acid sequence that allows cells expressing the nucleic acid sequence to be specifically selected for or against, in the presence of a corresponding selective agent.

The term “coding sequence” or “sequence encoding” as used herein refers to a segment of a polynucleotide that codes for protein. The region or sequence is bounded nearer the 5′end by a start codon and nearer the 3′ end with a stop codon. Coding sequences can also be referred to as open reading frames. The present invention is further directed to a nucleotide construct comprising the nucleic acid as described above operatively linked to one or more regulatory elements or regulatory regions. By “regulatory element” or “regulatory region”, it is meant a portion of nucleic acid typically, but not always, upstream of a gene, and may be comprised of either DNA or RNA, or both DNA and RNA. Regulatory elements may include those which are capable of mediating organ specificity, or controlling developmental or temporal gene activation. Furthermore, “regulatory element” includes promoter elements, core promoter elements, elements that are inducible in response to an external stimulus, elements that are activated constitutively, or elements that decrease or increase promoter activity such as negative regulatory elements or transcriptional enhancers, respectively. By a nucleotide sequence exhibiting regulatory element activity it is meant that the nucleotide sequence when operatively linked with a coding sequence of interest functions as a promoter, a core promoter, a constitutive regulatory element, a negative element or silencer (i.e. elements that decrease promoter activity), or a transcriptional or translational enhancer.

The present invention further includes vectors comprising the nucleic acids as described above. Suitable expression vectors for use with the nucleic acid sequences of the present invention include, but are not limited to, plasmids, phagemids, viral particles and vectors, phage and the like. For insect cells, baculovirus expression vectors are suitable. The entire expression vector, or a part thereof, can be integrated into the host cell genome.

Those skilled in the art will understand that a wide variety of expression systems can be used to produce the proteins or fragments thereof as defined herein. Different host cells have characteristic and specific mechanisms for the post-translational processing and modification of proteins and gene products. Appropriate cell lines or host systems can be chosen by one skilled in the art to ensure the correct modification and processing of the expressed cardiac stem cell proliferation protein.

The term “operably linked” as used herein refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The term “induce”, “induction” and its grammatical equivalents as used herein refer to an increase in nucleic acid sequence transcription, promoter activity and/or expression brought about by a transcriptional regulator, relative to some basal level of transcription or control systems used. Increase in nucleic acid sequence transcription, promoter activity and/or expression can also be brought about by a translation regulator such as a translation enhancing UTR sequence.

Regulatory elements as used herein, also includes elements that are active following transcription initiation or transcription, for example, regulatory elements that modulate gene expression such as translational and transcriptional enhancers, translational and transcriptional repressors, and mRNA stability or instability determinants. In the context of this disclosure, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns, or 3′ of the coding region sequence may also contribute to the regulation of expression of a coding region of interest. A regulatory element may also include those elements located downstream (3′) to the site of transcription initiation, or within transcribed regions, or both. In the context of the present invention a post-transcriptional regulatory element may include elements that are active following transcription initiation, for example translational and transcriptional enhancers, translational and transcriptional repressors, and mRNA stability determinants.

The regulatory elements, or fragments thereof, may be operatively associated (operatively linked) with heterologous regulatory elements or promoters in order to modulate the activity of the heterologous regulatory element. Such modulation includes enhancing or repressing transcriptional activity of the heterologous regulatory element, modulating post-transcriptional events, or both enhancing/repressing transcriptional activity of the heterologous regulatory element and modulating post-transcriptional events. For example, one or more regulatory elements, or fragments thereof, may be operatively associated with constitutive, inducible, tissue specific promoters or fragment thereof, or fragments of regulatory elements, for example, but not limited to TATA or GC sequences may be operatively associated with the regulatory elements of the present invention, to modulate the activity of such promoters within plant, insect, fungi, bacterial, yeast, or animal cells.

There are several types of regulatory elements, including those that are developmentally regulated, inducible and constitutive. A regulatory element that is developmentally regulated, or controls the differential expression of a gene under its control, is activated within certain organs or tissues of an organ at specific times during the development of that organ or tissue. However, some regulatory elements that are developmentally regulated may preferentially be active within certain organs or tissues at specific developmental stages, they may also be active in a developmentally regulated manner, or at a basal level in other organs or tissues within a plant as well.

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter. Some non-limiting examples are CMV, EF1a, CAG, PGK, TRE, U6, and UAS.

By “promoter” it is meant the nucleotide sequences at the 5′ end of a coding region, or fragment thereof that contain all the signals essential for the initiation of transcription and for the regulation of the rate of transcription. There are generally two types of promoters, inducible and constitutive promoters.

A constitutive promoter directs the expression of a gene throughout the various parts of an organism and/or continuously throughout development of an organism. Any suitable constitutive promoter may be used to drive the expression of the proteins or fragments thereof as described herein.

The term “constitutive” as used herein does not necessarily indicate that a gene is expressed at the same level in all cell types, but that the gene is expressed in a wide range of cell types, although some variation in abundance is often observed.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off with an inducer at certain stages of development of an organism or in a particular tissue. An inducible promoter is a promoter that is capable of directly or indirectly activating transcription of one or more DNA sequences or genes in response to an inducer. In the absence of an inducer the DNA sequences or genes will not be transcribed. Typically the protein factor that binds specifically to an inducible promoter to activate transcription is present in an inactive form which is then directly or indirectly converted to the active form by the inducer. The inducer can be a chemical agent such as a protein, metabolite, growth regulator, or a physiological stress imposed directly by heat, cold, or toxic elements or indirectly through the action of a pathogen or disease agent such as a virus. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters, isopropyl-β-thiogalactopyranoside (IPTG) inducible promoter.

The nucleic acid and other constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3 prime end of the mRNA precursor. E.g. SV-40 polyadenylation site.

The gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

The term “transcriptional regulator” or “cis-acting regulatory elements” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” or “translation enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

“Patient” or “subject” as used herein refers to a mammalian subject diagnosed with or suspected of having or developing a medical condition, cellular defect or a disease or disorder, e.g. a proliferative disorder such as cancer. In some embodiments, the term “patient” refers to a mammalian subject with a higher than average likelihood of developing a proliferative disorder such as cancer.

“Patient in need thereof or” subject in need thereof is referred to herein as a patient diagnosed with or suspected of having a cellular defect, medical condition, disease or disorder, for instance, but not restricted to a proliferative disorder such as cancer. In some cases, a cancer is a solid tumour or a hematologic malignancy. In some instances, the cancer is a solid tumour. In other instances, the cancer is a hematologic malignancy. In some cases, the cancer is a metastatic cancer. In some cases, the cancer is a relapsed or refractory cancer. In some instances, the cancer is a solid tumour. Exemplary solid tumours include, but are not limited to, anal cancer; appendix cancer; bile duct cancer (i.e., cholangiocarcinoma); bladder cancer; brain tumour; breast cancer; cervical cancer; colon cancer; cancer of Unknown Primary (CUP); esophageal cancer; eye cancer; fallopian tube cancer;
gastroenterological cancer; kidney cancer; liver cancer; lung cancer; medulloblastoma; melanoma; oral cancer; ovarian cancer; pancreatic cancer; parathyroid disease; penile cancer; pituitary tumour; prostate cancer; rectal cancer; skin cancer; stomach cancer; testicular cancer; throat cancer; thyroid cancer; uterine cancer; vaginal cancer; or vulvar cancer. In some embodiments leukemia can be, for instance, acute lymphoblastic leukemia (ALL), acute myeloid leukemia (AML), chronic lymphocytic leukemia (CLL) and chronic myeloid leukemia (CML).

“Administering” is referred to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise a target cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment”, “treating”, “amelioration” or its grammatical equivalents refers to obtaining a desired pharmacologic and/or physiologic effect. In embodiments, the effect is therapeutic, i.e., the effect partially or completely cures a disease and/or adverse symptom attributable to the disease.

### Embodiments

Without wishing to be bound by theory or experimental results, the following paragraphs describe the nature of the invention by way of examples only. The experiments, or specific examples of materials, promoters, enhancers, regulatory elements, vectors, cell lines and constructs described should not be construed as limiting the scope of the invention. A person skilled in the art would readily understand and appreciate that other materials, promoters, enhancers, regulatory elements, vectors, cell lines and constructs not specifically described also form part of the invention.

The present invention relates to nucleic acid sequences; vectors, compositions, kits, and cells comprising the nucleic acid sequences and methods for increasing translation of a protein of interest.

Transgenes have been used to deliver therapeutic payloads for improving the efficacy of gene therapy, oncolytic virus immunotherapy, and viral vector vaccine platforms. Transgenes encoded within replication-competent virotherapies are designed to be highly transcribed, but protein synthesis is often negatively affected by viral infection, compromising the amount of therapeutic protein delivered and the efficacy of the corresponding therapies. Standard transgene mRNAs are sub-optimally translated in the infected cells. The current invention presents a method to identify viral translation enhancing 5′ leaders that boost mRNA translation.

Recent studies have revealed that various translation enhancer motifs found in the leader sequences of viral mRNAs, including non-templated poly(A) leaders in poxviruses or ICP27-interacting motifs in HSV1 mRNA leader are capable of facilitating nuclear export. Viral internal ribosome entry sites (IRES) such as those based on encephalomyocarditis virus (EMCV) are also used in recombinant gene expression to enhance translation of downstream genes or open reading frames (ORFs). However, the inclusion of translation enhancers has not been reported for clinically relevant Oncolytic Viruses (OVs), including oncolytic poxviruses and HSV1. For example, T-VEC GM-CSF expression cassette does not contain a functionally authentic viral 5′leader, but residual cloning sequences of approximately 100 bp originating from the MCS of the plasmid pcDNA3 are used in the cloning process.

Using RNA-Seq reads, the transcription start sites and 5′ leaders of Herpes Simplex Virus-1 (HSV1) genes from pre-infected cells were determined. HSV1 5′leaders that mediate high translation efficiency of downstream cistron mRNAs and display superior activity during viral replication were uncovered. The 5′leaders were inserted into GM-CSF expression cassette in oncolytic HSV1 and the translationally adapted oncolytic virus was compared with the conventional, leaderless virus in vitro and in mouse model. Oncolytic viruses are armed with transgenes that code for critical therapeutic payloads, however, the inventors determined that adding a viral 5′leader to the transgene mRNA enhances translation and boosts payload expression in infected cells, ultimately resulting in improved viral-mediated anti-tumour efficacy.

Screening the identified leaders for translation activity using a heterologous reporter identified the 5′ leader of the late viral gene US11. It was therefore hypothesized that synthesis of therapeutic payloads from replicating oncolytic platforms may be enhanced by incorporation of a viral nucleic acid sequence, potentially a 5′ leader or UTR sequence. RNA-Seq data of HSV1 viral genome was therefore carried out to identify sequences that were capable of enhancing protein expression. HSV-infected cancer cells were isolated and RNA-seq data was obtained to identify HSV1 sequences or 5′ leaders that mediate high translation efficiency of downstream cistron during viral replication. It was observed that US11 5′ leader sequence (i.e. SEQ of SEQ ID NO: 1) from HSV1 viral genome is capable of enhancing protein expression in cells by several fold when positioned upstream or 5′ to downstream cistron or gene encoding a protein of interest in HSV1 infected cells. An RNA counterpart of the US11 5′ leader sequence (i.e. sequence of SEQ ID NO: 4) is also capable of showing similar results. It was observed that inclusion of a 5′ leader in expression cassette incorporated into an HSV1 genome boosted translation of a protein of interest (e.g. GM-CSF) in vitro and in vivo. Importantly, treatment with this translation-enhanced oncolytic HSV1 showed superior anti-tumour immune activity and improved survival in a syngeneic mouse model of colorectal cancer as compared to leaderless-GM-CSF HSV1. This demonstrates the therapeutic value of identifying and incorporating vector-specific cis-acting sequences that confer increased protein synthesis on transgene expression.

Accordingly, the present invention is directed to nucleic acids, vectors, compositions, kits, and cell lines comprising SEQ ID NO: 1 or SEQ ID NO: 4, where the nucleic acid additionally comprises a gene encoding a protein of interest, and optionally comprises promoters, translation enhancers or other regulatory elements. More specifically, transcription of SEQ ID NO: 1 or 4 causes increased translation of the gene encoding the protein of interest, resulting in enhanced protein expression in HSV or HSV1 infected cells.

### US11 5′ Leader

With the use of RNA sequence mapping, the US11 5′ leader nucleotide sequence was identified and is recited below:

5′GGCCAGAACCGCCGTGCACGACCCGGAGCGTCCCCTGCTGCGCTCTC
CCGGGCTGCTGCCCGAAATCGCCCCCAACGCATCCTTGGGTGTGGCACA
TCGAAGAACCGGCGGGACCGTGACCGACAGTCCCCGTAATCCGGTAACC
CGTTGAGTCCCGGGTACGACCATCACCCGAGTCTCTGGGCGGAGGGTGG
TTCCCCCCCGTGTCTCTCGAG3′ (hereinafter SEQ ID NO:
1)

The sequence of SEQ ID NO: 1 was cross-referenced against HSV1 KOS strain using NCBI's BLAST database and the sequence found was 100% identical with SEQ ID NO: 1.

US11 5′Leader sequence:
>gi|384597744|gb|JQ780693.1|: c144480-144266 Human
herpesvirus 1 strain KOS, complete genome
5′GGCCAGAACCGCCGTGCACGACCCGGAGCGTCCCCTGCTGCGCTCTC
CCGGGCTGCTGCCCGAAATCGCCCCCAACGCATCCTTGGGTGTGGCACA
TCGAAGAACCGGCGGGACCGTGACCGACAGTCCCCGTAATCCGGTAACC
CGTTGAGTCCCGGGTACGACCATCACCCGAGTCTCTGGGCGGAGGGTGG
TTCCCCCCCGTGTCTCTCGAG3′

It was also observed that US11 5′ leader sequence is one of the highly conserved sequences of HSV1 genome across various identified strains. The NCBI BLASTN database also showed that the sequences upstream and downstream of US11 5′ leader are highly conserved as well.

5′GCCGACGTACGCGATGAGATCAATAAAAGGGGGCGTGAGGACCGGGA
GGC3′ (sequence upstream of SEQ ID NO: 1 in HSV1
viral genome, hereinafter SEQ ID NO: 2)
5′ATGAGCCAGACCCAACCCCCGGCCCCAGTTGGGCCGGGCGACCCAGA
TGT3′ (sequence downstream of SEQ ID NO: 1 in
HSV1 viral genome, hereinafter SEQ ID NO: 3)

As noted earlier, the desired effect of increased protein expression is achievable using the RNA counterpart of SEQ ID NO: 1 recited above. The RNA counterpart of the US11 5′ leader sequence, upstream and downstream sequences are provided below.

5′GGCCAGAACCGCCGUGCACGACCCGGAGCGUCCCCUGCUGCGCUCUC
CCGGGCUGCUGCCCGAAAUCGCCCCCAACGCAUCCUUGGGUGUGGCACA
UCGAAGAACCGGCGGGACCGUGACCGACAGUCCCCGUAAUCCGGUAACC
CGUUGAGUCCCGGGUACGACCAUCACCCGAGUCUCUGGGCGGAGGGUGG
UUCCCCCCCGUGUCUCUCGAG3′ (the RNA counterpart of
SEQ ID NO: 1, hereinafter SEQ ID NO: 4)
5′GCCGACGUACGCGAUGAGAUCAAUAAAAGGGGGCGUGAGGACCGGGA
GGC3′ (the RNA counterpart of SEQ ID NO: 2 i.e.
sequence upstream of SEQ ID NO: 4, hereinafter
SEQ ID NO: 5)
5′AUGAGCCAGACCCAACCCCCGGCCCCAGUUGGGCCGGGCGACCCAGA
UGU3′ (the RNA counterpart of SEQ ID NO: 3 i.e.
sequence downstream of SEQ ID NO: 4, hereinafter
SEQ ID NO: 6)

### UL27 5′ Leader

With the use of RNA sequence mapping, the UL27 5′ leader nucleotide sequence was identified and is recited below:

5′ACACTCTTTGCCTCGGTCTACCGGTGCGGGGAGCTCGAGTTGCGCCG
CCCGGACTGCAGCCGCCCGACCTCCGAAGGTCGTTACCGTTACCCGCCC
GGCGTATATCTCACGTACGACTCCGACTGTCCGCTGGTGGCCATCGTCG
AGAGCGCCCCCGACGGCTGTATCGGCCCCCGGTCGGTCGTGGTCTACGA
CCGAGACGTTTTCTCGATCCTCTACTCGGTCCTCCAGCACCTCGCCCCC
AGGCTACCTGACGGGGGGCACGACGGGCCCCCGTAGTCCCGCC3′
(hereinafter SEQ ID NO: 7)

The sequence of SEQ ID NO: 7 was cross-referenced against HSV1 KOS strain using NCBI's BLAST database and the sequence found was 100% identical with SEQ ID NO: 7.

UL27 5′Leader sequence:
>gi|384597744|gb|JQ780693.1|: c55744-55459 Human
herpesvirus 1 strain KOS, complete genome
5′ACACTCTTTGCCTCGGTCTACCGGTGCGGGGAGCTCGAGTTGCGCCG
CCCGGACTGCAGCCGCCCGACCTCCGAAGGTCGTTACCGTTACCCGCCC
GGCGTATATCTCACGTACGACTCCGACTGTCCGCTGGTGGCCATCGTCG
AGAGCGCCCCCGACGGCTGTATCGGCCCCCGGTCGGTCGTGGTCTACGA
CCGAGACGTTTTCTCGATCCTCTACTCGGTCCTCCAGCACCTCGCCCCC
AGGCTACCTGACGGGGGGCACGACGGGCCCCCGTAGTCCCGCC3′

It was also observed that UL27 5′ leader sequence is one of the highly conserved sequences of HSV1 genome across various identified strains. The NCBI BLASTN database also showed that the sequences upstream and downstream of UL27 5′ leader are highly conserved as well.

5′CCACTCAGCGCGCCGCCTGGCGATATATTCGCGAGCTGATTATCGCC
ACC3′ (sequence upstream of SEQ ID NO: 7 in HSV1
viral genome, hereinafter SEQ ID NO: 8)
5′ATGCACCAGGGCGCCCCCTCGTGGGGGCGCCGGTGGTTCGTCGTATG
GGC3′ (sequence downstream of SEQ ID NO: 7 in
HSV1 viral genome, hereinafter SEQ ID NO: 9)

As noted earlier, the desired effect of increased protein expression is achievable using the RNA counterpart of SEQ ID NO: 7 recited above. The RNA counterpart of the UL27 5′ leader sequence, upstream and downstream sequences are provided below.

5′ACACUCUUUGCCUCGGUCUACCGGUGCGGGGAGCUCGAGUUGCGCCG
CCCGGACUGCAGCCGCCCGACCUCCGAAGGUCGUUACCGUUACCCGCCC
GGCGUAUAUCUCACGUACGACUCCGACUGUCCGCUGGUGGCCAUCGUCG
AGAGCGCCCCCGACGGCUGUAUCGGCCCCCGGUCGGUCGUGGUCUACGA
CCGCGACGUUUUCUCGAUCCUCUACUCGGUCCUCCAGCACCUCGCCCCC
AGGCUACCUGACGGGGGGCACGACGGGCCCCCGUAGUCCCGCC3′
(the RNA counterpart of SEQ ID NO: 7, hereinafter
SEQ ID NO: 10)
5′CCACUCAGCGCGCCGCCUGGCGAUAUAUUCGCGAGCUGAUUAUCGCC
ACC3′ (the RNA counterpart of SEQ ID NO: 8 i.e.
sequence upstream of SEQ ID NO: 10, hereinafter
SEQ ID NO: 11)
5′AUGCACCAGGGCGCCCCCUCGUGGGGGCGCCGGUGGUUCGUCGUAUG
GGC3′ (the RNA counterpart of SEQ ID NO: 9 i.e.
sequence downstream of SEQ ID NO: 10, hereinafter
SEQ ID NO: 12).

UL19 5′ Leader

With the use of RNA sequence mapping, the UL19 5′ leader nucleotide sequence was identified and is recited below:

5′GGTCTGTTGGGGACACTGGGTTCTCCTGGAACGAGGCCGCAGCCTTC
TCCCGGTGCCTTTCCCCCCCGACCGACACCCGGCCTCTCACACAGCATC
CCCCGCCTTTTTGGGTCCGGGCCCGTCGTGTCTTTCGGTGGACCTTGGG
CCGTCGGGCACGTACACGGGTGGCCGGGCGTTGGGGTGGATCTTAGCCT
CCCCGGGCCAATATCGCTAGAGACAGCCGATCTCCACGCGACCCC3′
(hereinafter SEQ ID NO: 13)

The sequence of SEQ ID NO: 13 was cross-referenced against HSV1 KOS strain using NCBI's BLAST database and the sequence found was 100% identical with SEQ ID NO: 13.

UL19 5′ leader sequence:
>gi|384597744|gb|JQ780693.1|: c40421-40183 Human
herpesvirus 1 strain KOS, complete genome
5′GGTCTGTTGGGGACACTGGGTTCTCCTGGAACGAGGCCGCAGCCTTC
TCCCGGTGCCTTTCCCCCCCGACCGACACCCGGCCTCTCACACAGCATC
CCCCGCCTTTTTGGGTCCGGGCCCGTCGTGTCTTTCGGTGGACCTTGGG
CCGTCGGGCACGTACACGGGTGGCCGGGCGTTGGGGTGGATCTTAGCCT
CCCCGGGCCAATATCGCTAGAGACAGCCGATCTCCACGCGACCCC3′

It was also observed that UL19 5′ leader sequence is one of the highly conserved sequences of HSV1 genome across various identified strains. The NCBI BLASTN database also showed that the sequences upstream and downstream of UL19 5′ leader are highly conserved as well.

5′ACGGGGGTGGGGCGGGGGGGGTATATAAGGCCTGGGATCCCACGTCC
CCG3′ (sequence upstream of SEQ ID NO: 13 in HSV1
viral genome, hereinafter SEQ ID NO: 14)
5′ATGGCCGCTCCCAACCGCGACCCTCCGGGATACCGGTATGCCGCGGC
CAT3′ (sequence downstream of SEQ ID NO: 13 in
HSV1 viral genome, hereinafter SEQ ID NO: 15)

As noted earlier, the desired effect of increased protein expression is achievable using the RNA counterpart of SEQ ID NO: 13 recited above. The RNA counterpart of the UL19 5′ leader sequence, upstream and downstream sequences are provided below.

5′GGUCUGUUGGGGACACUGGGUUCUCCUGGAACGAGGCCGCAGCCUUC
UCCCGGUGCCUUUCCCCCCCGACCGACACCCGGCCUCUCACACAGCAUC
CCCCGCCUUUUUGGGUCCGGGCCCGUCGUGUCUUUCGGUGGACCUUGGG
CCGUCGGGCACGUACACGGGUGGCCGGGCGUUGGGGUGGAUCUUAGCCUC
CCCGGGCCAAUAUCGCUAGAGACAGCCGAUCUCCACGCGACCCC3′
(the RNA counterpart of SEQ ID NO: 13,
hereinafter SEQ ID NO: 16)
5′ACGGGGGUGGGGGGGGGGGGGUAUAUAAGGCCUGGGAUCCCACGUCC
CCG3′ (the RNA counterpart of SEQ ID NO: 14 i.e.
sequence upstream of SEQ ID NO: 13, hereinafter
SEQ ID NO: 17)
5′AUGGCCGCUCCCAACCGCGACCCUCCGGGAUACCGGUAUGCCGCGGC
CAU3′ (the RNA counterpart of SEQ ID NO: 15 i.e.
sequence downstream of SEQ ID NO: 13, hereinafter
SEQ ID NO: 18).

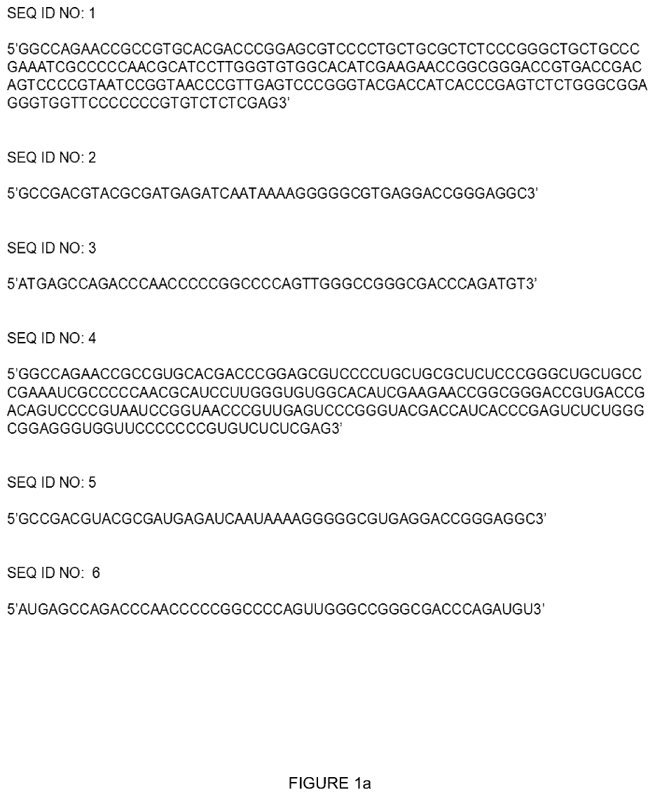

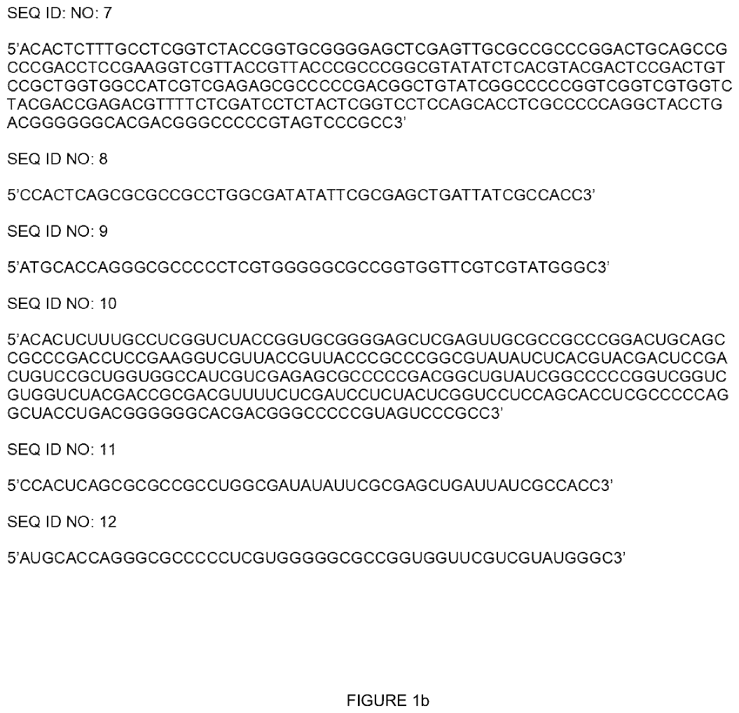

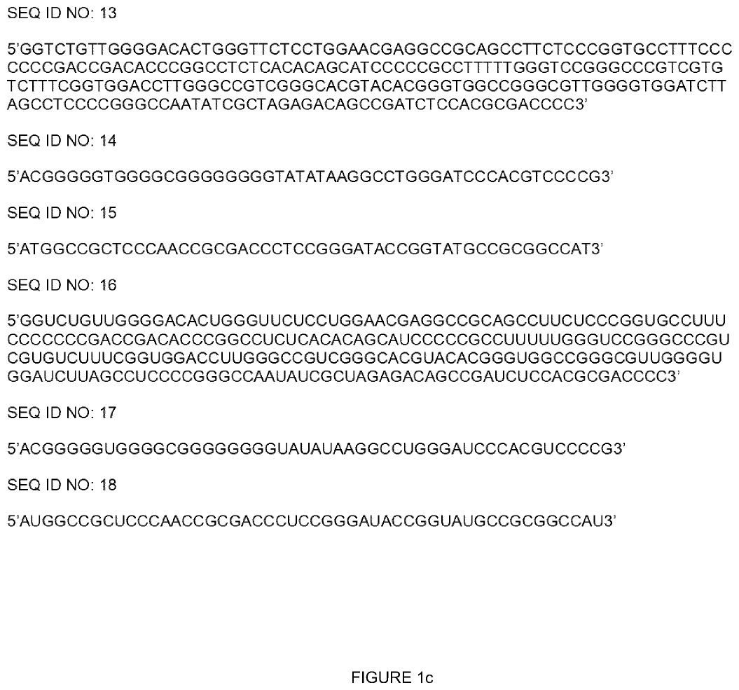

The representative nucleic acid sequences of the present invention are shown in FIG. 1a, FIG. 1b, and FIG. 1c. As would be understood by a person skilled in the art, the sequences recited above are merely representative and should not be construed as limiting the scope of the invention.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

The experimental results and data presented in this application shows that HSV1 US11 5′ leader sequence fused upstream of therapeutic payloads encoded within HSV1 can be used to greatly enhance transgene protein expression and improve the therapeutic efficacy of the virus. Similar results are expected from US11 5′ leader sequence of HSV or other HSV variants.

In view of the results presented in this application, the sequence of US11 5′ leader (i.e. SEQ ID NO: 1 or 4) can be employed and inserted in nucleic acids, vectors, gene delivery vehicles, recombinant DNA or RNA constructs, cell lines or compositions or kits comprising any of these, to enhance expression of any protein of interest. For instance, a vector or plasmid can be constructed, where the sequence of SEQ ID NO: 1 or 4, can be positioned upstream of a gene/cistron encoding a protein of interest (such as proteins capable of ameliorating or treating any medical condition, cellular defect, disease or disorder, or capable of providing any therapeutic effect). Additionally, a construct comprising SEQ ID NO: 1 or 4, can be used with a variety of cell lines in vivo or in vitro as desired. Accordingly, the following embodiments are proposed by the inventors.

### Nucleic Acids

The invention relates to a nucleic acid comprising SEQ ID NO: 1 or a fragment comprising at least 180 nucleotides thereof, or a sequence that is at least 90% identical to SEQ ID NO: 1, where the nucleic acid does not comprise SEQ ID NO: 2, SEQ ID NO: 3, or both, and where, the nucleotide sequence does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 1.

In some embodiments, the invention relates to a nucleic acid comprising SEQ ID NO: 4 or a fragment comprising at least 180 nucleotides thereof, or a sequence that is at least 90% identical to SEQ ID NO: 4, where the nucleic acid does not comprise SEQ ID NO: 5, SEQ ID NO: 6, or both, and where, the nucleotide sequence does not comprise a fragment of SEQ ID NO: 5, 6 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 4.

In some embodiments, the invention relates to a nucleic acid comprising SEQ ID NO: 7 or a fragment comprising at least 180 nucleotides thereof, or a sequence that is at least 90% identical to SEQ ID NO: 7, where the nucleic acid does not comprise SEQ ID NO: 8, SEQ ID NO: 9, or both, and where, the nucleotide sequence does not comprise a fragment of SEQ ID NO: 8, 9 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 7.

In some embodiments, the invention relates to a nucleic acid comprising SEQ ID NO: 10 or a fragment comprising at least 180 nucleotides thereof, or a sequence that is at least 90% identical to SEQ ID NO: 16, where the nucleic acid does not comprise SEQ ID NO: 11, SEQ ID NO: 12, or both, and where, the nucleotide sequence does not comprise a fragment of SEQ ID NO: 11, 12 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 10.

In some embodiments, the invention relates to a nucleic acid comprising SEQ ID NO: 13 or a fragment comprising at least 180 nucleotides thereof, or a sequence that is at least 90% identical to SEQ ID NO: 13, where the nucleic acid does not comprise SEQ ID NO: 14, SEQ ID NO: 15, or both, and where, the nucleotide sequence does not comprise a fragment of SEQ ID NO: 14, 15 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 13.

In some embodiments, the invention relates to a nucleic acid comprising SEQ ID NO: 16 or a fragment comprising at least 180 nucleotides thereof, or a sequence that is at least 90% identical to SEQ ID NO: 16, where the nucleic acid does not comprise SEQ ID NO: 17, SEQ ID NO: 18, or both, and where, the nucleotide sequence does not comprise a fragment of SEQ ID NO: 17, 18 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 16.

In an alternate embodiment, there is provided a nucleic acid comprising SEQ ID NO: 1 or a sequence that is at least 90% identical over the full length of SEQ ID NO: 1, where the nucleotide sequence does not comprise SEQ ID NO: 2, SEQ ID NO: 3, or both. Furthermore, the nucleic acid does not comprise a fragment of SEQ ID NO: 2, 3 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 1.

In an alternate embodiment, there is provided a nucleic acid comprising SEQ ID NO: 4 or a sequence that is at least 90% identical over the full length of SEQ ID NO: 4, where the nucleotide sequence does not comprise SEQ ID NO: 5, SEQ ID NO: 6, or both. Furthermore, the nucleic acid does not comprise a fragment of SEQ ID NO: 5, 6 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 4.

In an alternate embodiment, there is provided a nucleic acid comprising SEQ ID NO: 7 or a sequence that is at least 90% identical over the full length of SEQ ID NO: 7, where the nucleotide sequence does not comprise SEQ ID NO: 8, SEQ ID NO: 9, or both. Furthermore, the nucleic acid does not comprise a fragment of SEQ ID NO: 8, 9 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 7.

In an alternate embodiment, there is provided a nucleic acid comprising SEQ ID NO: 10 or a sequence that is at least 90% identical over the full length of SEQ ID NO: 10, where the nucleotide sequence does not comprise SEQ ID NO: 11, SEQ ID NO: 12, or both. Furthermore, the nucleic acid does not comprise a fragment of SEQ ID NO: 11, 12 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 10.

In an alternate embodiment, there is provided a nucleic acid comprising SEQ ID NO: 13 or a sequence that is at least 90% identical over the full length of SEQ ID NO: 13, where the nucleotide sequence does not comprise SEQ ID NO: 14, SEQ ID NO: 15, or both. Furthermore, the nucleic acid does not comprise a fragment of SEQ ID NO: 14, 15 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 13.

In an alternate embodiment, there is provided a nucleic acid comprising SEQ ID NO: 16 or a sequence that is at least 90% identical over the full length of SEQ ID NO: 16, where the nucleotide sequence does not comprise SEQ ID NO: 17, SEQ ID NO: 18, or both. Furthermore, the nucleic acid does not comprise a fragment of SEQ ID NO: 17, 18 or both which is at least 3, at least 5, at least 10, at least 15, at least 20, at least 25, at least 30, at least 35, at least 40, at least 45 or at least 50 nucleotide bases immediately continuous with the 5′ or 3′ to SEQ ID NO: 16.

As discussed earlier, the US11 gene sequence is highly conserved, and therefore, the nucleic acid identified by the inventors when isolated from the genome of HSV1 virus, is capable of exhibiting the desired effect i.e. increase protein expression by several fold, as compared to its expression in the absence of US11 5′ leader. The nucleotide sequences flanking SEQ ID NO: 1 are also highly conserved, and therefore, the embodiment described above clearly disclaims a sequence as it exists in nature i.e. 5′ SEQ ID NO: 2-SEQ ID NO: 1-SEQ ID NO: 3 3′, and only employs SEQ ID NO: 1 or a fragment or a sequence that is 90% identical to SEQ ID NO: 1. The nucleic acid does not comprise the sequences of SEQ ID NO: 2 or 3 or any fragments of SEQ ID NO: 2 or 3. Accordingly, the nucleic acid comprising SEQ ID NO: 1 can be engineered to insert a gene encoding a protein of interest along with other regulatory elements for increased protein expression. It is pertinent to note that increased protein production occurs with HSV1 infection.

As discussed earlier, the UL27 gene sequence is highly conserved, and therefore, the nucleic acid identified by the inventors when isolated from the genome of HSV1 virus, is capable of exhibiting the desired effect i.e. increase protein expression by several fold, as compared to its expression in the absence of UL27 5′ leader. The nucleotide sequences flanking SEQ ID NO: 7 are also highly conserved, and therefore, the embodiment described above clearly disclaims a sequence as it exists in nature i.e. 5′ SEQ ID NO: 8-SEQ ID NO: 7-SEQ ID NO: 9 3′, and only employs SEQ ID NO: 7 or a fragment or a sequence that is 90% identical to SEQ ID NO: 7. The nucleic acid does not comprise the sequences of SEQ ID NO: 8 or 9 or any fragments of SEQ ID NO: 8 or 9. Accordingly, the nucleic acid comprising SEQ ID NO: 7 can be engineered to insert a gene encoding a protein of interest along with other regulatory elements for increased protein expression. It is pertinent to note that increased protein production occurs with HSV1 infection.

As discussed earlier, the UL19 gene sequence is highly conserved, and therefore, the nucleic acid identified by the inventors when isolated from the genome of HSV1 virus, is capable of exhibiting the desired effect i.e. increase protein expression by several fold, as compared to its expression in the absence of UL19 5′ leader. The nucleotide sequences flanking SEQ ID NO: 13 are also highly conserved, and therefore, the embodiment described above clearly disclaims a sequence as it exists in nature i.e. 5′ SEQ ID NO: 14-SEQ ID NO: 13-SEQ ID NO: 15 3′, and only employs SEQ ID NO: 13 or a fragment or a sequence that is 90% identical to SEQ ID NO: 13. The nucleic acid does not comprise the sequences of SEQ ID NO: 14 or 15 or any fragments of SEQ ID NO: 14 or 15. Accordingly, the nucleic acid comprising SEQ ID NO: 13 can be engineered to insert a gene encoding a protein of interest along with other regulatory elements for increased protein expression. It is pertinent to note that increased protein production occurs with HSV1 infection.

Although, HSV1 virus is a dsDNA, the increase in protein expression occurs owing to increased ribosomal recruitment during translation which causes enhanced translation of the mRNA transcript, and therefore, the RNA counterpart of SEQ ID NO: 1 i.e. SEQ ID NO: 4, the RNS counterpart of SEQ ID NO: 7 i.e. SEQ ID NO: 10, or the RNA counterpart of SEQ ID NO: 13 i.e. SEQ ID NO: 16; is also capable of providing this desired effect. Therefore, in an alternate embodiment, there is provided a nucleic acid comprising SEQ ID NO: 4 i.e. the RNA counterpart of SEQ ID NO: 1, or a fragment comprising at least 180 nucleotides, or a sequence that is at least 90% identical to SEQ ID NO: 4, where the nucleic acid does not comprise SEQ ID NO: 5 (i.e. RNA counterpart of SEQ ID NO: 2), SEQ ID NO: 6 (i.e. RNA counterpart of SEQ ID NO: 3), or both. Similarly, in an alternate embodiment, there is provided a nucleic acid comprising SEQ ID NO: 10 i.e. the RNA counterpart of SEQ ID NO: 7, or a fragment comprising at least 180 nucleotides, or a sequence that is at least 90% identical to SEQ ID NO: 10, where the nucleic acid does not comprise SEQ ID NO: 11 (i.e. RNA counterpart of SEQ ID NO: 8), SEQ ID NO: 12 (i.e. RNA counterpart of SEQ ID NO: 9), or both. Similarly, in an alternate embodiment, there is provided a nucleic acid comprising SEQ ID NO: 16 i.e. the RNA counterpart of SEQ ID NO: 13, or a fragment comprising at least 180 nucleotides, or a sequence that is at least 90% identical to SEQ ID NO: 13, where the nucleic acid does not comprise SEQ ID NO: 17 (i.e. RNA counterpart of SEQ ID NO: 14), SEQ ID NO: 18 (i.e. RNA counterpart of SEQ ID NO: 15), or both.

In an alternate embodiment, there is provided a sequence that is at least 90% identical to SEQ ID NO: 4, or a fragment comprising at least 180 nucleotides where the nucleic acid does not comprise SEQ ID NO: 5, SEQ ID NO: 6, or both. The nucleic acid described above does not comprise a fragment of SEQ ID NO: 5, 6 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 4. Thus, RNA nucleic acid or RNA counterpart of the nucleic acid defined above is also contemplated.

In an embodiment of the invention, the nucleic acid does not comprise a fragment of at least 3, at least 5, at least 10, at least 15, at least 20, at least 25, at least 30, at least 35, at least 40, at least 45 or at least 50 nucleotide bases of SEQ ID NO: 2 immediately continuous with the 5′ to SEQ ID NO: 1; or wherein the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 3 immediately continuous with the 3′ to SEQ ID NO: 1. An alternate embodiment comprising the RNA counterpart of SEQ ID NO: 1, 2 and 3 (i.e. SEQ ID NO: 4, 5 and 6) is also contemplated.

In an alternate embodiment, the nucleic acid does not comprise a fragment of at least 3, at least 5, at least 10, at least 15, at least 20, at least 25, at least 30, at least 35, at least 40, at least 45 or at least 50 nucleotide bases of SEQ ID NO: 2 immediately continuous with the 5′ to SEQ ID NO: 1; and wherein the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 3 immediately continuous with the 3′ to SEQ ID NO: 1. An alternate embodiment comprising the RNA counterpart of SEQ ID NOs: 1, 2 and 3 (i.e. SEQ ID NOs: 4, 5 and 6) is also contemplated.

In an alternate embodiment, the nucleic acid does not comprise the entire length of SEQ ID NO: 2 or the entire length of SEQ ID NO: 3 immediately continuous with the 5′ or 3′ to SEQ ID NO: 1. An alternate embodiment comprising the RNA counterpart of SEQ ID NOs: 1, 2 and 3 (i.e. SEQ ID NOs: 4, 5 and 6) is also contemplated.

Alternate embodiments with SEQ ID NO: 7, 13, or their RNA counterparts i.e. SEQ ID NO: 10, or 16 are also contemplated. In each embodiment with SEQ ID NO: 7, the nucleic acid does not comprise SEQ ID NO: 8, 9 or both. In each embodiment with SEQ ID NO: 10, the nucleic acid does not comprise SEQ ID NO: 11, 12 or both. In each embodiment with SEQ ID NO: 13, the nucleic acid does not comprise SEQ ID NO: 14, 15 or both. In each embodiment with SEQ ID NO: 16, the nucleic acid does not comprise SEQ ID NO: 17, 18 or both.

In an embodiment of the invention, the nucleotide sequence or the nucleic acid does not comprise at least 250, 500, 1000 or more continuous nucleotides of Human herpesvirus 1 strain KOS, complete genome defined by NCBI Accession number: JQ673480.1 GI: 380776962 or by Accession number JQ780693.1 GI: 384597744, or a sequence which is 95% identical thereto.

In an embodiment of the invention, a second nucleic acid consisting of the nucleic acid of claim 1 is also contemplated. In an alternate embodiment, the nucleic acid could be a synthetic or recombinant nucleic acid comprising the sequence of SEQ ID NO: 1. The nucleic acid could also be in the form of an expression vector or a plasmid, where the expression vector or plasmid is heterologous to HSV1 and drives production of a protein of interest.

In some embodiments, the nucleic acid additionally comprises a promoter, a nucleotide sequence encoding a protein of interest, one or more regulatory sequences, one or more restriction endonuclease or cloning sites, one or more polyadenylation sites or a combination of all of them, where at least one or more of the promoter, nucleotide sequence encoding a protein of interest, one or more restriction endonuclease or cloning sites, one or more polyadenylation sites, or a combination of them is heterologous to HSV1.

Some non-limiting examples of promoters that can be fused with SEQ ID NO: 1, 4, 7, 10, 13, or 16 include: CMV, EF1a, CAG, PGK, TRE, U6, and UAS. Some non-limiting examples of proteins of interest include The nucleotides sequence encoding a protein of interest can be selected from GM-CSF, TNF-a, p53, GFP, chloramphenicol acetyltransferase (CAT), SMN Protein, lipoprotein lipase, Tat protein, Ebola glycoprotein, SARS-COV-2 Spike(S) protein, Cytokines, Ovalbumin, retinoid isomerohydrolase RPE65, insulin, SIV Env and Nef antigens OR Gag, Env and a Tat-Rev-Nef fusion protein, Viral Antigens, Cyclin G1, immunogenic proteins, immunomodulatory proteins, or cell-regulatory proteins. The protein of interest may be a reporter protein, a cell regulatory protein or a cytotoxic protein.

The one or more regulatory sequences can be selected from any known promoters, enhancers, silencers, transcription factors, coactivators, or operators. The one or more restriction endonuclease or cloning sites are palindromic sequences that are recognizable by restriction endonucleases. The one or more polyadenylation sites could be a stretch of Adenine bases or SV40 site or any other site known in the art.

In the embodiment recited above, the promoter is positioned upstream or 5′ to sequence SEQ ID NO: 1 and the nucleotide sequence encoding a protein of interest is positioned downstream or 3′ to sequence SEQ ID NO: 1. In some embodiments, the promoter is positioned immediately upstream to sequence SEQ ID NO: 1 or its RNA counterpart and the nucleotide sequence encoding a protein of interest is positioned immediately downstream to sequence SEQ ID NO: 1 or its RNA counterpart.

In some embodiments, the nucleic acid may be linear whereas in alternate embodiments the nucleic acid may be circular. The nucleic acid of any of the above embodiments is capable of increasing translation of the nucleotide sequence encoding a protein of interest compared to its translation in the absence of SEQ ID NO: 1 or 4.

In an embodiment of the invention, there is provided a nucleic acid consisting of a nucleotide sequence of SEQ ID NO: 1 or SEQ ID NO: 4. In an alternate embodiment, the nucleic acid may comprise a sequence of SEQ ID NO: 1, wherein the sequence of SEQ ID NO: 1 is isolated from the genome of HSV1. The nucleic acid recited herein before, wherein the nucleic acid does not comprise the sequence of UL9 gene downstream or 3′ to SEQ ID NO: 1 or does not comprise the sequence of UL12 gene upstream or 5′ to SEQ ID NO: 1, as found in natural HSV1 genome. Additionally, the sequence SEQ ID NO: 1 claimed in the present invention does not comprise the sequence complementary to the sequence of US10 gene immediately continuous downstream or 3′ to SEQ ID NO: 1. An alternate embodiment, covering the mRNA counterpart of SEQ ID NO: 1 (i.e. SEQ ID NO: 4) is also contemplated.

In other embodiments, the nucleic acid sequence does not comprise a fragment of at least 3, at least 5, at least 10, at least 15, at least 20, at least 25, at least 30, at least 35, at least 40, at least 45 or at least 50 nucleotide bases of SEQ ID NO: 2 thereof immediately continuous with the 5′ to SEQ ID NO: 1; or wherein the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 3 thereof immediately continuous with the 3′ to SEQ ID NO: 1. An alternate embodiment covering counterpart sequences i.e. SEQ ID NO: 4, 5 and 6 is also contemplated.

In an alternate embodiment, the nucleic acid nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 2 thereof immediately continuous with the 5′ to SEQ ID NO: 1; and the nucleotide sequence does not further comprise a fragment of at least 3, at least 5, at least 10, at least 15, at least 20, at least 25, at least 30, at least 35, at least 40, at least 45 or at least 50 nucleotide bases of SEQ ID NO: 3 thereof immediately continuous with the 3′ to SEQ ID NO: 1. An alternate embodiment covering counterpart sequences i.e. SEQ ID NO: 4, 5 and 6 is also contemplated.

In some embodiments, the nucleic acid does not comprise SEQ ID NO: 2 immediately continuous with the 5′ or 3′ to SEQ ID NO: 1 or wherein the nucleic acid does not comprise SEQ ID NO: 2 immediately continuous with the 5′ to SEQ ID NO: 1. In an alternate embodiment, the nucleic acid does not comprise SEQ ID NO: 3 immediately continuous with the 5′ or 3′ to SEQ ID NO: 1 or where the nucleic acid does not comprise SEQ ID NO: 3 immediately continuous with the 3′ to SEQ ID NO: 1. An alternate embodiment covering counterpart sequences i.e. SEQ ID NO: 4, 5 and 6 is also contemplated.

In an embodiment of the invention, there is provided a synthetic/recombinant nucleic acid comprising a nucleic acid of SEQ ID NO: 1 or SEQ ID NO: 4 (for mRNA embodiment). This nucleic acid does not comprise a fragment of at least 3, at least 5, at least 10, at least 15, at least 20, at least 25, at least 30, at least 35, at least 40, at least 45 or at least 50 nucleotide bases of SEQ ID NO: 2 thereof immediately continuous with the 5′ to SEQ ID NO: 1; or does not comprise a fragment of at least 3, at least 5, at least 10, at least 15, at least 20, at least 25, at least 30, at least 35, at least 40, at least 45 or at least 50 nucleotide bases of SEQ ID NO: 5 thereof immediately continuous with the 5′ to SEQ ID NO: 4; or where the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 3 thereof immediately continuous with the 3′ to SEQ ID NO: 1, or where the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 6 thereof immediately continuous with the 3′ to SEQ ID NO: 4.

In an embodiment of the invention, there is provided a synthetic/recombinant nucleic acid comprising a nucleic acid of SEQ ID NO: 1 or SEQ ID NO: 4 (for mRNA embodiment). This nucleic acid does not comprise a fragment of at least 3, at least 5, at least 10, at least 15, at least 20, at least 25, at least 30, at least 35, at least 40, at least 45 or at least 50 nucleotide bases of SEQ ID NO: 2 thereof immediately continuous with the 5′ to SEQ ID NO: 1; and where the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 3 thereof immediately continuous with the 3′ to SEQ ID NO: 1; or where the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 5 thereof immediately continuous with the 5′ to SEQ ID NO: 4; and where the nucleotide sequence does not comprise a fragment of at least 3, at least 5, at least 10, at least 15, at least 20, at least 25, at least 30, at least 35, at least 40, at least 45 or at least 50 nucleotide bases of SEQ ID NO: 6 thereof immediately continuous with the 3′ to SEQ ID NO: 4.

In any of the above recited embodiments, the nucleic acid may further comprise a promoter or a gene encoding a protein of interest, or both. The promoter may be positioned upstream or 5′ to sequence SEQ ID NO: 1 or SEQ ID NO: 4. The gene encoding a protein of interest is positioned downstream or 3′ to sequence SEQ ID NO: 1 or SEQ ID NO: 4. In an alternate embodiment, the promoter may be positioned 5′ to sequence SEQ ID NO: 1 or SEQ ID NO: 4 and the gene encoding a protein of interest is positioned 3′ to sequence SEQ ID NO: 1 or SEQ ID NO: 4. In some embodiments, the promoter is positioned immediately upstream to sequence SEQ ID NO: 1 or its RNA counterpart and the nucleotide sequence encoding a protein of interest is positioned immediately downstream to sequence SEQ ID NO: 1 or its RNA counterpart.

In some embodiments, the nucleic acid may be a single stranded RNA, a single stranded DNA, a double stranded RNA, or a double stranded DNA. The nucleic acid comprising sequence SEQ ID NO: 1 gets transcribed to mRNA which has an RNA sequence corresponding to SEQ ID NO: 1 i.e. sequence of SEQ ID NO: 4.

In some embodiments, the nucleic acids recited in this application could be in the form of a concatemer i.e. the nucleic acid may contain several repetitive units of SEQ ID NO: 1 or where SEQ ID NO: 1 is repeated at least two or more times.

The nucleic acids recited hereinbefore are capable of enhancing translation and therefore act as translation enhancers. More specifically, the transcription of SEQ ID NO 1 comprised in the nucleic acid increases translation of the gene encoding a protein of interest when positioned downstream or 3′ to SEQ ID NO: 1, compared to its translation in the absence of SEQ ID NO: 1. A similar embodiment containing the RNA counterpart of SEQ ID NO: 1 i.e. SEQ ID NO: 4 is also feasible.

In some embodiments, the nucleic acid is a transgene or where the nucleic acid is inserted in a gene delivery vehicle. A few non-limiting examples of gene delivery vehicles include, a plasmid, a vector, a recombinant DNA or RNA construct, or an expression cassette or a nanoparticle, which could be a lipid nanoparticle.

In an embodiment of the invention, the nucleic acid comprises additional components. For instance, the nucleic acid may comprise a sequence of SEQ ID NO: 1; and a restriction endonuclease site for a gene encoding a protein of interest. In an alternate embodiment, the nucleic acid comprises a sequence of SEQ ID NO: 1; a restriction endonuclease site for a gene encoding a protein of interest; and a restriction endonuclease site for a promoter, a regulatory element or both. The restriction endonuclease site for a gene encoding a protein of interest may be positioned downstream or 3′ to SEQ ID NO: 1. The restriction endonuclease site for a promoter or regulatory element is positioned upstream or 5′ to SEQ ID NO: 1. In an alternate embodiment, the restriction endonuclease site for a gene encoding a protein of interest is positioned immediately continuous with the 3′ to SEQ ID NO: 1 and the restriction endonuclease site for a promoter or regulatory element is positioned immediately continuous with the 5′ to SEQ ID NO: 1. Transcription of the sequence of SEQ ID NO: 1, increases translation of the gene encoding the protein of interest as compared to its translation in the absence of SEQ ID NO: 1. More specifically, wherein translation of the sequence encoding the protein of interest in the presence of SEQ ID NO: 1 increases expression, synthesis, or production of the protein of interest by several fold compared to its expression, synthesis, or production in the absence of SEQ ID NO: 1. A similar embodiment containing the RNA counterpart of SEQ ID NO: 1 i.e. SEQ ID NO: 4 is also capable of providing similar results.

In some embodiments, the nucleic acid recited above may additionally comprise a polyadenylation sequence, wherein the polyadenylation sequence is downstream or 3′ to the sequence encoding a protein of interest. In an alternate embodiment, the polyadenylation sequence is immediately continuous with the 3′ to the sequence encoding a protein of interest. Without wishing to be limiting, the polyadenylation sequence can be selected from selected from any polyadenylation sequence known in the art. In some embodiments, the polyadenylation sequence is SV40 polyadenylation sequence.

The gene encoding a protein of interest may be a cistron, which may encode for any of the following proteins: GM-CSF, TNF-α, p53, GFP, chloramphenicol acetyltransferase (CAT), GM-CSF, SMN Protein, lipoprotein lipase, Tat protein, Ebola glycoprotein, SARS-COV-2 Spike(S) protein, Cytokines, Ovalbumin, retinoid isomerohydrolase RPE65, insulin, SIV Env and Nef antigens OR Gag, Env and a Tat-Rev-Nef fusion protein, Viral Antigens, Cyclin G1, immunogenic proteins, immunomodulatory proteins, or cell-regulatory proteins. The protein of interest may be a reporter protein, a cell regulatory protein or a cytotoxic protein.

In some embodiments, the cistron encodes for GM-CSF protein or TNF-α protein. In some embodiments, the cistron encodes for a protein that targets/cistron is selected to target a specific cell line. Some non-limiting examples could be a carcinoma cell line, melanoma cell line, neuronal cell line, epithelial cell line, or lymphocyte cell line.

In the above recited embodiments, the regulatory element promoters or translation enhancers used may be cis acting, non-limiting examples of which are provided above.

In an embodiment of the invention, there is provided a nucleic acid comprising: a promoter; a sequence of SEQ ID NO: 1 or 4; and a gene encoding a protein of interest. Therefore, in some embodiments, the nucleic acid may comprise SEQ ID NO: 1 or 4, a CMV promoter and gene encoding for GM-CSF. In some alternate embodiments, the nucleic acid may comprise SEQ ID NO: 1 or 4, a CMV promoter and gene encoding for TNF-α. The promoter i.e. CMV, is positioned upstream or 5′ to the sequence SEQ ID NO: 1 or 4. The gene encoding a protein of interest i.e. GM-CSF is positioned downstream or 3′ to the sequence SEQ ID NO: 1 or 4. In an alternate embodiment, the promoter may be positioned 5′ to the sequence SEQ ID NO: 1 or 4, and the gene encoding a protein of interest may be positioned 3′ to the sequence SEQ ID NO: 1 or 4. The promoter may be selected from any of the promoter listed hereinbefore. The gene encoding a protein of interest may be a cistron, which may be selected from the examples listed hereinbefore. The cistron selected may encode for a protein of interest that targets a specific cell line, some non-limiting examples of which are provided in this application. As noted earlier, translation of the sequence gene the protein of interest in the presence of SEQ ID NO: 1 or 4 increases expression, synthesis, or production of the protein of interest by several fold compared to its expression, synthesis, or production in the absence of SEQ ID NO: 1 or 4. In some embodiments, the increase in protein expression, synthesis, or production is 0.5 fold, 1 fold, 2 fold, 3 fold, 4 fold, 5 fold, 6 fold, 7 fold, or 8 fold. The increases in protein expression, synthesis or production usually occurs with HSV1 infection.

In an embodiment of the invention, the nucleic acid may comprise, a CMV promoter; a sequence of SEQ ID NO: 1 or 4; and a gene encoding for GM-CSF protein. In an alternate embodiment, the nucleic acid may comprise, a CMV promoter; a sequence of SEQ ID NO: 1 or 4; and a gene encoding for TNF-a protein. The CMV promoter is positioned upstream or 5′ to the sequence of SEQ ID NO: 1 or 4. The gene encoding for GM-CSF protein is positioned 3′ to sequence SEQ ID NO: 1 or 4. Transcription of the sequence of SEQ ID NO: 1 increases translation of the gene encoding for GM-CSF protein by several fold i.e. 0.5 fold, 1 fold, 2 fold, 3 fold, 4 fold, 5 fold, 6 fold, 7 fold, or 8 fold, compared to its translation in the absence of SEQ ID NO: 1 or 4.

In some embodiments, the nucleic acid may be inserted in a gene delivery vehicle such as a plasmid, a vector, a recombinant DNA or RNA construct, or an expression cassette. Some non-limiting examples of vector that can be employed included a viral vector, a live-viral vector, an oncolytic viral vector, an attenuated viral vector, a recombinant vector or an amplicon vector. In some alternate embodiments, the nucleic acid may be inserted in incorporated in a nanoparticle, or a lipid nanoparticle, or in HSV1 or HSV1 based gene delivery vehicles or constructs. When the nucleic acid is inserted anywhere in HSV1 or HSV1 based constructs, the nucleic acid may be inserted at any site or restriction endonuclease site of HSV1 genome. In an alternate embodiment, the nucleic acid may be inserted at the tk locus of HSV1.

The nucleic acid inserted in the HSV1 based gene delivery vehicle or constructs, may be used for administration to a patient in need thereof, where the patient is pre-infected with HSV1 virus prior to administration of the HSV1 virus based delivery vehicle. Alternatively, the nucleic acid is inserted in HSV1 live viral vector or an oncolytic HSV1 vector, and the live viral vector is used for administration to a patient in need thereof, which eliminates the requirement of pre-infection. The administration of nucleic acid increases protein expression, synthesis, or production, however, the increased production is only evident in cells of the patient which are pre-infected with HSV1 virus.

In an embodiment of the invention, the nucleic acid may be inserted in HSV1 virus, wherein the virus is modified to include an endonuclease site for a promoter or a regulatory element; and an endonuclease site for a sequence encoding a protein of interest. The promoters, regulatory elements and downstream cistrons may be selected from the examples provided hereinbefore. HSV1 virus delivers the nucleic acid containing SEQ ID NO: 1 or 4 to the cells and causes increase in protein production of the downstream cistron by several fold. This can be extremely helpful for targeting specific cell lines or for treating a medical condition, cellular defect or disease as large quantities of protein of interest could be delivered to a patient in need thereof. As noted earlier, the increase in protein production requires pre-infection with HSV1 virus and the increase in protein production is several fold compared to its production in the absence of SEQ ID NO: 1.g

The nucleic acids recited hereinbefore can be utilized as a 5′ UTR sequence or a leader sequence or a cis-acting regulatory element, wherein the nucleic acid possibly has low folding free energy compared to other 5′ UTR sequences of HSV1 genome.

For all the above embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Vectors and Other Constructs

In an embodiment of the invention, there is provided a vector comprising the SEQ ID NO: 1 or a fragment comprising at least 180 nucleotides, or a sequence that is at least 90% identical to SEQ ID NO: 1, or; a sequence that is at least 90% identical to SEQ ID NO: 1, or a fragment comprising at least 180 nucleotides and a promoter, a nucleotide sequence encoding a protein of interest, one or more regulatory sequences, one or more restriction endonuclease or cloning sites, and one or more polyadenylation sites or any combination thereof. An alternate embodiment comprising the RNA counterpart of SEQ ID NOs: 1, 2 and 3 (i.e. SEQ ID NOs: 4, 5 and 6) is also contemplated.

As noted hereinbefore, both viral and non-viral constructs can be used to insert the US11 5′ leader sequence. In some embodiment, commercially available vectors can be modified to insert the US11 5′ leader to increase the efficacy of existing gene therapies. Some examples include T-vec (Amgen), HSV-1716 (Virttu Therapeutics-acquired by Sorrento), Immvira, Virogin, Replimune, Treovir, J&J, BeneVir, and Oncorus.

In an embodiment, the vector is a viral vector recombinantly transformed with a heterologous nucleic acid comprising: SEQ ID NO: 1 or a fragment comprising at least 180 nucleotides, or a sequence that is at least 90% identical to SEQ ID NO: 1, or; a sequence that is at least 90% identical to SEQ ID NO: 1, or a fragment comprising at least 180 nucleotides, and a promoter, a nucleotide sequence encoding a protein of interest, one or more regulatory sequences, one or more restriction endonuclease or cloning sites, and one or more polyadenylation sites or any combination thereof. An alternate embodiment comprising the RNA counterpart of SEQ ID NOs: 1, 2 and 3 (i.e. SEQ ID NOs: 4, 5 and 6) is also contemplated.

The viral vector comprises at least one of SEQ ID NO: 1, or a fragment of SEQ ID NO: 1, or a sequence that is at least 90% identical to SEQ ID NO: 1, the fragment comprising at least 180 nucleotides, the promoter, the nucleotide sequence encoding a protein of interest, the one or more regulatory sequences, the one or more restriction endonuclease or cloning sites, the one or more polyadenylation sites, or any combination is heterologous to the viral vector. The viral vector could be live, attenuated, oncolytic or a combination of any of these. In an embodiment, the viral vector may be an HSV1 viral vector, and more specifically, the HSV1 viral vector is HSV1. An alternate embodiment comprising the RNA counterpart of SEQ ID NOs: 1, 2 and 3 (i.e. SEQ ID NOs: 4, 5 and 6) is also contemplated.

In an embodiment of the invention, there is provided a vector comprising any of the nucleic acids recited hereinbefore. In some embodiments the vector comprises, an endonuclease site for a promoter or a regulatory element, a nucleic acid comprising SEQ ID NO: 1 or 4, and an endonuclease site for a gene encoding a protein of interest. The nucleic acid recited here comprises SEQ ID NO: 1 or a sequence that is at least 90% identical thereto over the full length of SEQ ID NO: 1, wherein the nucleotide sequence does not comprise SEQ ID NO: 2, SEQ ID NO: 3, or both, and wherein, the nucleotide sequence does not comprise a fragment of SEQ ID NO: 2, 3 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 1. In an alternate embodiment, the nucleic acid recited here comprises SEQ ID NO: 4 or a sequence that is at least 90% identical thereto over the full length of SEQ ID NO: 4, wherein the nucleotide sequence does not comprise SEQ ID NO: 5, SEQ ID NO: 6, or both, and wherein, the nucleotide sequence does not comprise a fragment of SEQ ID NO: 5, 6 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 4. The nucleic acid additionally comprises a gene encoding a protein of interest, and optionally a promoter, regulatory element or translation enhancer. Transcription of the SEQ ID NO: 1 or 4, increases translation of the gene encoding the protein of interest, thereby inducing increased protein expression, synthesis or production in a cell, compared to its translation in the absence of SEQ ID NO: 1 or 4.

In the embodiments of the invention, the vector may comprise any of the other nucleic acid embodiments recited above. Additionally, the nucleic acid may comprise any regulatory elements, translation enhancers, or promoters, non-limiting examples of which are provided above.

The vectors recited hereinbefore, may be selected from the following non-limiting examples: a viral vector, a live-viral vector, an oncolytic viral vector, an attenuated viral vector, a recombinant vector or an amplicon vector. In some embodiments, the vector is constructed using HSV1 virus, wherein the nucleic acid is inserted anywhere on the HSV1 genome, or at any restriction site or at the tk locus or any other endonuclease site on the HSV1 genome.

In some alternate embodiments, the nucleic acid may be inserted in a gene delivery vehicle, some non-limiting examples of which are a plasmid, an expression cassette, a live virus, a DNA or RNA construct or recombinant nucleotide construct, an intron-less open reading frame, a nanoparticle, or a lipid nanoparticle. In some embodiments, the gene delivery vehicles recited above may be HSV1 based.

For all the above vector-based embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Cell Lines

In an embodiment of the invention, there is provided a cell comprising the nucleic acid (comprising SEQ ID NO: 1 or 4) or a vector (comprising the SEQ ID NO: 1 or 4). The cell may comprise a nucleic acid, or a vector, either alone or in combination, comprising SEQ ID NO: 1 or a fragment comprising at least 180 nucleotides, or a sequence that is at least 90% identical to SEQ ID NO: 1, where the nucleic acid does not comprise SEQ ID NO: 2, SEQ ID NO: 3, or both. In an alternate embodiment, there is provided a composition comprising a sequence that is at least 90% identical to SEQ ID NO: 1, or a fragment comprising at least 180 nucleotides where the nucleic acid does not comprise SEQ ID NO: 2, SEQ ID NO: 3, or both. As noted earlier, the nucleic acid described above does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 1. An alternate embodiment comprising the RNA counterpart of SEQ ID NOs: 1, 2 and 3 (i.e. SEQ ID NOs: 4, 5 and 6) is also contemplated.

The cell could be a mammalian cell, or specifically a cancer cell. In the presence of the 5′ leader sequence, the cell exhibits an increase in protein expression, synthesis, or production of more than about 0.5 fold, about 1 fold, about 2 fold, about 3 fold, about 4 fold, about 5 fold, about 6 fold, about 7 fold, about 8 fold or more compared to an identical control cell which lacks SEQ ID NO: 1. This is consistent with the experimental results shown above. An alternate embodiment comprising the RNA counterpart of SEQ ID NOs: 1, 2 and 3 (i.e. SEQ ID NOs: 4, 5 and 6) is also contemplated.

In the embodiments recited above, the sequence SEQ ID NO: 1 may be inserted anywhere on the HSV1 genome, or at any restriction site or at the tk locus of HSV1. In an alternate embodiment, the sequence SEQ ID NO: 1 could be inserted at any other site of HSV1. Therefore, the nucleic acid, vector or cell comprising the sequence SEQ ID NO: 1 could have the sequence specifically inserted at the tk locus of HSV1 or optionally at any other restriction endonuclease site. However, the desired effect of enhanced protein expression is only observed in cells which are pre-infected with HSV1 virus.

In an alternate embodiment, the cell may comprise any of the nucleic acid embodiments recited hereinbefore and a pharmaceutically acceptable carrier or excipient. For instance, the cell may comprise a nucleic acid comprising SEQ ID NO: 1 or 4, and a pharmaceutically acceptable carrier or excipient. The nucleic acid may comprise SEQ ID NO: 1 or a sequence that is at least 90% identical thereto over the full length of SEQ ID NO: 1, wherein the nucleotide sequence does not comprise SEQ ID NO: 2, SEQ ID NO: 3, or both, and wherein, the nucleotide sequence does not comprise a fragment of SEQ ID NO: 2, 3 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 1. In an alternate embodiment, the nucleic acid recited here comprises SEQ ID NO: 4 or a sequence that is at least 90% identical thereto over the full length of SEQ ID NO: 4, wherein the nucleotide sequence does not comprise SEQ ID NO: 5, SEQ ID NO: 6, or both, and wherein, the nucleotide sequence does not comprise a fragment of SEQ ID NO: 5, 6 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 4. The nucleic acid additionally comprises a gene encoding a protein of interest, and optionally a promoter, regulatory element or translation enhancer. Transcription of the SEQ ID NO: 1 or 4, increases translation of the gene encoding the protein of interest, thereby inducing increased protein expression, synthesis or production in a cell, compared to its translation in the absence of SEQ ID NO: 1 or 4.

For all the above cell-lines embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Compositions

In an embodiment of the invention, there is provided a composition comprising a nucleic acid, vector or cell, either alone or in combination, comprising SEQ ID NO: 1 or a fragment comprising at least 180 nucleotides, or a sequence that is at least 90% identical to SEQ ID NO: 1, where the nucleic acid does not comprise SEQ ID NO: 2, SEQ ID NO: 3, or both. In an alternate embodiment, there is provided a composition comprising a sequence that is at least 90% identical to SEQ ID NO: 1, or a fragment comprising at least 180 nucleotides where the nucleic acid does not comprise SEQ ID NO: 2, SEQ ID NO: 3, or both. As noted earlier, the nucleic acid described above does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases immediately continuous with the 5′ or 3′ ends of SEQ ID NO: 1. An alternate embodiment comprising the RNA counterpart of SEQ ID NOs: 1, 2 and 3 (i.e. SEQ ID NOs: 4, 5 and 6) is also contemplated.

In an alternate embodiment, the composition may comprise any of the nucleotide embodiments recited hereinbefore and a pharmaceutically acceptable carrier or excipient. For instance, the composition may comprise a nucleic acid comprising SEQ ID NO: 1 or 4, and a pharmaceutically acceptable carrier or excipient. The nucleic acid may comprise SEQ ID NO: 1 or a sequence that is at least 90% identical thereto over the full length of SEQ ID NO: 1, wherein the nucleotide sequence does not comprise SEQ ID NO: 2, SEQ ID NO: 3, or both, and wherein, the nucleotide sequence does not comprise a fragment of SEQ ID NO: 2, 3 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 1. In an alternate embodiment, the nucleic acid recited here comprises SEQ ID NO: 4 or a sequence that is at least 90% identical thereto over the full length of SEQ ID NO: 4, wherein the nucleotide sequence does not comprise SEQ ID NO: 5, SEQ ID NO: 6, or both, and wherein, the nucleotide sequence does not comprise a fragment of SEQ ID NO: 5, 6 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 4. The nucleic acid additionally comprises a gene encoding a protein of interest, and optionally a promoter, regulatory element or translation enhancer. Transcription of the SEQ ID NO: 1 or 4, increases translation of the gene encoding the protein of interest, thereby inducing increased protein expression, synthesis or production in a cell, compared to its translation in the absence of SEQ ID NO: 1 or 4.

Additionally, the composition may comprise a nucleic acid is provided in the form of a plasmid, a vector, a recombinant DNA or RNA construct, a gene delivery vehicle, a nanoparticle, and one or more pharmaceutically acceptable excipients.

The composition may be in the form of a capsule, an injectable, a topical cream or a powder. In some embodiments, the composition may be in the form of an injectable.

In other embodiments, the composition may comprise the other nucleic acid, vector and cell based embodiments recited above either alone or in combination, optionally comprising one or more pharmaceutically acceptable carriers, excipients, or diluents.

The pharmaceutically acceptable carriers, excipients or diluents may be selected from the non-limiting examples provided above.

For all the above composition embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Kits

In an embodiment of the invention, there is provided a kit comprising the nucleic acid embodiments recited hereinbefore (comprising SEQ ID NO: 1 or 4), the vector embodiments recited above (i.e. vector comprising SEQ ID NO: 1 or 4), or any of the cellular embodiments recited above (i.e. cells comprising SEQ ID NO: 1 or 4), along with one or more pharmaceutically acceptable carriers, excipients, or diluents, or one or more buffers, wash or cell culture media, or one or more vessels for containing any of the above, or instructions for expressing or augmenting expression of a protein of interest, or instructions for using any component of the kit, or any combination of the above components.

In an alternate embodiment, the kit may comprise any of the nucleotide embodiments recited hereinbefore and a pharmaceutically acceptable carrier or excipient. For instance, the composition may comprise a nucleic acid comprising SEQ ID NO: 1 or 4, along with one or more pharmaceutically acceptable carriers, excipients, or diluents, or one or more buffers, wash or cell culture media, or one or more vessels for containing any of the above, or instructions for expressing or augmenting expression of a protein of interest, or instructions for using any component of the kit, or any combination of the above components. The nucleic acid additionally comprises a gene encoding a protein of interest, and optionally a promoter, regulatory element or translation enhancer. The nucleic acid may comprise SEQ ID NO: 1 or a sequence that is at least 90% identical thereto over the full length of SEQ ID NO: 1, wherein the nucleotide sequence does not comprise SEQ ID NO: 2, SEQ ID NO: 3, or both, and wherein, the nucleotide sequence does not comprise a fragment of SEQ ID NO: 2, 3 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 1. In an alternate embodiment, the nucleic acid recited here comprises SEQ ID NO: 4 or a sequence that is at least 90% identical thereto over the full length of SEQ ID NO: 4, wherein the nucleotide sequence does not comprise SEQ ID NO: 5, SEQ ID NO: 6, or both, and wherein, the nucleotide sequence does not comprise a fragment of SEQ ID NO: 5, 6 or both which is at least 3, at least 5, at least 10, at least 15, at least 20, at least 25, at least 30, at least 35, at least 40, at least 45 or at least 50 nucleotide bases thereof immediately continuous with the 5′ or 3′ to SEQ ID NO: 4. The nucleic may additionally comprise a gene encoding a protein of interest, and optionally a promoter or a regulatory element. Transcription of the SEQ ID NO: 1 or 4, increases translation of the gene encoding the protein of interest, thereby inducing increased protein expression, synthesis or production in a cell, compared to its translation in the absence of SEQ ID NO: 1 or 4.

Additionally, the kit may comprise a nucleic acid is provided in the form of a plasmid, a vector, a recombinant DNA or RNA construct, a gene delivery vehicle, a nanoparticle, along with one or more pharmaceutically acceptable carriers, excipients, or diluents, or one or more buffers, wash or cell culture media, or one or more vessels for containing any of the above, or instructions for expressing or augmenting expression of a protein of interest, or instructions for using any component of the kit, or any combination of the above components.

The kit may comprise a composition with SEQ ID NO: 1 or 4, wherein the composition may be in the form of a capsule, an injectable, a topical cream or a powder form or any other form known in the art. In some embodiments, the composition may be in the form of an injectable.

In other embodiments, the kit may comprise the nucleic acid, vector and cell based embodiments recited above either alone or in combination, optionally comprising one or more pharmaceutically acceptable carriers, excipients, or diluents.

The kit may additionally comprise a container means which may include at least one vial, test tube, flask, bottle, syringe or other container means, into which the nucleic acid, vector, cells, compositions and/or or other materials may be placed and in embodiments a kit will include instructions regarding the use of the materials comprised in the kit.

For all the above kit-based embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Method of Producing Protein of Interest or Increasing Expression

In an embodiment of the invention, there is provided a method of producing a protein of interest in a cell comprising, wherein the method comprises administering a nucleic acid to the cell. The nucleic acid being administered comprises a promoter, SEQ ID NO: 1 and a sequence encoding a protein of interest which is expressed from the nucleic acid in the cell. An alternate embodiment, where the method employs the RNA counterpart of SEQ ID NO: 1 (i.e. SEQ ID NO: 4) is also contemplated.

In an alternate embodiment, there is provided a method of increasing expression, synthesis, or production of a protein of interest in a cell comprising the steps of: administering a nucleic acid to the cell, where the nucleic acid comprises a promoter, SEQ ID NO:1 and a sequence encoding a protein of interest which is expressed from the nucleic acid in the cell, where the increase in expression, synthesis or production of a protein of interest is relative to a similar step of administering a nucleic acid in the absence of SEQ ID NO:1. An alternate embodiment, where the method employs the RNA counterpart of SEQ ID NO: 1 (i.e. SEQ ID NO: 4) is also contemplated. In some embodiments, the method may additionally comprise a step of preparing the nucleic acid, wherein the promoter is positioned upstream or 5′ to SEQ ID NO: 1 or 4 and the sequence encoding a protein of interest is positioned downstream or 3′ to SEQ ID NO: 1 or 4. In some other embodiments, the method may additionally comprise a step of infecting a cell or a patient in need thereof, with HSV1 virus, wherein the step of infecting the cell occurs before administering the nucleic acid to the cell, which results in increased protein production in the cell, which could potentially help in treating or ameliorating the medical condition, cellular defect of disease that is being targeted. Alternatively, as noted hereinbefore, a live or oncolytic viral vector can be used to eliminate the step of pre-infecting the cell or the patient to augment increased protein production.

In some embodiments, the method may additionally comprise inserting the nucleic acid in a gene delivery vehicle, wherein the step of inserting the nucleic acid occurs before infecting the cell with HSV1 virus and before administering the nucleic acid to the cell, wherein the nucleic acid is administered to the cell by allowing the gene delivery vehicle containing the nucleic acid to infect the cell. The gene delivery vehicle can be selected from any of the non-limiting examples provided hereinbefore. As discussed earlier, the administration of nucleic acid containing SEQ ID NO: 1 or 4, results in increased protein production, by several fold, compared to its production in the absence of SEQ ID NO: 1 or 4.

The method may comprise using any of the nucleic acids, vectors, gene delivery vehicles, compositions or kits recited hereinbefore, or could be used to target any of the cell lines recited hereinbefore.

In the embodiments recited above, the promoter may be positioned upstream or 5′ to SEQ ID NO: 1 (or SEQ ID NO: 4) and the sequence encoding a protein of interest is positioned downstream or 3′ to SEQ ID NO: 1 (or SEQ ID NO: 4). The nucleic acid may be inserted in HSV1 and as noted earlier the desired effect of enhanced protein expression is observed when the cells are infected with HSV1 virus. In some embodiments, the method can be performed using the nucleic acid that is in the form of a plasmid, or a vector. The vectors can be selected from but not limited to viral vectors, live-viral vectors, oncolytic viral vectors, attenuated viral vectors, recombinant vectors or amplicon vectors.

For all the above method of production embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Methods of Treating Medical Conditions, Cellular Defects or Diseases

In an embodiment of the invention there is provided a method of improving or treating a medical condition, cellular defect or disease in a subject. The method comprises administering a nucleic acid to the cells of the subject showing the medical condition, cellular defect or disease, where the nucleic acid comprises a promoter, SEQ ID NO:1 and a sequence encoding a protein of interest which is expressed from the nucleic acid in the cells. The protein of interest selected is capable of improving or treating the medical condition, cellular defect or disease in the subject, and therefore, the expression, synthesis, or production of the protein of interest in the cells of the subject improves or treats the medical condition, cellular defect or disease in the subject. An alternate method, where the nucleic acid comprises SEQ ID NO: 4 is also contemplated.

In the nucleic acid, the promoter is positioned upstream or 5′ to SEQ ID NO: 1 (or SEQ ID NO: 4) and the sequence encoding a protein of interest is positioned downstream or 3′ to SEQ ID NO: 1 (or SEQ ID NO: 4). The nucleic acid may be inserted in HSV1, and to achieve the desired result, the subject or subject's cells are infected with HSV1, so as to allow delivery of the nucleic acid. As suggested earlier, the nucleic acid can be inserted in the HSV1, where it's a live-viral vector, an oncolytic viral vector, or an attenuated viral vector. An alternate embodiment, where the method employs the RNA counterpart of SEQ ID NO: 1 (i.e. SEQ ID NO: 4) may be possible.

In an embodiment of the invention, there is provided a method of treating a medical condition, cellular defect or disease in a patient comprising: administering a nucleic acid to the cells of the patient in need thereof, the nucleic acid comprising a promoter, SEQ ID NO:1 or 4, and a gene encoding a protein of interest which is expressed from the nucleic acid in the cells, wherein the protein of interest is capable of treating/ameliorating the medical condition; wherein the expression, synthesis, or production of the protein of interest in the cells of the patient ameliorates the medical condition, cellular defect or disease in the patient. More specifically, the transcription of SEQ ID NO: 1 or 4, increases translation of the gene encoding protein of interest by several fold, compared to its translation in the absence of SEQID NO: 1 or 4, and the large quantities of the protein of interest provide the necessary treatment. The method may additionally comprise the step of infecting the cell or the patient with HSV1 virus, wherein the step of infecting the cell or patient occurs before administering the nucleic acid to the cells. Additionally, the method may comprise the step of inserting the nucleic acid in any of the vectors, gene delivery vehicles, constructs, compositions or kits recited hereinbefore, wherein the step of inserting the nucleic acid occurs before infecting the cells or patient with HSV1 virus and before administering the nucleic acid to the cells or patient in need thereof. In the presence of HSV1 infection, the sequence SEQ ID NO: 1 or 4, increases protein expression by several fold (e.g. 8-fold).

In an alternate embodiment, a method of treating cancer in a subject is also provided. The method comprises administering a nucleic acid to the cells of the patient/subject, where the nucleic acid comprises a promoter, SEQ ID NO: 1 and a sequence encoding a protein of interest which is expressed from the nucleic acid in the cells. The protein of interest is selected carefully which is capable of ameliorating or treating cancer in the subject. Accordingly enhanced, expression, synthesis, or production of the protein of interest in the cells of the patient treats the cancer or carcinoma condition. In an alternate embodiment, the cancer could be melanoma. An alternate embodiment, where the method employs the RNA counterpart of SEQ ID NO: 1 (i.e. SEQ ID NO: 4) is also contemplated.

In an alternate embodiment, a method of treating melanoma in a subject is also provided. The method comprises administering a nucleic acid to the cells of the patient/subject, where the nucleic acid comprises a promoter, SEQ ID NO: 1 and a sequence encoding GM-CSF which is expressed from the nucleic acid in the cells. Accordingly, enhanced expression, synthesis, or production of GM-CSF in the cells of the patient treats the melanoma condition. An alternate embodiment, where the method employs the RNA counterpart of SEQ ID NO: 1 (i.e. SEQ ID NO: 4) is also contemplated.

The above method can be used to treat a variety of medical conditions, cellular defects or diseases. The following is a non-limiting list of examples carcinoma, melanoma, immunodeficiency, celiac disease, liver or kidney disorders, or any protein deficiency disorders.

For all the above method of treatment embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Methods of Improving Existing Gene Therapies

In an alternate embodiment there is provided a method of improving the efficacy of an existing gene therapy, where the method comprises modifying a gene delivery vehicle being used in the existing gene therapy by inserting a sequence of SEQ ID NO: 1, wherein the gene delivery vehicle comprises a sequence encoding a protein of interest. The modified gene delivery vehicle can be administered to the patient in need of the gene therapy. The transcription of SEQ ID NO: 1 increases translation of the sequence encoding the protein of interest, and thereby causing an increase in expression, production or synthesis of the protein of interest relative to its expression, production or synthesis in the absence of SEQ ID NO: 1, which improves the efficacy of the existing gene therapy. Accordingly, this method can be employed to improve the efficiency of any existing viral or non-viral based gene therapies. As exhibited in the experiments discussed above, the method can increase expression, production or synthesis of the protein of interest by several fold (e.g. 8-fold) compared to its expression, production or synthesis in the absence of SEQ ID NO: 1 or 4, thereby increasing the efficiency of the existing gene therapy. An alternate method, where the nucleic acid comprises the RNA counterpart of SEQ ID NO: 1 (i.e. SEQ ID NO: 4) is also contemplated. In some embodiments, the increase in expression, production or synthesis of the protein of interest is 0.5 fold, 1 fold, 2 fold, 3 fold, 4 fold, 5 fold, 6 fold, 7 fold, 8 fold or more compare to its expression, production or synthesis in the absence of SEQ ID NO: 1 (or SEQ ID NO: 4). The following is a non-limiting list of gene therapies for which the method can be used to increase efficiency: Zolgensma, Yescarta, Luxturna, Kymriah, Zynteglo, MB-107, Strimvelis, Tecartus or any of the existing gene therapies undergoing phase trials. Some examples of commercially available vectors that can be modified include T-vec (Amgen), HSV-1716 (Virttu Therapeutics-acquired by Sorrento), Immvira, Virogin, Replimune, Treovir, J&J, BeneVir, and Oncorus. To augment increased protein expression in non-HSV1 based gene therapies, coinfection with HSV1 virus or HSV1 live viral vector might be required. In an embodiment of the invention, the existing gene therapy can be an oncolytic viral therapy or a gene based immunotherapy or any other existing gene therapies].

For all the above method embodiments reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Method of Increasing Transgene Expression

A method of increasing transgene expression in a cell is provided, which comprises administering a nucleic acid to the cell, the nucleic acid comprising a promoter, SEQ ID NO: 1 and a transgene which is expressed from the nucleic acid in the cell. The transgene encodes a protein of interest, and the increase in expression of the transgene is relative to a similar step of administering a nucleic acid in the absence of SEQ ID NO: 1. The method can also be performed using the RNA counterpart SEQ D NO: 1 i.e. SEQ ID NO: 4.

For the above embodiment reciting SEQ ID NO: 1 or 4, alternate embodiments comprising any of the UL27 sequences i.e. SEQ ID NO: 7 or 10, and alternate embodiments comprising any of the UL19 sequences i.e. SEQ ID NO: 13 of 16, are also contemplated in the present invention.

### Method of Identifying a Transcription Start Site

In an embodiment of the invention, there is provided a method of identifying a transcription start site (TSS) (or 5′ UTR sequence or a leader sequence) in a viral genome that is capable of increasing protein expression, synthesis or production comprising: sequencing a copy of the viral genome to obtain sequencing data; mapping the sequencing data with pre-existing/annotated sequencing data of the viral genome by aligning short reads to identify splice junctions on mRNA transcripts of the viral genome; identifying a plurality of TSS by locating stacked short reads on each of the mRNA transcripts; carrying out reporter assays with a reporter protein using the identified TSS; and identifying the TSS that increases expression, synthesis or production of the reporter protein compared to a control assay in the absence of TSS. The multiple copies of the identified TSS for carrying out reporter assays may be obtained by amplification from a cDNA library and a copy of the viral genome may be obtained from virus infected cells. The mapping step may be carried out using RNA sequencing, or any of the advanced sequencing techniques available or being used currently. The reporter protein may be selected from any of following non-limiting examples: GFP, RFP, lacZ, LUC, CAT or any other reporter protein known in the art. The reporter assays may be conducted using reporter constructs containing the reporter protein and one of identified TSS, wherein the reporter constructs may be in the form of a plasmid, a vector, any gene delivery vehicle, or nanoparticles. In some embodiments, the TSS may be identified by locating the 5′ and 3′ ends of pre-existing/annotated sequencing data of the viral genome. To be certain, the reporter assays may be carried out in a plurality of cell lines or using a specific cell line selected from the non-limiting examples provided hereinbefore.

In some embodiments described above, the disclosure provides theory and speculation on the mechanism of biological processes. The present invention is not meant to be bound by theory or speculation as to the mechanisms involved in biological processes and the same should not be used to limit the invention in any way.

For all the above embodiments reciting US11, alternate embodiments with UL27 or UL19 5′ leaders are also contemplated.

### Discussion, Experimental Results and Examples

Determining HSV1 mRNA Sequences or 5′ Leader Sequence

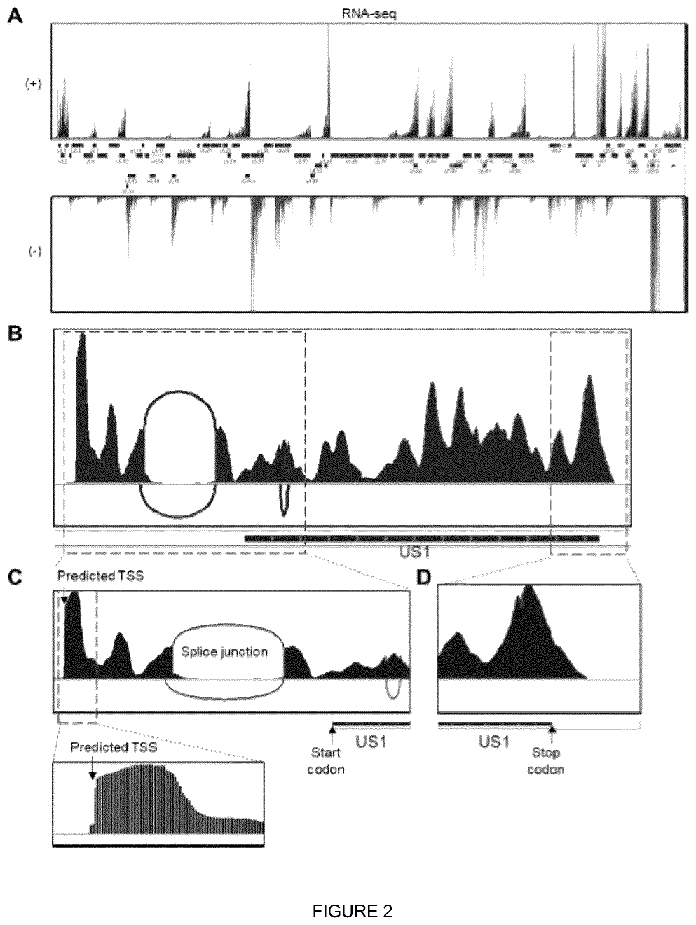

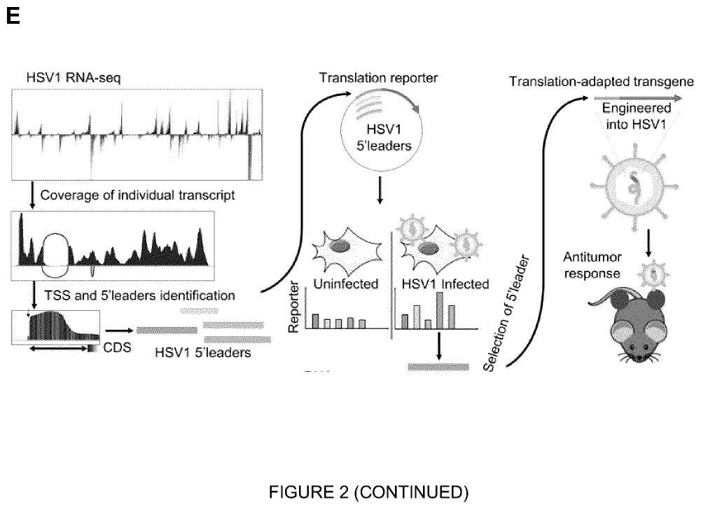

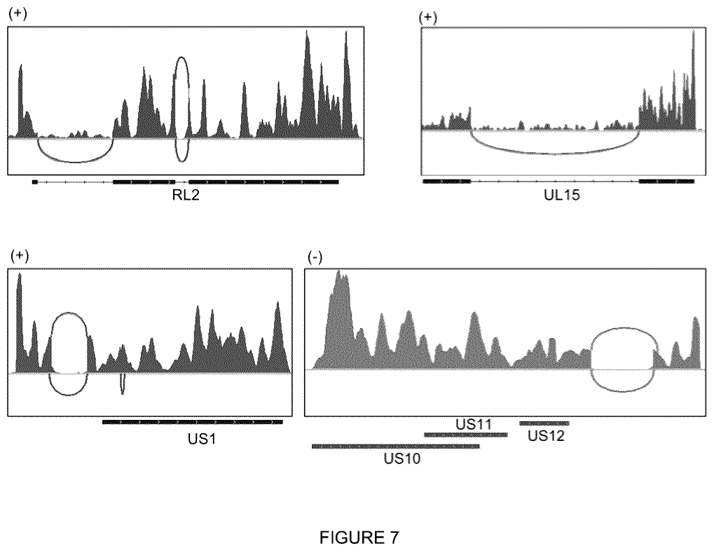

HSV1 is an enveloped dsDNA virus with a 153 Kb genome arranged in covalently linked long (L) and short(S) segments that collectively encode approximately 80 genes. Several single-gene studies have identified and characterized the 5′ leaders and 3′ untranslated regions (3′ UTRs) of a limited number of HSV1 genes (Table 1). Most transcripts, however, have no such annotations in the public NCBI database (e.g., NCBI accession number JQ780693 for strain KOS and JN555585 for strain 17). Therefore, RNA-Seq data of HSV1 infected 4T1 murine breast cancer cells was generated. From mapping the HSV1 reads from this dataset to the KOS strain reference (JQ780693.1), RNA transcripts originating from each of the positive and negative gDNA strands were distinguished (as shown in FIGS. 2A and 2E). FIG. 2A shows the total RNA-seq coverage of the HSV1 genome from 4T1 infected cells. It is pertinent to note that strand-specific RNA reads were mapped to HSV1 genome and separated by strand direction to avoid ambiguous mapping of overlapping genes. By mapping the HSV1 reads from this dataset to the KOS strain reference genome (JQ780693.1), one can distinguish RNA transcripts originating from each of the positive and negative gDNA strands (FIG. 2B). Additionally, the splice junctions of the four known spliced transcripts of HSV1 (i.e., UL15, US1, US12 and RL2) were also discerned in the process and the intron retention that was previously reported for RL2 (FIG. 7). As shown in FIG. 7, individual transcripts (RL2, UL15, US1, US12) are identified by RNA-seq coverage on the positive strand (blue) and negative strand (red).

It may be argued that identification of transcription start sites (TSS) from long-read sequencing techniques (e.g., PacBio methods) or full transcript sequencing (e.g., Oxford Nanopore MinION platform) can be more straightforward than RNA-Seq that relies on aligning short reads, especially when there are overlapping ORFs present. However, it was observed that standard RNA-Seq read mapping can sufficiently identify the TSS for non-overlapping HSV1 genes. This is made possible by the high depth and coverage achieved in infected cells, despite a low multiplicity of infection (MOI) of 0.1. The TSS locations were identified by monitoring for a “wall” of stacked short reads that were interpreted as the start of a transcript (as depicted in FIG. 2B and FIG. 2C). FIG. 2B shows RNA-seq coverage of the US1 gene from the HSV1 genome where intron-spanning reads were also detected and shown using Sashimi plot. FIG. 2C shows RNA-seq coverage in the 5′ region of US1 gene where the lower plot shows the region of the predicted TSS at nucleotide resolution. A similar approach was recently used by Whisnant et al. to enumerate the HSV1 TSS, although their methods employed more specialized RNA-Seq methods.

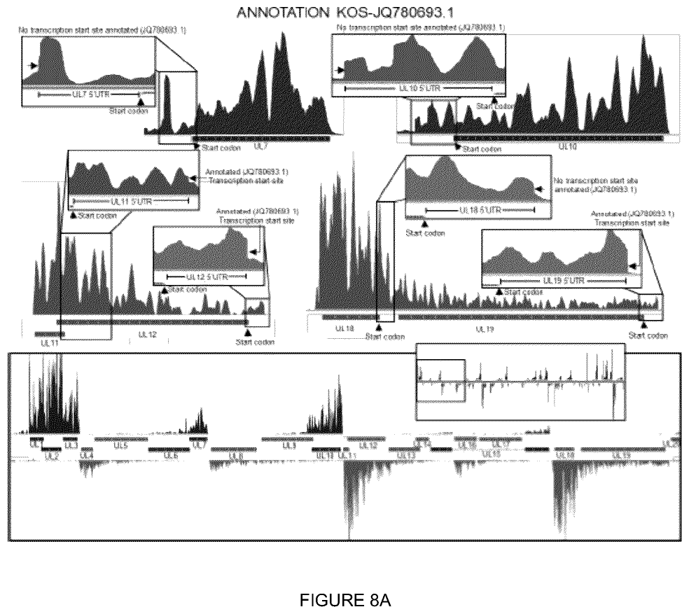

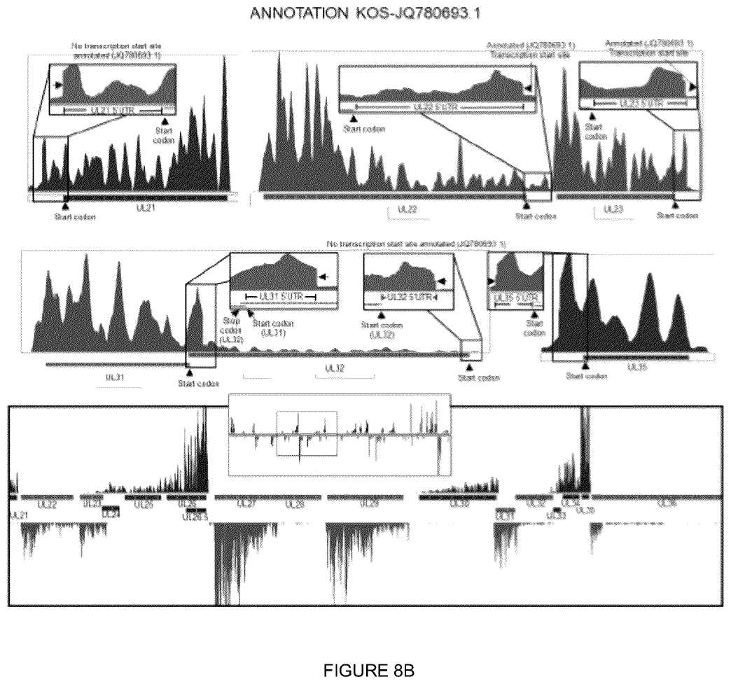

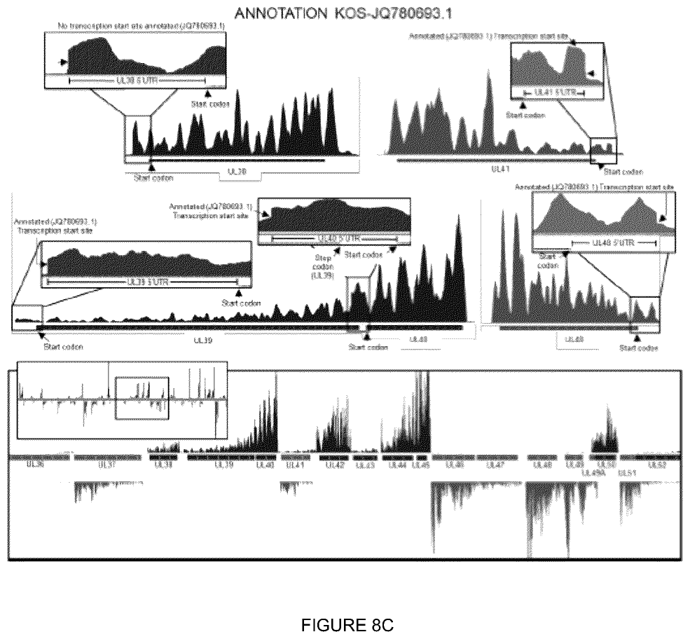

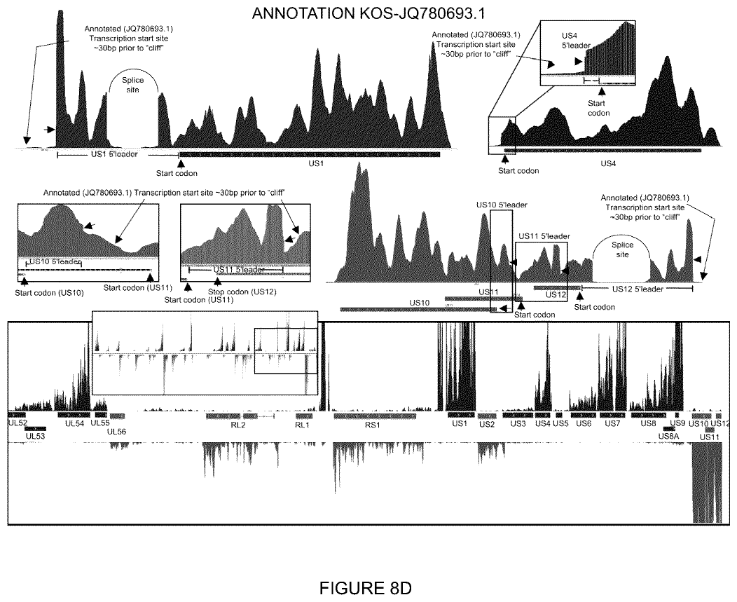

Through this experiment reads that flanked the 5′ and 3′ ends of most annotated ORFs were detected as shown in FIGS. 2C and 2D, confirming that all annotated HSV1 transcripts harbor both a 5′ leader and a 3′ UTR. The RNA-seq coverage at the 3′ region of US1 gene can be clearly seen in FIG. 2D. Although the 3′ UTRs of a large portion of the viral genes overlap with a downstream ORF, a spike in read density at a single nucleotide position upstream of the start codon consistent with a TSS could be clearly distinguished from low read density across 3′ UTRs (FIG. 2B, 2C, insets and Table 1). Schematic overview of the workflow to identify HSV1 5′leaders from RNA-seq read, screen for translation enhancing 5′leaders specifically in HSV1-infected cells, and incorporate the 5′leader into transgene expression in an oncolytic HSV1 genome to test in a tumor model in vivo can be seen in FIG. 2E. Using these TSS coordinates, we identified 61 5′leader sequences of HSV1 genes (Table 2). Importantly, when comparing the identified TSSs with the few annotated in NCBI, more recently identified by long-read sequencing done by Tombacz et al., or RNA-Seq on enriched 5′ end reads by Whisnant et al., we found that the coordinates of the TSSs were exact or differed by only a few nucleotides (Table 1). The read density coverage at each identified TSS are more clearly shown in FIG. 8. Total RNA-seq coverage of the HSV1 genome is shown in FIG. 8, and RNA-seq data from 4T1 infected with HSV1 that was previously published in Hoang et al., 2019. To avoid ambiguous mapping of overlapping genes, strand-specific RNA reads were mapped to HSV1 (JQ780693.1) and separated by direction. The graph shown in FIG. 8 shows the inset for every identified TSSs of all HSV1 genes.

Using these TSS coordinates, the inventors were able to identify 61 5′leader sequences of HSV1 genes (see table 2). Importantly, when comparing the identified TSSs with the few annotated in NCBI, more recently identified by long-read sequencing done by Tombácz et al, or RNA-Seq on enriched 5′ end reads by Whisnant et al, the inventors found that the coordinates of the TSSs were exact or differed by only a few nucleotides (Table 1). These studies confirmed that the approach was robust to detect and confidently annotate each HSV1 transcript 5′ leader.

TABLE 1
HSV1 TSS identified in this study (Hoang et al.) compared to Tombacz et al., 2017 and Whisnant et al., 2020
HSV1 TSS identified in this study (Hoang et al) compared with Tombacz et al [symbol] and Whisnant et al

[symbol]

| | Hoang et al (This study) | | | | Tomb\dot{a}cz et al | | Whisnant et al | |
|---|---|---|---|---|---|---|---|---|
| | TSS | 5′UTR | | TSS | TSS | Differences | TSS | Differences |
| HSV1 | coordinates | coordinates | 5′ UTA | coordinates | coordinates | to our | coordinates | to our |
| Genes | (JQ780693.1) | (J [symbol] 780693.1) | length | (JN555585) | (JN555585) | study (nt) | (JN555585) | study (nt) |
| UL1+ | 8901 | JQ780693.1 [symbol] :8901-8994 | 92 | 9245 | 9242 | 3 | 9245 | 0 |
| UL2+ | 9396 | JQ780693.1 [symbol] :9396-9541 | 145 | 9740 | 9739 | 1 | 9739 | 1 |
| UL3+ | X | | | | 10736 | | | |
| UL4− | 12 151 | JQ780693.1−:12079-12151 | 72 | 12495 | 12494 | 1 | 12494 | 1 |
| UL5− | X | | | | 15581 | | | |
| UL6+ | X | | | | 15032 | | | |
| UL7+ | 16 613 | JQ780693.1 [symbol] :16613-16792 | 179 | 16957 | 16956 | 1 | 16956 | 1 |
| UL8− | 20 337 | JQ780693.1−:20134-20337 | 203 | 20680 | 20677 | 3 | 20679 | 1 |
| UL9− | X | | | | 23358 | | | |
| UL10+ | 22 601 | JQ780693.1 [symbol] :22601-22862 | 261 | 22944 | 22943 | 1 | 22943 | 1 |
| UL11− | 25 148 | JQ780693.1−:24741-25148 | 407 | 25499 | 25498 | 1 | 25498 | 1 |
| UL12− | 26 695 | JQ780693.1−:26537-26695 | 158 | 27046 | 27045 | 1 | 27045 | 1 |
| UL13− | X | | | | 28657 | | | |
| UL14− | X | | | | 29249 | | | |
| UL15+ | 28 451 | JQ780693.1 [symbol] :28451-28670 | 219 | 28802 | 28801 | 1 | 28801 | 1 |
| UL16− | 31 261 | JQ780693.1−:30949-31261 | 312 | 31608 | 31607 | 1 | 31607 | |
| UL17− | X | | | | 33719 | | | |
| UL18− | 35 902 | JQ780693.1−:35704-35902 | 198 | 36250 | 36249 | 1 | 36247 | 3 |
| UL19− | 40 421 | JQ780693.1−:40182-40421 | 239 | 40768 | 40768 | 0 | 40766 | 2 |
| UL20− | X | | | | 41613 | | | |
| UL21+ | 41 515 | JQ780693.1 [symbol] :41515-41726 | 211 | 41863 | 41862 | 1 | 41862 | 1 |
| UL22− | 46 230 | JQ780693.1−:46031-46230 | 199 | 46582 | 46581 | 1 | 46581 | 1 |
| UL23− | 47 558 | JQ780693.1−:47451-47558 | 107 | 47910 | 47906 | 4 | 47909 | 1 |
| UL24+ | 47 008 | JQ780693.1 [symbol] :47008-47386 | 378 | 47360 | 47671 | 311 | 47407 | 47 |
| UL25+ | 47 722 | JQ780693.1 [symbol] :47722-48461 | 739 | 48074 | 48630 | 556 | 48630 | 556 |
| UL26+ | 50 311 | JQ780693.1 [symbol] :50311-50469 | 158 | 50664 | 50659 | 5 | 50663 | 1 |
| UL26.5+ | 51 249 | JQ780693.1 [symbol] :51294-51387 | 93 | 51635 | 51636 | 1 | 51634 | 1 |
| UL27− | 55 744 | JQ780693.1−:55458-55744 | 286 | 56081 | 56080 | 1 | 56080 | 1 |
| UL29− | 61 979 | JQ780693.1−:61717-61979 | 262 | 62312 | 62312 | 0 | 62313 | 1 |
| UL30+ | X | | | | 62605 | | | |
| UL31− | 67 123 | JQ780693.1−:67044-67123 | 79 | 67458 | 67458 | 0 | 67457 | 1 |
| UL32− | 68 885 | JQ780693.1−:68827-68885 | 58 | 69220 | 69220 | 0 | 69219 | 1 |
| UL33+ | 68 730 | JQ780693.1 [symbol] :68730-68826 | 96 | 69065 | 69065 | 0 | 69064 | 1 |
| UL34+ | 69 107 | JQ780693.1 [symbol] :69107-69298 | 191 | 69442 | 69442 | 0 | 69441 | 1 |
| UL35+ | 70 151 | JQ780693.1 [symbol] :70151-70231 | 80 | 70486 | 70489 | 3 | 70488 | 2 |
| UL36− | X | | | | ? | | | |
| UL37− | 83 780 | JQ780693.1−:83649-83780 | 131 | 84215 | 84215 | 0 | 84214 | 1 |
| UL38+ | 83 958 | JQ780693.1 [symbol] :83958-84088 | 130 | 84393 | 84394 | 1 | 84393 | 0 |
| UL39+ | 85 774 | JQ780693.1 [symbol] :85774-86000 | 226 | 86217 | 86214 | 3 | 86216 | 1 |
| UL40+ | 89 331 | JQ780693.1 [symbol] :89331-89482 | 151 | 89774 | 89775 | 1 | 89773 | 1 |
| UL41− | 92 312 | JQ780693.1−:92193-92312 | 119 | 92755 | 92755 | 0 | 92753 | 2 |
| UL42+ | 924 191 | JQ780693.1 [symbol] :92491-92669 | 178 | 92934 | 92936 | 2 | 92934 | 0 |
| UL43+ | 94 323 | JQ780693.1 [symbol] :94323-94357 | 34 | 94765 | 94653 | 112 | 94764 | 1 |
| UL44+ | 95 723 | JQ780693.1 [symbol] :95723-95865 | 142 | 96171 | 96174 | 3 | 96173 | 2 |
| UL45+ | 95 706 | JQ780693.1 [symbol] :95706-97585 | 79 | 96154 | 97953 | −1799 | 97953 | −1799 |
| UL46− | X | | | | 100995 | | | 0 |
| UL47− | 102 866 | JQ780693.1−:102672-102866 | 194 | 103312 | 103311 | 1 | 103309 | 3 |
| UL48− | 104 814 | JQ780693.1−:104635-104814 | 179 | 105258 | 105257 | 1 | 105257 | 1 |
| UL49− | 106 099 | JQ780693.1−:105949-106099 | 150 | 106542 | 106543 | −1 | 106538 | 4 |
| UL49A− | 106 686 | JQ780693.1 [symbol] :106551-106686 | 135 | 107129 | 107128 | 1 | 107126 | 3 |
| UL50+ | 106 387 | JQ780693.1 [symbol] :106387-106568 | 181 | 106830 | 106826 | 4 | 106829 | 1 |
| UL51− | 108863 | JQ780693.1−:108569-108863 | 294 | 109306 | 109305 | 1 | 10930 [symbol] | |
| UL52+ | X | | | | 108985 | | | |
| UL53+ | 111501 | JQ780693.1 [symbol] :111501-111737 | 236 | 111944 | 111943 | 1 | 111943 | 1 |
| UL54+ | 113153 | JQ7806931 [symbol] :113153-113292 | 139 | 113596 | 113598 | −2 | 113598 | 2 |
| UL55+ | 115002 | JQ7806931 [symbol] :115002-115054 | 52 | 115445 | 115447 | −2 | 115444 | 1 |
| UL56− | 116643 | JQ7806931 [symbol] :116485-116643 | 158 | 117084 | 117083 | 1 | 117083 | 1 |
| RL2− | 123628 | JQ780693.1−:123482-123628 | 146 | 124258 | 124256 | 2 | 124255 | 3 |
| RL1− | 125310 | JQ7806931 [symbol] :125206-125310 | 104 | 125965 | 125944 | 21 | 125964 | 1 |
| RS1− | 130404 | JQ780693.1−:130103-130404 | 301 | 131431 | 131428 | 3 | 131429 | 2 |
| US1+ | 131098 | | 602 | | | | | 0 |
| | | JQ780693.1+:131098-131700 | 354 (spliced) | 132128 | 132127 | 1 | 132129 | −1 |
| US2− | 134359 | JQ780693.1−:133983-134359 | 376 | 135307 | 135306 | 1 | 135305 | 2 |
| US3+ | 134019 | JQ780693.1+:134019-134277 | 258 | 134967 | 134958 | 9 | 134966 | 1 |
| US4+ | 135787 | JQ780693.1+:135787-135799 | 12 | 136735 | 136733 | 2 | 136734 | 1 |
| US5+ | X | | | | 137625 | | | 0 |
| US6+ | 137395 | JQ780693.1+:137395-137476 | 81 | 138342 | 138338 | 4 | 138345 | −3 |
| US7+ | 138754 | JQ780693.1+:138754-138843 | 89 | 139699 | 139698 | 1 | 139699 | 0 |
| US8+ | 140208 | JQ780693.1+:140208-140282 | 74 | 141173 | 141170 | 3 | 141171 | 2 |
| US8A+ | 141665 | JQ780693.1+:141665-141783 | 118 | 142630 | 142626 | 4 | 142629 | 1 |
| US9+ | 142287 | JQ780693.1+:142287-142352 | 65 | 143252 | 143250 | 2 | 143251 | 1 |
| US10− | 144183 | JQ780693.1−:144114-144183 | 69 | 145169 | 145171 | −2 | 145168 | 1 |
| US11− | 144480 | JQ780693.1−:144265-144480 | 215 | 145466 | 145461 | 5 | 145461 | 5 |
| US12− | 145247 | JQ780693.1−:144596-145247 | 319 (spliced) | 146069 | 146068 | 1 | 146066 | 3 |

[symbol]

indicates data missing or illegible when filed

TABLE 2
Sequence of HSV1 leaders identified in this study (Hoang et al.).
Sequence of HSV1 5′leaders identified in this study (Hoang et af)

| Gene | | |
|---|---|---|
| name | 5′leader | |
| (strand) | length | 5′leader sequence |
| UL1+ | 92 | [symbol] |
| UL2+ | 145 | [symbol] |
| | | [symbol] |
| UL3+ | 221 | [symbol] |
| | | [symbol] |
| | | [symbol] |
| UL4− | 72 | [symbol] |
| UL5− | 450 | [symbol] |
| | | [symbol] |
| | | [symbol] |
| | | [symbol] |
| | | [symbol] |
| UL6+ | 98 | [symbol] |
| UL7+ | 179 | [symbol] |
| | | [symbol] |
| UL8− | 203 | [symbol] |
| | | [symbol] |
| UL9− | 99 | [symbol] |
| UL10+ | 261 | [symbol] |
| | | [symbol] |
| | | [symbol] |
| UL11− | 407 | [symbol] |
| | | [symbol] |
| | | [symbol] |
| | | [symbol] |
| | | [symbol] |
| UL12− | 158 | [symbol] |
| | | [symbol] |
| UL13− | 155 | [symbol] |
| | | [symbol] |
| UL14− | 334 | [symbol] |
| | | [symbol] |
| | | [symbol] |
| | | [symbol] |
| UL15+ | 219 | [symbol] |
| | | [symbol] |
| | | [symbol] |
| UL16- | 312 | [symbol] |
| | | [symbol] |
| | | [symbol] |
| | | [symbol] |
| UL17- | 222 | [symbol] |
| | | [symbol] |
| | | [symbol] |
| UL18- | 198 | [symbol] |
| | | [symbol] |
| | | [symbol] |
| UL19- | 239 | [symbol] |
| | | [symbol] |
| | | [symbol] |
| UL20- | 125 | [symbol] |
| | | [symbol] |
| UL21+ | 211 | [symbol] |
| | | [symbol] |
| | | [symbol] |
| UL22- | 199 | [symbol] |
| | | [symbol] |
| | | [symbol] |
| UL23- | 107 | [symbol] |
| | | [symbol] |
| UL24+ | 378 | [symbol] |
| | | [symbol] |
| | | [symbol] |
| | | [symbol] |
| | | [symbol] |
| UL25+ | 739 | [symbol] |
| | | [symbol] |
| | | [symbol] |
| | | [symbol] |
| | | [symbol] |
| | | [symbol] |
| | | [symbol] |
| | | [symbol] |
| | | [symbol] |
| UL26+ | 158 | [symbol] |
| | | [symbol] |
| U126.5+ | 93 | [symbol] |
| UL27- | 286 | [symbol] |
| | | [symbol] |
| | | [symbol] |
| | | [symbol] |
| UL28- | 270 | [symbol] |
| | | [symbol] |
| | | [symbol] |
| | | [symbol] |
| UL29+ | 262 | [symbol] |
| | | [symbol] |
| | | [symbol] |
| UL30+ | 202 | [symbol] |
| | | [symbol] |
| | | [symbol] |
| UL31- | 79 | [symbol] |
| UL32- | 58 | [symbol] |
| UL33+ | 96 | [symbol] |
| | | [symbol] |
| UL34+ | 191 | [symbol] |
| | | [symbol] |
| UL35+ | 80 | [symbol] |
| UL36- | 75 | [symbol] |
| UL37- | 131 | [symbol] |
| | | [symbol] |
| UL38+ | 130 | [symbol] |
| | | [symbol] |
| UL39+ | 226 | [symbol] |
| | | [symbol] |
| | | [symbol] |
| UL40+ | 151 | [symbol] |
| | | [symbol] |
| UL41- | 119 | [symbol] |
| | | [symbol] |
| UL42+ | 178 | [symbol] |
| | | [symbol] |
| UL43+ | 34 | [symbol] |
| UL44+ | 142 | [symbol] |
| | | [symbol] |
| UL45+ | 79 | [symbol] |
| UL46- | 43 | [symbol] |
| UL47- | 194 | [symbol] |
| | | [symbol] |
| | | [symbol] |
| UL48- | 179 | [symbol] |
| | | [symbol] |
| UL49- | 150 | [symbol] |
| | | [symbol] |
| UL49A- | 135 | [symbol] |
| | | [symbol] |
| UL50+ | 181 | [symbol] |
| | | [symbol] |
| UL51- | 294 | [symbol] |
| | | [symbol] |
| | | [symbol] |
| | | [symbol] |
| UL52+ | 63 | [symbol] |
| UL53+ | 236 | [symbol] |
| | | [symbol] |
| | | [symbol] |
| UL54+ | 139 | ggtggtgtgcagccgtgttccaaccacggtcacgcttcggtgcctctccccgattcgggcccggtcgctcgctaccggtg |
| | | cgccaccaccagaggccatatccgacaccccagccccgacggcagccgacagcccggtc-atg |
| UL55+ | 52 | gtcatagtgcccttaggagcttcccgcccgggcgcatccccccttttgcact-atg |
| UL56- | 158 | ggttgggcgacgcatgccagcccaacaaaatccgccggggtgccagtcccattcccgaaggcgtagcccgttaacttggc |
| | | tggcttggatggggagtagggccttttccattaccccaaggacctagcgcgcgggagtcgtggctttggggcgcatcc- |
| | | atg |
| RL2- | 146 | tcgcatttgcacctcggcactcggagcgagacgcagcagccaggcagactcgggccgccccctctccgcatcaccacag |
| | | aagccccgcctacgttgcgacccccagggaccctccgtccgcgaccctccagccgcatacgaccccc-atg |
| RL1- | 104 | cccccgcggccgagactagcgagttagacaggcaagcactactcgcctctgcacgcacatgcttg |
| | | cctgtcaaactctaccaccccggcacgctctctgtctcc-atg |
| RS1- | 301 | ccacacggagcgcggctgccgacacggatccacgacccgacgcgggaccgccagagacagaccgtcagacgctcgccg |
| | | cgccgggacgccgatacgcggacgaagcgcgggagggggatcggccgtccctgtcctttttcccacccaagcatcgaccgg |
| | | tccgcgctagttccgcgtcgacggcgggggtcgtcggggtccgtgggtctcgccccctccccccatcgagagtccgtaggt |
| | | gacctaccgtgctacgtccgccgtcgcagccgtatccccggaggatcgccccgcateggcg-atg |
| US1+ | 517 | gcagacggcgccggccacgaacgacgggagcggctgcggagcacgcggaccgggagcgggagtcgcagagggccg |
| | | tcggagcggacggcgtcggcatcgcgacgccccggctcgggatcgggatcgcatcggaaagggacacgcggacgcggg |
| | | ggggaaagacccgcccaccccacccacgaaacacaggggacgcaccccgggggcctccgacgacagaaacccaccgg |
| | | tccgccttttttgcacgggtaagcaccttggggggcggaggagggggggacgcgggggcggaggaggggggacgcggggg |
| | | cggaggaggggggacgcgggggcggaggaggggggacgcgggggcggaggaggggggacgcgggggcggaggaggggg |
| | | ctcacccgcgttcgtgccttcccgcaggaggaacgtcctcgtcgaggcgaccggcggcgaccgttgcgtggaccgcttcct |
| | | gctcgtcgggcggggggaagccactgtggtcctccgggacgttttctgg-atg |
| US2- | 376 | gacggctttgtctccggcgggacggcctcctccttcctcctgccctgtcccccgtaaacgcgacaaaacttacgacaggcc |
| | | attcgccgcaccgtgagtgccaaccaacgagcaccccgaacgacgggccccggggttttaaggagcggcagtttgacgacc |
| | | caccccctgacctacccccccgtaaatcaccctcccctcccccggacgcctccgctgccggtcgctccaagggcccccccg |
| | | ggaaggcgggtctgtggaccgtagggcccttaaatttttagagcagcccccgcgtcggcctgtctccccgccgtgcgtggc |
| | | cttacaaatctgcaagtgccccaaatcggacacgggcctgtaatataccaac-atg |
| US3+ | 258 | acttgcagatttgtaaggccacgcacggggggagacaggccgacgcgggggctgctctaaaaatttaagggccctacgg |
| | | tccacagacccgccttcccgggggggcccttggagcgaccggcagcggaggcgtccgggggaggggagggtgatttac |
| | | gggggggtaggtcagggggtgggtcgtcaaactgccgctccttaaaaccccggggcccgtcgttcggggtgctc |
| | | gttggttggcactcacggtgcggcga-atg |
| US4+ | 12 | gtttttggcatc-atg |
| US5+ | 106 | gcgcgacttccgggcctcagaacccacccgaaacggccaacggacgtctgagccaggcctggctatcc |
| | | ggagaaacagcacacgacttggcgttctgtgtgtcgcg-atg |
| US6+ | 81 | ggtcataagcttcagcgcgaacgaccaactaccccgatcatcagttatccttaaggtctcttttgtgtggtgcgttccgg |
| | | t-atg |
| US7+ | 89 | gtggacagtcgataagtcggtagcgggggacgcgcacctgttccgcctgtcgcacccacagctttttttgegaaccgtccc |
| | | gttccggg-atg |
| US8+ | 74 | tcttctggcgggttggtgcggtgctgtttgttgggctcccattttacccgaagatcggctgctatccccgggac-atg |
| US8A+ | 118 | acgtacattcgcgtggccgacagcgagctgtacgcggactggagctcggacagcgagggagaacgcgaccagg |
| | | tcccgtggctggcccccccggagagacccgactctccctccacca-atg |
| US9+ | 65 | agcaaattaaaaatgtgagtcacagcgaccgcaacttcccacccggagctttcttccggcctcg-atg |
| US10- | 69 | accccagaggtgttcacgcacctcgaggacacccgcgcatgatctccggacccccgcaacggggtgata-atg |
| US11- | 215 | [symbol] |
| | | [symbol] |
| | | [symbol] |
| US12- | 486 | [symbol] |
| | | [symbol] |
| | | [symbol] |
| | | [symbol] |
| | | [symbol] |
| | | [symbol] |
| | | [symbol] |

[symbol]

indicates data missing or illegible when filed

### The US11 Leader Enhances Translation of Downstream ORFs in HSV1-Infected Cells

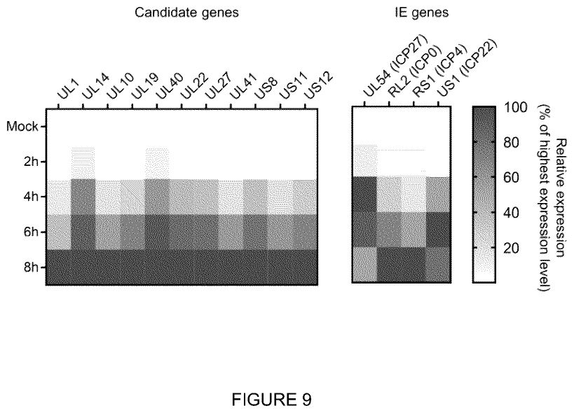

After identifying the 5′ leader sequences of most HSV1 genes in silico, it was important to determine their ability to modify the translation output of a downstream cistron. It was speculated that the leaders of HSV1 late genes that are expressed in an established infection stage should be best adapted to the altered translation control of HSV1 infection, and therefore should be more likely to possess motifs that positively modify translation in HSV1 infected cells. To confirm this hypothesis, ten late genes were selected for testing the translation modification effect of their 5′ leader in HSV1 infected cells (as shown in FIG. 9). Relative mRNA expression level of the 10 late genes selected as candidates is shown on the left heatmap, as well as relative mRNA expression level of the 4 immediate early genes is shown in the right heatmap. mRNA expression were obtained from a previously reported study by Rutkowski et al., 2015. To normalize the levels as percentages, expression level was normalized as a percentage of the highest expression level across all time points.

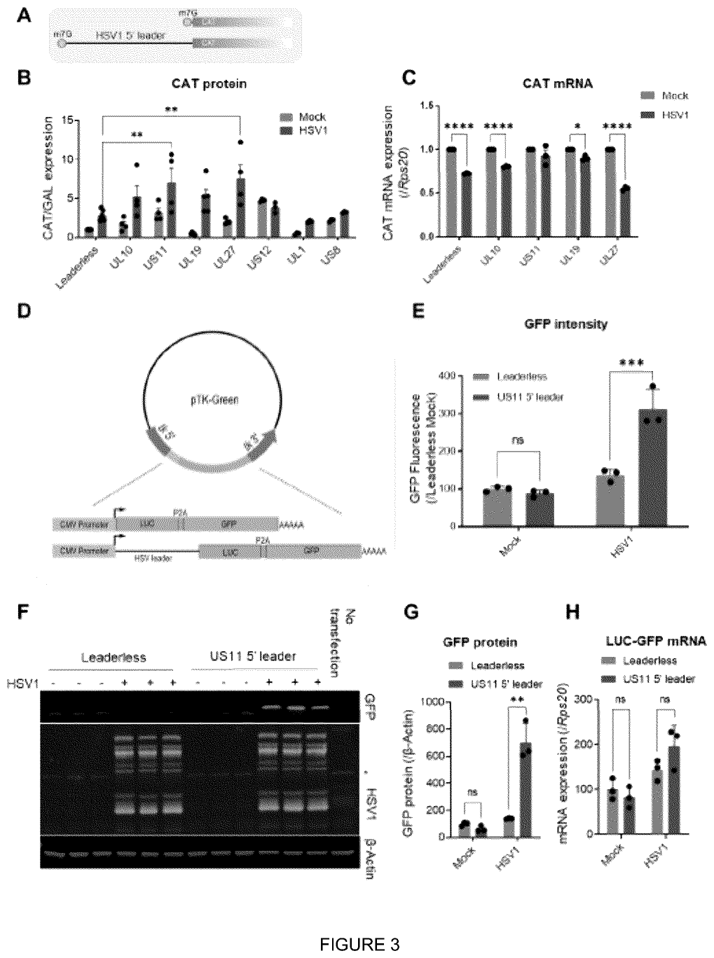

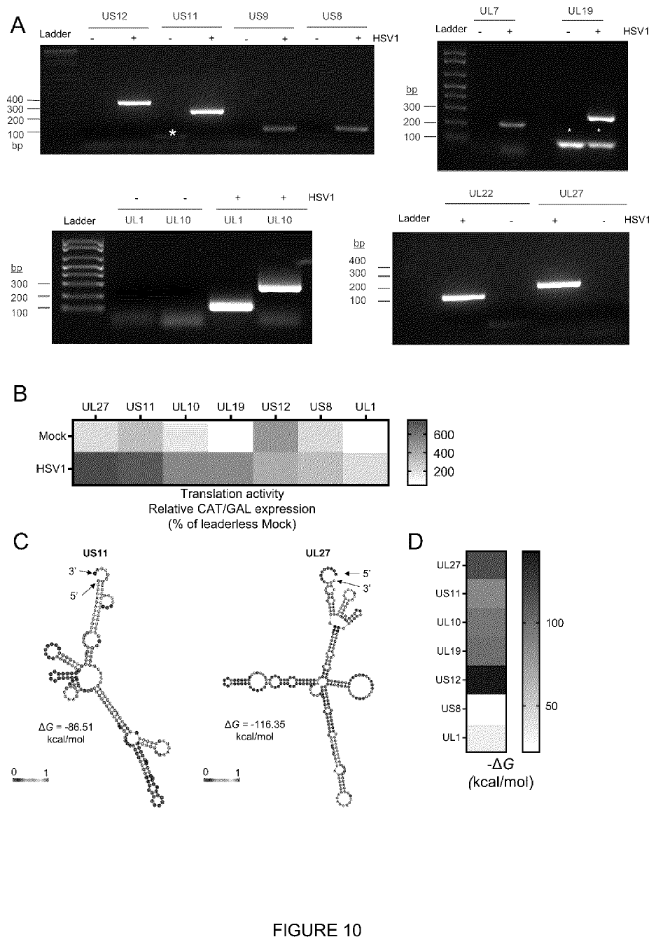

HSV1 5′ leaders of selected late genes were amplified from a cDNA library generated from HSV1-infected 4T1 cells and inserted upstream of the chloramphenicol acetyltransferase (CAT) reporter construct (FIG. 3A and FIG. 10A). FIG. 3 shows HSV1 US11 5′ leader sequences that enhance expression of protein reporters in HSV1-infected mammalian cells. Specifically, FIG. 3(A) provides schematic diagrams of the mRNAs expressed from CAT reporter construct with or without HSV1 5′UTR sequence. Furthermore, FIG. 10 provides a translation reporter screen for 5′UTRs that enhance translation during HSV1 infection and FIG. 10(A) provides agarose gel visualization of other HSV1 5′leaders (including the rest of the 5′ leaders shown in FIG. 3) amplified from total RNA of HSV1 infected cells.

Negative controls (i.e. RNA isolated from uninfected cells) confirmed the specificity of the PCRs products for HSV1 transcripts only. The translation reporter assay was also performed in uninfected and HSV1-infected conditions by co-transfecting 4T1 cells with monocistronic plasmids expressing CAT and β-galactosidase post-infection, wherein the latter construct was included to standardize differences in transfection efficiency. In uninfected cells, the viral 5′ leaders had both positive and negative effects on reporter expression, although no significant trends were observed (as shown in FIG. 3B). FIG. 3B shows results of the translation reporter assay to screen for HSV1 5′ leader sequences that enhance translation during HSV1 infection. Precisely, 4T1 cells were infected with HSV-1716-GFP at an MOI of 5, then transfected with the CAT plasmid and a β-GAL expression plasmid served as a transfection control. Cells were lysed 24 hours post infection and CAT expression was quantified by ELISA, while β-GAL activity was quantified by colorimetric assay using ONPG substrate. Two-way ANOVA with Tukey's post-hoc test was also performed. In the figure, only significant tests are shown, wherein n=at least 3 biological replicates and the error bars indicate standard deviation (sd). * p<0.05, ** p<0.01.

The US11 5′leader was highest in augmenting the translation of CAT mRNA reporter. In contrast, the 5′leaders of UL1 and US8 were found to have none to inhibitory effects during HSV1 infection (FIG. 10B). FIG. 10B shows translation reporter assay to screen for HSV-1 leader sequences that enhance translation during HSV-1 infection. 4T1 cells were transfected with the CAT plasmid and a β-GAL expression plasmid that serve as a transfection control. 8 hours post transfection, cells were infected with HSV-1716-GFP at an MOI of 5. Cells were lysed 18 hours post infection and CAT expression was quantified by ELISA, while β-GAL activity was quantified by colorimetric assay using ONPG substrate.

However, following HSV1 infection, we found that US11 and UL27 5′leaders significantly enhanced CAT protein expression (FIG. 3B) compared with the reporter lacking a leader (leaderless). Importantly, these observations are not through 5′leader-mediated upregulation of CAT mRNA transcription (FIG. 3C).

Following HSV1 infection, it was observed that that 5′UTR genes, specifically US11 and UL27 5′ leaders, significantly enhanced CAT expression (FIG. 3B) compared to the reporter construct lacking a leader (i.e. leaderless). Additionally, the UL19 5′leader enhanced CAT protein expression in the HSV1-infected condition, yet inhibited CAT protein expression in the uninfected condition. It is important to note that these observations are not through 5′ leader-mediated upregulation of CAT mRNA transcription (as shown in FIG. 3C). FIG. 3C shows relative CAT mRNA expression from CAT translation reporter assay, where 4T1 cells were treated as in 2 (C), then lysed using Trizol. RT-qPCR was then used to quantify mRNA expression of CAT mRNA normalized to the expression of Rps20. Additionally, two-way ANOVA with Sidak's post-hoc test was also performed. In the figure, only significant tests are shown, wherein n=3 biological replicates and error bars indicate standard deviation (sd). * p<0.05, ** p<0.01, **** p<0.0001.

It was also predicted that the folding free energy and potential secondary structure of the US11 and UL27 leaders (FIG. 10C) as well as the folding free energy of other screened HSV1 leaders (FIG. 10D). FIG. 10B shows the predicted secondary structure and folding free energy of US11 (left panel) and UL27 (right panel) leaders using Vienna RNAfold, where the color scale bars represent base pairing probabilities. FIG. 10(C) shows the heatmap representing folding free energy of candidate HSV1 leaders, calculated using Vienna RNAfold.

It was observed that despite having the strongest translation enhancement on CAT expression, the 5′ leaders of US11 and UL27 have low predicted folding free energy compared to other HSV1 leaders. Together, these results suggest that the 5′ leader sequences from US11 or UL27 mRNAs can mediate HSV1 infection-dependent increases in protein expression when inserted upstream of a transgene in cells.

It has been reported that lytic infection by HSV1 induces a profound reprogramming of cellular transcription, splicing and nuclear export. Thus, a plasmid-based overexpression reporter assay might be compromised by HSV1 infection and not faithfully reflect the gene expression processes, including mRNA translation, of HSV1-encoded transgenes. Therefore, the effect of US11 5′ leaders on regulating transgene expression directly from expression cassettes designed to be inserted within the tk locus of the HSV1 genome was investigated. Without limiting the scope of the present application, it is noted that transcription from the pTK plasmid expression cassette is driven by a CMV promoter and includes a SV40 polyadenylation signal (as shown in FIG. 3D). However, any other upstream promoters, enhancers, or regulatory elements could be used with the 5′ leader sequences. FIG. 3D provides a schematic diagram of the pTK-Green plasmid that harbors the HSV1 5′ leader-reporter construct for insertion into the HSV1 TK gene and the resulting transcripts. As seen in the figure, the ribosome skipping sequence P2A is inserted between the luciferase CDS and GFP CDS, which allows for synthesis of two proteins from one cistron. This bicistronic transgene cassette was created to allow concomitant expression of the therapeutic protein along with a reporter protein to facilitate recombinant virus selection and monitoring, all under the control of putative enhancer elements inserted at the 5′ end of the expression cassette (as can be seen in FIG. 3D). The ORFs consisted of luciferase (LUC; however, any protein of interest can be used and LUC can be replaced to create the desired therapeutic ORF). Additionally, the green fluorescence protein (GFP) separated by the self-cleaving peptide porcine teschovirus-1 2A (P2A) was inserted in the ORF. It is known that inclusion of P2A causes intercistronic translating ribosomes to skip peptide bond formation between the glycine and proline residues, resulting in production of separate LUC and GFP proteins from a single luc-gfp mRNA transcript. Simultaneously, GFP fluorescence was quantified in 4T1 cells transfected with the leaderless plasmid or a plasmid harboring the US11 leader and subsequently infected with or without HSV1 and GFP expression was monitored by fluorescence microscopy. As shown in FIG. 3E, low fluorescence was observed in leaderless and uninfected cells.

FIG. 3E shows quantification of GFP fluorescence in cells that were transfected with LUC-GFP reporter plasmid, then infected with the HSV1 (KOS strain referenced above) 4 hours post transfection at an MOI of 2.5 and images were taken at 24 hours post infection. As expected, the construct harboring the US11 leader showed significant GFP expression, however this spike in expression was observed only in cells infected with HSV1. A further western blot analysis of transfected cell lysates confirmed that inclusion of the US11 5′ leader confers an increase in GFP protein levels in HSV1-infected cells (as shown in FIGS. 3F and 3G). FIG. 3(F) shows western blot of lysate from 293T cells that were treated as described in FIG. 2(B) with antibodies against GFP, anti-HSV1 or anti-β-Actin antibodies. FIG. 3(G) shows quantification of GFP expression from the Western Blots shown in FIG. 2(F).

Consistent with the results obtained previously, incorporation of the US11 5′ leader did not affect levels of the GFP transcript in uninfected cells compared to HSV1 infected cells (as shown in FIG. 3H). FIG. 3H shows RT-qPCR quantification of LUC-GFP mRNAs from the experiment described hereinbefore. In the figure, two-way ANOVA with Sidak's post-hoc test was performed, wherein n=3 biological replicates and error bars indicate standard deviation (sd). * p<0.05, ** p<0.01, **** p<0.0001, ns, non-significant.

The US11 5′ Leader Enhances Transgene Protein Expression from Engineered HSV1 Virions

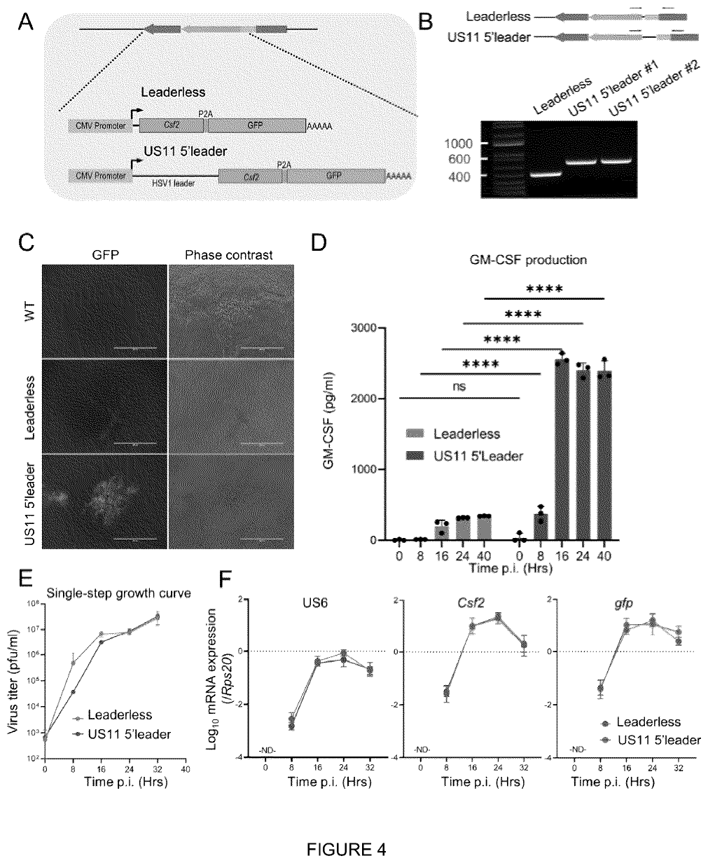

To validate the potential of the US11 5′ leader as a transgene enhancer, recombinant HSV1 strains were constructed based on the bicistronic pTK transgene expression plasmid described above. The linearized pTK plasmid was used to generate recombinant viruses following co-transfection with purified HSV1 genomic DNA. Homologous recombination of the expression cassette into the tk locus produces Δtk virus progeny expressing the transgene in a constitutive manner under the CMV promoter (as shown in FIG. 4A). As noted earlier, different promoters, translation enhancers and regulatory elements could be used along with or instead of CMV promoter.

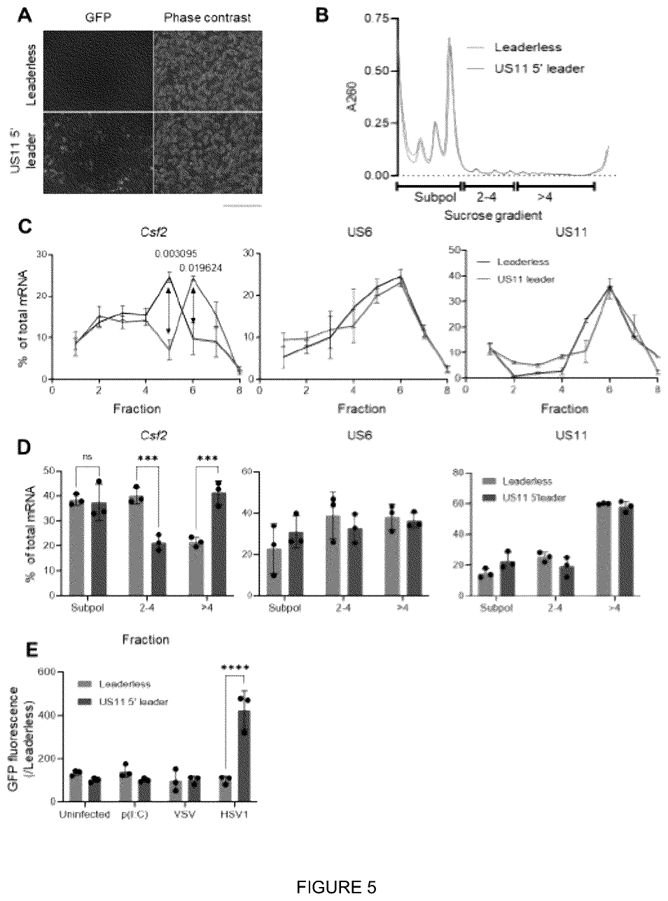

FIG. 4 shows that a recombinant HSV1 virus exhibits a US11 5′leader-dependent boost in GM-CSF expression. Specifically, FIG. 5A shows a schematic illustration of the expression cassette insertion scheme from the pTK-CSF2-GFP plasmid into HSV1 genome (note that the TK gene is on the minus strand), and the resulting transcripts expressed from the inserted cassette. In agreement with the enhanced expression of GFP conferred with the US11 5′ leader in the plasmid-based systems, plaques on Vero cells of HSV1 US11-Csf2 showed elevated GFP fluorescence compared to plaques of leaderless HSV1 Csf2 (as shown in FIG. 4C). FIG. 4C shows fluorescence imaging results of individual plaques of wild-type HSV1, HSV1 Csf2 construct and HSV1 US11-Csf2 construct.

To better demonstrate the clinical potential, the LUC ORF in the LUC-GFP expression cassette was replaced with the GM-CSF (Csf2) ORF, a gene that is virally expressed in the FDA-approved oncolytic HSV1. A leaderless (HSV1 Csf2) virus along with 2 virus clones incorporating the US11 5′ leader (HSV1 US11-Csf2) were constructed. FIG. 4(B) shows the results of virus genotyping for confirmation of expression cassette insertion. PCR was done using HSV1 gDNA extracted from purified viruses to confirm the insertion of the leaderless CSF2-GFP cassette (˜400 bp) and US11 5′ leader-CSF2-GFP cassette (˜600 bp) into the TK region of HSV1 genome.

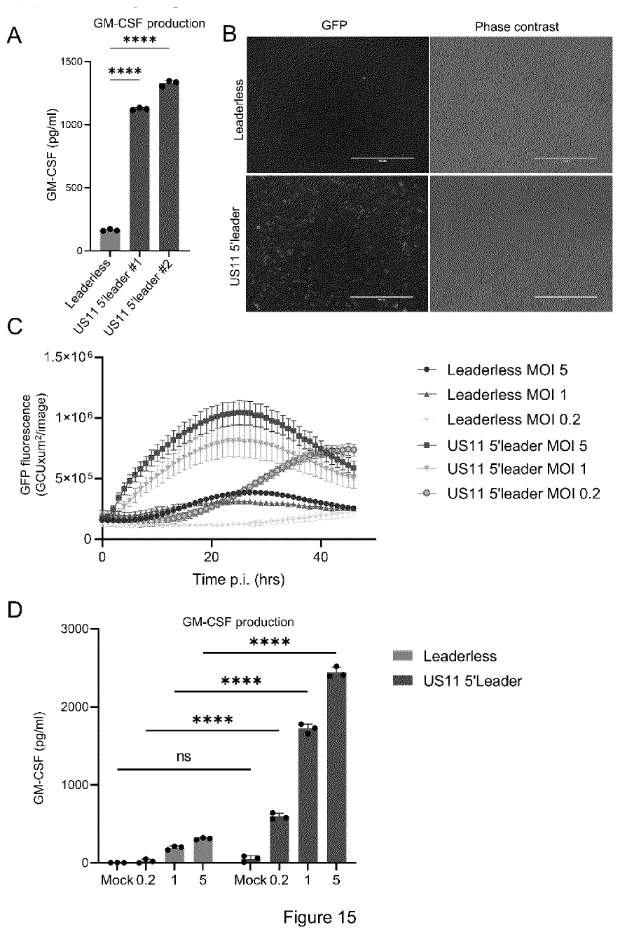

Cells infected with either of the HSV1 US11-Csf2 virus clones produced several-fold more GM-CSF compared to cells infected with HSV1 Csf2 (FIG. 4D, FIG. 15). FIG. 4D shows the quantification of GM-CSF production in culture supernatant of Vero cells infected with HSV1 expressing leaderless CSF2-GFP or US11 5′leader-CSF2-GFP. Monolayer of Vero cells was infected with the indicated virus at an MOI of 5, and the culture supernatant was collected 24 hours post-infection. GM-CSF concentration was quantified by ELISA. One-way ANOVA with Dunnett's post-hoc test was performed. n=3 biological replicates. Error bars: +sd. **** p<0.0001.

FIG. 15 characterizes expression enhancement by US11 5′ leader in oncolytic HSV1. Specifically, FIG. 15A shows monolayer of Vero cells was infected with the indicated virus at a MOI of 5, and the culture supernatant was collected 24 hours post infection. GM-CSF concentration was quantified by ELISA. One-way ANOVA with Dunnett's post-hoc test was performed. n=3 biological replicates. Error bars: +sd. **** «p<0.0001. FIG. 15B shows representative GFP fluorescence of HSV1 expressing Leaderless CSF2-GFP or US11 5′leader-CSF2-GFP. Monolayer of CT26 was infected with the indicated virus at an MOI of 5, then fluorescence microscopy images were taken at 24 hours post-infection. FIG. 15C shows time course of GFP fluorescence of HSV1 expressing Leaderless CSF2-GFP or US11 5′leader-CSF2-GFP. Monolayer of CT26 was infected with the indicated virus at an MOI of 0.2, 1 or 5, then GFP fluorescence was monitored over 2 days post-infection using the Incucyte live cell imaging system. FIG. 15D shows dose dependency analysis of secreted GM-CSF. CT26 cells were infected with the indicated virus at an MOI of 0.2, 1 or 5, then culture supernatant was collected 24 hours post-infection. GM-CSF concentration was quantified using ELISA. Two-way ANOVA with Sidak post-hoc test was performed. n=3 biological replicates. Error bars: +sd. **** p<0.0001.

In some instances the increase in production was almost ˜8 fold. Although, the highly increased protein expression was very promising, It was important to assess if the enhanced GM-CSF production was a result of higher viral replication. In order to ascertain that, the replication kinetics were measured by single-step growth curve and it was observed that all virus clones exhibited comparable replication kinetics (FIG. 4E). FIG. 4E shows results of the single-step growth curve of HSV1 Csf2 and HSV1 US11-Csf2. Monolayers of Vero cells were infected at an MOI of 5, then intracellular and extracellular virus was collected and titrated at the indicated time points.

RT-qPCR of mRNAs extracted from infected cells at various time points revealed that the presence of the 5′ leader did not affect the expression kinetics of the HSV1 transcript US6 (as shown in FIG. 4F left panel) nor that of the transgene transcripts expressed in cis (Csf2 and GFP), FIG. 4F middle and right panel) over the course of infection. FIG. 4F shows transcription levels of HSV1 endogenous gene (US6) and transgenes. More specifically, monolayers of Vero cells was infected at an MOI of 5, then cells were lysed using Trizol at the indicated time points, finally mRNA abundance was quantified by RT-qPCR and normalized to Rps20. ANOVA, analysis of variance. MOI, multiplicity of infection.

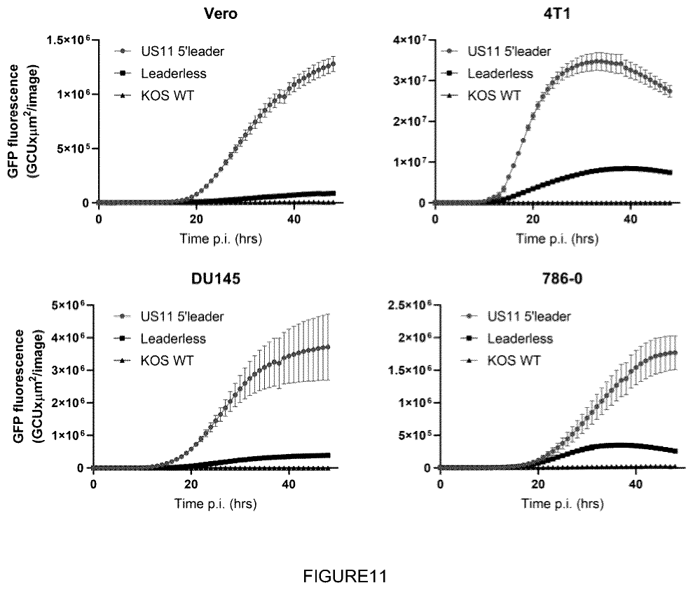

In order to understand whether the transgene enhanced protein production might be a cell-type or species-dependent effect, the human prostate cancer line DU145 and the human renal cell carcinoma line 786-O were infected with wildtype, leader, and leaderless viruses. Monitoring of GFP fluorescence intensity in these cells over the course of infection revealed a robust US11 5′ leader-mediated enhancement of transgene protein expression in all cell lines tested (FIG. 11). It was observed that US11 leader enhancement is robust in different cell types and species. FIG. 11 shows experimental results of a monolayer of the African green monkey kidney cell line Vero, mouse breast cancer cell line 4T1, human pancreatic cancer cell line DU145 and human renal carcinoma cell lines 786-O, where the cell lines were infected with HSV1 KOS leaderless or HSV1 US11 5′leader at an MOI of 0.1. GFP fluorescence intensity was observed for 48 hours using the Incucyte live cell imaging system. These results demonstrate the ability of the US11 5′ leader to augment the production of a clinically relevant therapeutic transgene in HSV1-infected mammalian cells. A person skilled in the art would appreciate that the above example is in no way limiting and similar results would be observed if other cell lines or strains were employed.

It is therefore concluded that 5′ leader of the HSV1 late gene US11 is capable of increasing translation of a downstream cistron in heterologous reporter constructs and, notably, this effect was only observed with concomitant HSV1 infection. As noted earlier, HSV1 virus engineered to express a cassette containing the US11 5′ leader upstream of the GM-CSF ORF, conferred superior GM-CSF expression compared to its leaderless counterpart in several mammalian cell lines and most importantly in a mouse model of cancer resulted in improved anti-tumour efficacy and prolonged survival. This confirms that it is possible to enhance therapeutic payload expression from oncolytic HSV1 platforms by incorporation of a viral 5′ leader into the expression transgene cassette.

### The HSV1 US11 5′ Leader Increases Translational Efficiency of Associated Transcript in an HSV1 Dependent Manner

Without wishing to be bound by theory, the inventors sought to identify the mechanism underlying the increased protein expression in cells infected with HSV1 US11-Csf2. To assess the effect of the US11 5′ leader on mRNA translation of the transgene, the polysome profiling technique was employed. Briefly, ribosome-bound mRNAs were separated by ultracentrifugation on a sucrose gradient, which caused mRNAs to sediment based on the number of bound ribosomes. Accordingly, mRNAs that migrated towards heavier sucrose gradient had more bound ribosomes and possessed higher translation efficiency. Vero cells were infected with HSV1 Csf2 or HSV1 US11-csf2 at an MOI of 5 (FIG. 5A), then the lysate containing ribosome-bound mRNAs was resolved on a 10-50% sucrose gradient (FIG. 5B). FIG. 5A shows the fluorescence and phase contrast images of HSV1 infected Vero cells used for the polysome fractionation experiment in 5B and 5C at a scale bar of 400 μm. FIG. 5B shows polysome traces of Vero cells infected with HSV1 Csf2 or HSV1 US11-Csf2 at an MOI of 5, wherein cells were lysed at 24 hours post-infection for polysome fractionation.

It was observed that the presence of the US11 5′ leader caused a shift of the Csf2-gfp transcript distribution toward heavier polysome fractions, demonstrating enhanced translation efficiency compared to the leaderless Csf2-gfp transcript (as seen in FIGS. 5C and 5D). FIG. 5C shows mRNA distribution in polysome fractions of Csf2 (as shown in the upper panel) and the endogenous HSV1 transcripts US6 (as shown in the middle panel) and US11 as shown (as shown in the lower panel), quantified by RT-qPCR, wherein two-sided t-test was performed, where n=3 biological replicates and the error bars indicate standard deviation. **: p<0.01, *: p<0.05. FIG. 5D shows mRNA distribution in non-translating fraction (subpolysome), poorly translating fraction (2-4 ribosomes) and highly translated fraction (>4 ribosomes) of Csf2 (as shown in the upper panel), US6 (as shown in the middle panel) and US11 (as shown in the lower panel) transcripts. Multiple unpaired t-tests were performed, wherein n=3 biological replicates and error bars indicate standard deviation (sd).***: p<0.00.

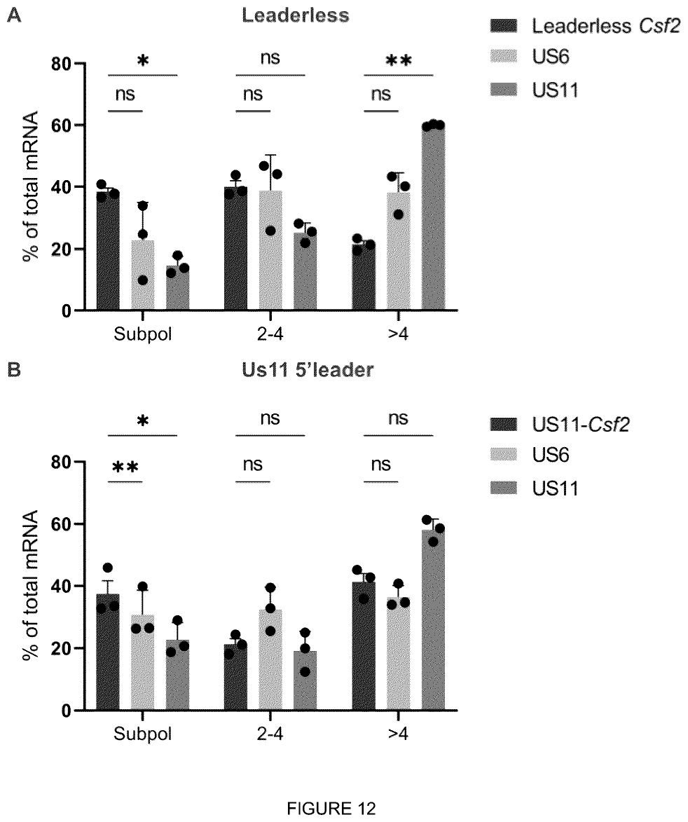

Interestingly, it was observed that both the US6 and US11 viral mRNAs mostly distributed to the heavier polysome fractions (as shown in FIGS. 5C and 5D), suggesting that HSV1 transcripts are generally highly translated despite the global shutoff of protein synthesis resulting from HSV1 infection. Notably, without US11 5′ leader, the transgene Csf2-gfp mRNAs were suboptimally translated compared to the US6 and US11 viral mRNAs (as shown FIGS. 12A and 12B). FIGS. 12A and 12B shows that leaderless transgene mRNA are poorly translated compared to viral mRNA. In the same polysome profiling experiment described in FIG. 5, the mRNA distribution of US6 and US11 were compared to that of leaderless Csf2 (shown in FIG. 12A) or US11-Csf2 (shown in FIG. 12B) mRNA This data suggests that traditional transgene cassettes that lack cis-acting translation enhancing elements are poorly translated when compared to HSV1 endogenous transcripts. The experiment also demonstrates that incorporation of the HSV1 US11 5′ leader significantly improves translation of a transgene in HSV1 infected cells.

In order to clarify if the translation enhancement mediated by the US11 5′ leader is specific for HSV1 infected cells or a result of a general antiviral state, GFP expression from the plasmid pTK-Csf2-gfp in Vero cells was transfected with the dsRNA mimic poly(I: C) or infected with another virus, such as VSV. It was observed that neither poly(I: C) transfection nor VSV infection were capable of inducing GFP expression (as shown in FIG. 5E). FIG. 5E shows quantification of GFP fluorescence of Vero cells transfected with pTK-CSF2-GFP plasmid with or without US11 leader sequence, co-transfected with poly(I: C) or infected immediately after with VSV or wild-type HSV1 at an MOI of 5. Thus, the enhancement of US11 5′ leader on gene expression appears to be specific to HSV1 infected cells. These data suggest that traditional transgene cassettes that lack cis-acting translation enhancing elements are suboptimally translated when compared to HSV1 endogenous transcripts. The data also demonstrates that incorporation of the HSV1 US11 5′leader can significantly and specifically improve translation of HSV1-encoded transgenes.

### The US11 5′ Leader Improves the Antitumour Effect of GM-CSF Expressing HSV1 In Vivo

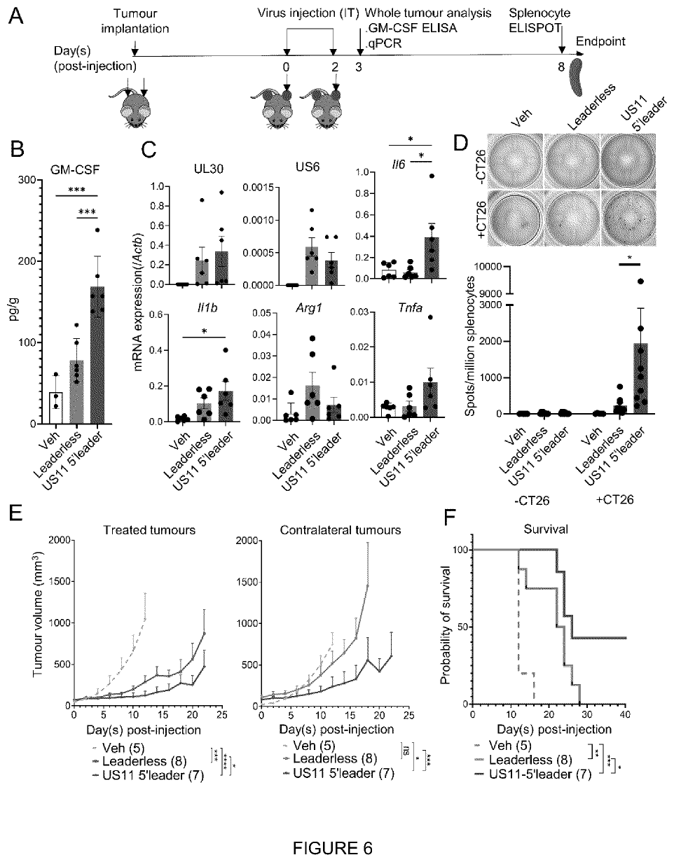

The inventors further investigated whether boosting the expression of a transgene beyond that achieved by current oncolytic HSV1 platforms is capable of ameliorating cancer outcomes. In order to assess this, the CT26 syngeneic tumour model of colon carcinoma that is routinely used to assess the efficacy of oncolytic HSV1. Tumours were generated on both flanks of the mice, and tumours on one side were injected with HSV1 Csf2 or HSV1 US11-Csf2 viral particles. The contralateral tumour was injected with virus resuspension buffer (as shown in FIG. 6A). Tumours were generated on both flanks of the mice, and tumour on one side was injected with resuspension buffer or 5×10⁵ viral particles of HSV1 leaderless-Csf2 or HSV1 US11-Csf2. FIG. 6 shows that US11 leader sequence enhances the antitumour effect of GM-CSF expressing oncolytic HSV1. FIG. 6A shows the schematic representation of the in vivo study design, wherein 10⁵ CT26 cells were injected in both flanks of BALB/c mice. When the tumour reached approximately 5×5 mm, two injections of 5×10⁵ PFU of the indicated virus were performed intratumourally two days apart (Day 0 and Day 2) and tumour size were measured every 2 days.

Analysis of the injected tumours confirmed that incorporating the US11 5′ leader enhanced intratumoural GM-CSF expression in tumours treated with HSV1 US11-Csf2 (as shown in FIG. 6B), while both viruses share similar replication kinetics in vivo as shown by comparable transcription levels of the viral genes US6 and UL30 (as shown in FIG. 6C). This observation suggested that both viruses similarly infect tumour cells, but increase in GM-CSF production was only observed in HSV1 US11-Csf2 infected cells. FIG. 6B shows intratumoural GM-CSF level of tumour treated with Leaderless or HSV1 US11-Csf2. Tumours as generated in 6A were excised one day after the second injection and homogenized in PBS, following which GM-CSF level was then quantified by ELISA. Two-sided t-test was also performed, wherein n=3 biological replicates and error bars indicate standard deviation (sd).

FIG. 6C shows results of HSV1 replication in tumour, as measured by viral transcript expression levels. More specifically, RNA from tumours in 5B were extracted with Trizol, then mRNA abundance of the indicated transcripts were quantified by RT-qPCR and normalized to Actb. Two-tailed unpaired t-test was also performed, where n=3 biological replicates and error bars indicate standard deviation (sd).

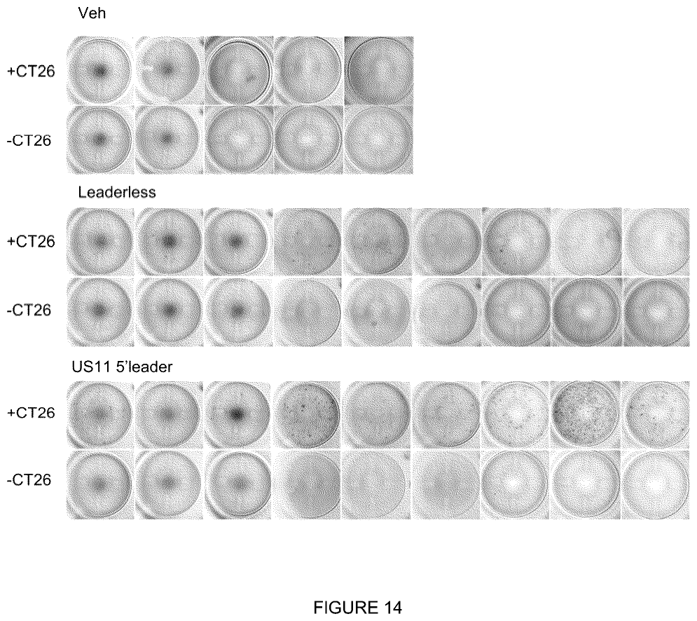

GM-CSF is known to be a pro-inflammatory cytokine, but it may exert anti-inflammatory properties in certain contexts. Upon probing the tumour microenvironment of the infected tumours by analyzing mRNA levels of representative inflammatory genes, elevated level of II1b, 116 and Tnfa mRNAs in tumours treated with HSV1 US11-Csf2 was observed (FIG. 6D). FIG. 6D shows the expression of representative inflammatory genes in injected tumours. RNA from tumours in 6B were extracted with Trizol, then mRNA abundance of the indicated transcripts were quantified by RT-qPCR and normalized to Actb. The systemic anti-tumour response was also analyzed on the eighth day after the first injection by IFNγ ELISPOT on splenocytes co-cultured with UV-irradiated CT26 cells. No CT-26-specific immune cell response was found in the spleen of vehicle-treated mice, while the leaderless HSV1 was capable of inducing certain level of CT26-specific immune cells. However, the HSV1 US11-Csf2 induced a significantly higher CT26-specific T-cell response compared to the leaderless virus (FIG. 6D, FIG. 14)

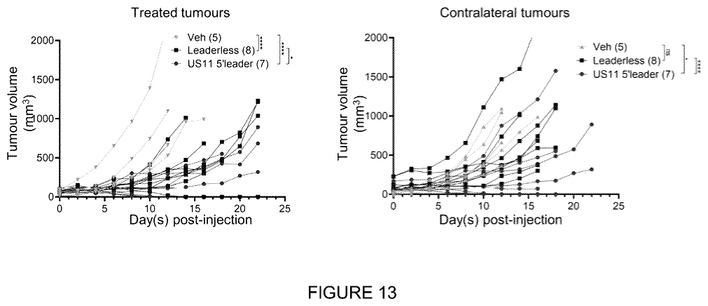

Finally, the anti-tumour effect of both viruses (FIG. 6E, FIG. 13) were directly compared. As expected, regardless of viral clone, HSV1-injected tumours showed decreased tumour growth relative to vehicle-injected tumours (FIG. 6F), a result consistent with the oncolytic and immunomodulatory properties of this viral platform. Importantly, the growth of tumours injected with the US11 5′leader virus was significantly lower than that of tumours injected with the leaderless virus (FIG. 6F, left panel). More interestingly, treatment with the leaderless virus had no significant effect on the contralateral tumours, while treatment with the US11 5′leader virus significantly slowed tumour growth on the contralateral side, comparable to that observed in the treated tumour (FIG. 6F, right panel); suggesting an abscopal effect consistent with the elevated anti-tumour immune response observed by ELISPOT. Finally, we found that the US11 5′leader virus significantly improved mouse survival (FIG. 6). Collectively, these data demonstrate that increasing the expression of GM-CSF via incorporation of the translation-enhancing US11 5′leader augments intratumoural cytokine production and boosts anticancer efficacy in a pre-clinical colon cancer model.

This observation suggests that HSV1 US11-Csf2 can induce a more inflammatory tumour microenvironment, even at the same treatment dose and growth rate as the Leaderless virus. Finally, the antitumour effect of both viruses was compared directly. As expected, HSV1-injected tumours, regardless of viral clone, showed decreased tumour growth relative to vehicle-injected tumours (as shown in FIG. 6E), an observation consistent with the oncolytic and immunomodulatory properties of this viral platform. FIG. 6E shows the effect of Leaderless- or HSV1 US11-Csf2 treatment on tumour growth and the number of mice are shown in brackets. ANOVA with Sidak's post-hoc test was also performed where the error bars are ±sd (standard deviation). FIG. 13 shows the size of individual tumours shown in FIG. 6E.

The growth of tumours injected with the US11 5′ leader virus was significantly lower than in tumours injected with the leaderless virus (as shown in FIG. 5E, left panel). More interestingly, treatment with the leaderless virus failed to show a significant effect compared to vehicle on the contralateral tumour, yet treatment with the US11 5′ leader virus showed a much slower tumour growth profile, comparable to that observed in the ipsilateral tumour (as shown in FIG. 6E, right panel); suggesting an abscopal effect. Finally, we found that the presence of the US11 5′ leader significantly improved mouse survival with the US11 leader virus, consistent with its superior expression of GM-CSF (as shown in FIG. 6F). FIG. 6F shows results of Kaplan-Meier survival curve of mice treated with the Leaderless- or US11-Csf2 HSV1, where the number of mice are shown in brackets.

Collectively, the above experimental data demonstrated that increasing the dose of GM-CSF via incorporation of the translation-enhancing US11 5′ leader improved intratumoural cytokine production to boost anticancer efficacy in a preclinical colon cancer model.

In view of the discussion and experimental data discussed hereinbefore, it is evident that incorporating a HSV1 5′leader sequence enhances downstream transgene protein expression from a recombinant HSV1 virus. It is hypothesized that the elevated expression is mediated through increased mRNA translation of the modified transgene transcript and within infected cancer cells. It was also observed that an oncolytic HSV1 harboring a 5′leader upstream of a therapeutic transgene has superior antitumour activity compared to a leaderless HSV1. This approach represents a simple yet highly effective way to improve the current generation of oncolytic HSV1 platforms that are presently in clinical trials. This strategy can also be used as a complementary technique to those that employ either a strong heterologous promoter to drive transgene expression or an approach that inserts the transgene into a highly transcriptionally active region of the HSV1 genome. As an example of the former strategy, Toda et al. found that a cassette inserted into the TK region and expressing GM-CSF driven by a CMV promoter yielded approximately 55 μg of GM-CSF per 10⁵ Vero cells. In the present study, which also employed a CMV-driven, GM-CSF expression cassette inserted in the TK region of the HSV1 genome, we saw 41.75±2.35 μg GM-CSF per 10⁵ Vero cells, a value very close to that previously reported (as shown in FIG. 3D, FIG. 15; which was calculated based on 167±9.4 μg/ml of GM-CSF observed in a 12-well plate format at confluency) which typically contains 4×10⁵ cells in 1 ml of culture media). Yet, with the introduction of the US11 5′leader, close to an 8-fold increase in GM-CSF production was obtained, representing a significant improvement in transgene protein expression. It is possible that transgene production can be improved even further by combining a strong HSV1 promoter (e.g, the one driving HSV1 RL2 expression) with a translation enhancer such as the US11 5′leader.

It was observed that the enhanced translation of Csf2 transcript expressed from a HSV1 backbone improved antitumour efficacy in a dual transplanted flank tumour mouse cancer model. As expected, tumours treated with the leaderless HSV1 had slower progression and the corresponding mice had a better survival rate compared to the mock-treated counterparts (as shown in FIGS. 6E and 6F). However, the HSV1 US11-csf2 virus not only inhibited tumour growth to a higher extent in the injected tumours, but also induced significant inhibition of tumour growth in the contralateral tumours, and resulted in an improved survival rate. Consistently, it was observed that a higher intratumoural GM-CSF concentration in tumours from mice administered with the US11 5′leader HSV1, which also correlated with upregulation of inflammatory gene markers (as shown in FIGS. 6B and 6D), and elevated levels of tumour-specific immune cells in the spleens of treated mice (FIG. 6D), suggesting a modification of the tumour microenvironment towards a desired inflammatory milieu that resulted from an augmented anti-tumour immune response. As GM-CSF was shown to enhance systemic antitumour immune responses, the results indicate that an increase in GM-CSF production can be sufficient to induce a more inflammatory tumour microenvironment in the treated tumours, and lead to an improved systemic antitumour immune response against distant tumours. Overall, the results show that despite the use of a strong promoter (CMV promoter), alone does not maximize transgene protein expression, such as by incorporating a translation enhancer, can boost the payload levels further, leading to increased oncolytic virus efficacy.

The following section provides more information on the materials and how they were prepared or obtained.

Cell culture and viruses: The mouse breast cancer cell line 4T1, mouse colon cancer carcinoma CT26, human prostate cancer DU145, human renal carcinoma 786-O, HEK293T and Vero cells were acquired from American Tissue Culture Collection. 4T1 was maintained in Roswell Park Memorial Institute (RPMI) 1640 (Fisher) supplemented with 10% fetal bovine serum (FBS) (Sigma-Aldrich) and 1X penicillin/streptomycin (Fisher). HEK293T and Vero cells were maintained in Dulbecco's Modified Eagle Medium (DMEM) (Fisher) supplemented with 10% FBS and 1X penicillin/streptomycin (Fisher). Cells were incubated at 37° C., 5% CO2 v/v. All HSV1 strains were propagated on Vero cells. Monolayer of Vero cells was inoculated with HSV1 at 0.1 MOI, then culture for approximately 24-48 hours until close to 100% cytopathic effect was observed. Supernatant was then collected separately, and infected cells were freeze-thawed three times to release intracellular virus. Both the cultured supernatant and the freeze-thawed lysate were clarified by centrifuged at 1000 g, 5 min to remove cell debris. The supernatants were combined and filtered through a 0.45 μm filter. Virus particle was further purified using sucrose cushion by overlaying the supernatant on top of a 36% sucrose cushion in PBS and centrifuge at 18,000 g for 2 hours, 4° C. Virus in the pellet was resuspended in HNE buffer (HEPES 10 mM, NaCl 150 mM, EDTA 0.1 mM, pH 7.2) and stored at −80° C.

RNA-Seq mapping and TSS identification: RNA-Seq data of 4T1 cells infected by HSV1 was previously published. For mapping of RNA-seq, RNA reads were mapped to the HSV1 reference genome JQ780693.1 using HISAT262. Only one copy of the two flanking inverted repeat regions were used: the TRL region 1-8870 and TRS region 144602-151023 were omitted. Transcription start site (TSS) were manually identified from RNA read coverage, defined by an abrupt increase of read coverage at a base position (shown is FIG. 2). The leader sequence was defined as the sequence from the TSS to the annotated start codon of the associated HSV1 gene, excluding any spliced intron if applicable. For converting leader sequence coverage between different HSV1 reference genomes (JQ780693.1 to JN555585.1), the sequence of the identified leader from JQ780693.1 was aligned to JN555585.1 using NCBI-BLAST (https://blast.ncbi.nlm.nih.gov/Blast.cgi) to find the corresponding coordinates on JN555585.1. Raw data was deposited and analyzed on the Galaxy server (https://galaxyproject.org). Gene expression level from mapped RNA-seq and Ribo-seq reads was calculated using Cuffdiff.

Plasmid construction: For the leader translation activity screen using the CAT reporter assay, virus leader sequences were amplified by PCR from cDNA reverse-transcribed from mRNA of HSV1 infected 4T1 cells. Briefly, total RNA was extracted from cell using TRIzol reagent (Fisher), treated with Turbo DNA-free kit (Thermo Fisher) to remove potential contamination of virus genomic DNA, then reverse-transcribed by using the iScript Advanced cDNA Synthesis Kit (BioRad). The forward and reverse primers (Notl_5′UTR-F and Xhol_5′UTR-R) of each leader also contain the restriction sites of Notl and Xhol, respectively, for subsequent cloning. Amplification specificity was confirmed for each pair of primer by including a negative control cDNA from mRNA of uninfected 4T1. The PCR amplicons were cloned into the CAT reporter plasmid pMCpA using the Notl-Xhol restriction sites. A leaderless CAT reporter construct, in which only a short residual sequence from the plasmid MCS is transcribed with the CAT CDS, was used as control (this residual sequence also present in all viral 5′leader construct, directly 5′ upstream of the leader sequence). For inserting transgene expression cassette into HSV1 genome, the cassette was cloned into the pTK-Green plasmid, flanked by two regions of the HSV1 TK gene to allow for homologous recombination. The expression cassette consists of the leader followed by the transgene (firefly luciferase or mouse GM-CSF), the self-cleaving peptide porcine teschovirus-1 2A (P2A) and GFP. The insert was generated by fusion PCR. The virus leader was amplified as described above using forward primer containing a AgeI cutting site (AgeI_5′UTR-F) and a reverse primer with an overlap section of the transgene 5′end (5′UTR_LUC-R or 5′UTR_CSF2-R). The second fragment containing the transgene CDS was amplified using a forward primer (5′UTR_LUC-F or 5′UTR_CSF2-F) and a reverse primer consisted of the CDS 3′ end, a GSG linker and a part of the P2A sequence (LUC-GSG-P2A-R). The third fragment containing the GFP was amplified using a forward primer (GSG-P2A-GFP-F) and a reverse primer that include a Kpnl cutting side (GFP-Kpnl-R). All three fragment were purified using the QIAquick PCR Purification Kit (Qiagen), then use as templates for fusion PCR using the 5′-most (AgeI_5′UTR-F) and 3′-most primer (GFP-Kpnl-R). The resulting PCR product was cloned into the pTK-Green using AgeI and Xhol sites. All plasmids were verified by Sanger sequencing. A leaderless LUC-GFP or CSF2-GFP construct, in which only a short residual sequence from the plasmid MCS is transcribed with the transgene CDS, was used as control (this residual sequence was also present in all viral 5′leader constructs, directly 5′ upstream of the leader sequence). All primer sequences are presented in Table S1.

TABLE S1
Oligonucleotides used in this study (Hoang et al.)

| Primer Name | Primer sequences (5′-3′) | Description | Source |
|---|---|---|---|
| [symbol] | [symbol] | [symbol] | This study |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | [symbol] | This study |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| | [symbol] | | |
| [symbol] | [symbol] | | |
| [symbol] | [symbol] | | |
| [symbol] | [symbol] | | |
| [symbol] | [symbol] | | |
| [symbol] | [symbol] | qPCR | [symbol] |
| [symbol] | [symbol] | | [symbol] |
| [symbol] | [symbol] | | This study |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |
| [symbol] | [symbol] | | |

[symbol]

indicates data missing or illegible when filed

CAT reporter assay: CAT reporter assay was performed as previously described. Cells were seeded at approximately 75% confluency in a 6-well plate and incubate for one day before transfection. In the case of HSV1 infection, cells were infected with HSV1 at 5 MOI 1 hour before transfection. CAT reporter plasmid was co-transfected with β-Galactosidase plasmid at 1 μg per plasmid using Lipofectamine 2000 (Thermofisher) according to the manufacturer's protocol. Cells were lysed 24 hours post transfection and assayed for CAT expression using the CAT ELISA kit (Roche). β-Galactosidase activity was also measure from lysate using ortho-Nitrophenyl-β-galactoside (ONPG) colorimetric assay. CAT expression was normalized to β-Galactosidase activity to control for transfection efficiency.

Quantitative RT-PCR: DNase-treated RNA and cDNA were prepared as described above. For RT-qPCR, SsoAdvanced Universal SYBR Green supermix (BioRad) was used with a CFX96 Touch Real-Time PCR Detection System (BioRad). The PCR condition was 95° C. for 3 minutes, followed by 40 cycles of 95° C. for 10s and 60° C. for 30s, and ended with a standard melting curve cycle. Gene expression was calculated using the ΔΔCt method against the indicated reference genes. The list of primer for the genes or sequences of interest is in Table S1.

Western Blot: Cells were washed once with 1×PBS, then lysed on ice using RIPA buffer (150 mM NaCl, 1.0% IGEPAL® CA-630, 0.5% sodium deoxycholate, 0.1% SDS, 50 mM Tris, 50 mM NaF, 15 mM NaVO3, pH 8.0) supplemented with complete Protease Inhibitor Cocktail (Roche). Lysate were centrifuge at 10,000 g, 10 min at 4° C. to remove cell debris. Protein concentration was determined using the DC Protein assay kit (BioRad). An indicated amount of total protein was used for SDS-polyacrylamide gel electrophoresis (PAGE) using 10% SDS-polyacrylamide gel. Separated protein was transferred to PVDF membrane, the membrane was blocked with 5% w/v skim milk in TBS-T buffer (10 mM Tris, 50 mM NaCl, 0.1% Tween-20, pH 7.5) and then blotted for the indicated antibody. The following antibodies and corresponding dilution were used: anti-GFP (Abclonal, CAT #AE011) at 1:2000, anti-β-actin (Sigma, #A5441) at 1:10,000, IRDye® 800CW Goat anti-Mouse IgG Secondary Antibody (LICOR, CAT #926-32210) at 1:20,000 and IRDye® 680RD Goat anti-Rabbit IgG Secondary Antibody (LICOR, CAT #926-68071) at 1:20,000.

Live-cell monitoring of GFP expression: Live-cell monitoring of GFP expression was performed using the IncuCyte Live-Cell. Sartorius was used as the monitoring system. Transfection and/or infection was performed as indicated, then cell plates were placed insides the IncuCyte system and cultured and monitored at 37° C., 5% CO2 with phase contrast and fluorescent images taken every 2 hours. Images were the analyzed with the IncuCyte ZOOM software using the following parameter: background subtraction using Top-Hat method (disk shape structuring element with radius of 10 μm, threshold of 1.0 green calibrated unit), edge split: Off, Hole Fill: No, Adjust Size: No, Filters: No.

Generating recombinant HSV1 viruses: For inserting the expression cassette into HSV1 genome, the TK gene was targeted for insertion as previously described. HSV1 genomic DNA was extracted from purified virus stock using the QIAamp DNA mini kit (Qiagen). HEK293T cells were seeded at 75% confluency one day before in 6-well plates, and then co-transfected with HSV1 gDNA: pTK-Green plasmid at a ration of 1:40, for a total amount of 1 tg DNA/well, using Lipofectamine 2000 (Thermo Fisher) according to manufacturer protocol. Cells were then cultured for 3-5 days until cytopathic effect was observed. Cells were then freeze-thawed three times, centrifuged at 1000 g, 5 min to remove cell debris and supernatant was collected. Supernatants at various dilutions were then inoculated to monolayer of Vero cells in order to separate individual plaques overlaid with DMEM supplemented with 10% FBS and 1% carboxymethylcellulose (CMC) to allow for development of individual plaques. GFP-positive plaques were selected and subjected to multiple plaque purification rounds until a pure GFP-expressing HSV1 population was obtained. Insertion of the cassette into HSV1 genome was confirmed first by PCR genotyping using a forward primer on the TK gene (pTK-seq) and a reverse primer on the transgene (CSF2.e-R), followed by Sanger sequencing.

Plaque titration: Virus stock or solution were serially diluted, then inoculated a monolayer of Vero cells and incubated for 1 hours, 37° C., 5% CO2 with frequent shaking. The virus-containing media was then removed and an overlay of DMEM+10% FBS+1% agar was added. Cells were then cultured at 37° C., 5% CO2 until visible plaques could be observed using a brightfield microscope. Plaques were visualized using crystal violet staining and counted.

Polysome fractionation: Polysome fractionation was performed as previously described. Briefly, cells were treated with cycloheximide (CHX) (Bioshop, CAT #66-81-9) at 100 μg/ml for 5 min to stop ribosome, washed three times with ice cold PBS supplemented with CHX (100 μg/ml) and lysed using polysome lysis buffer (5 mM Tris pH 7.5, 2.5 mM MgCl2, 1.5 mM KCl, 100 μg/ml CHX, 2 mM DTT, 0.5% Triton X-100, 0.5% sodium deoxycholate) supplemented with 100 units RNAsin Ribonuclease inhibitor (Promega). Cell debris was cleared by centrifugation at 14,000 g for 10 min, 4° C. Supernatant was then loaded on a 10%-50% continuous sucrose gradient and centrifuge at 36,000 rpm, 90 min at 4° C. in a SW41Ti rotor. Fractions were then collected and the OD260 absorbance of fractions was monitored using a Brandel Fraction Collector System (Brandel). RNA was extracted from each fraction using TRIzol reagent (Thermo Fisher) according to the manufacturer's protocol.

GM-CSF quantification: To measure GM-CSF production from engineered HSV1 infection, cells were seeded at 80-90% confluency and then infected with the indicated HSV1 at an MOI of 5. 24 hours post-infection, culture supernatant was collected and GM-CSF production was measure using the Mouse GM-CSF ELISA Kit (CSF2) (Abcam, CAT #ab100685) according to the manufacturer's protocol.

Single step-growth curve and monitoring HSV1 genes expression: For monitoring virus replication and viral gene transcription during a single step growth curve, cells were seeded at 80-90% confluency and infected the next day at an MOI of 5. Both cells and culture supernatant were collected at the indicated time points and used for virus titration by plaque assay, or RNA extraction for quantification of viral transcript expressions as described above.

CT26 subcutaneous tumour model: Female BALB/c were ordered from Charles River (Kingston, NY, USA). Animals were received at 5-6 weeks old, housed at 5 per cage, were fed ad libitum, and acclimated to the facility for two weeks before experimental manipulation. For tumour implantation, 10⁵ CT26 cells were injected subcutaneously into both flanks of the mouse. When the tumour is palpable (˜5×5 mm), tumour on one flank was injected twice, one day apart, intratumourally with 50 μl DMEM or 5×10⁵ PFU of the indicated virus, while the tumour on the other flank was left untreated (contralateral). Tumour sizes were measured every two days using a caliper. Animals were euthanized when reached humane endpoint or individual tumour reached 2000 mm³, or at an alternate humane endpoint. The in vivo study was done single-blinded: the animal handler did not know the treatment given to each mice group. For assessing intratumoural GM-CSF level and HSV1 transcript abundance, tumours were excised, then finely chopped and homogenized in PBS buffer using 2.0 mm zirconia beads (Thomas Scientific, CAT #1197P96) with a TissueLyzer II (QIAGEN) at 20 Hz/s. Half of the homogenate was used to for mouse GM-CSF was measured using the Mouse GM-CSF ELISA Kit (CSF2) (Abcam, CAT #ab100685) according to the manufacturer's protocol. The other half of the homogenate was used for RNA extraction with Trizol Reagent (Thermo Fisher), then transcript mRNA was quantified by RT-qPCR as described above.

IFNγ ELISPOT assay: Splenocytes were isolated freshly from spleens of mice 8 days after the first injection and cultured in RPMI (Fisher) supplemented with 10% FBS (Sigma-Aldrich) and 1X penicillin/streptomycin (Fisher). IFNγ ELISPOT was done using the mouse interferon-gamma ELISPOT kit (Abcam, CAT #ab64029) according to the manufacturer's protocol. Briefly, 100,000 splenocytes were co-cultured with/without 50,000 UV-irradiated CT26 in the ELISPOT well for 24 hours. Cells were then thoroughly washed away from the well, and IFNγ spots from stimulated T-cells were developed. Individual wells were imaged using the stereomicroscope LEICA EZ4 W, and spots were counted manually.

Data and Code Availability: The RNA-seq data was published previously and are available on the NCBI Gene Expression Omnibus (GEO: GSE137757), sample IDs GSM4086602 and GSM4086610 (HSV1 infected mRNA replicate).

Statistical analyses: All experiments were performed in at least three biological replicates. Statistical analyses were performed using GraphPad Prism 8, using the method indicated in figure legends. Error bars indicate standard error of the mean (SEM). * p<0.05, ** p<0.01, *** p<0.001, **** p <0.0001, ns, non-significant.

### Discussion and Analysis

The findings presented hereinbefore identified cis-acting sequences within virus-based therapies that can be exploited to enhance transgene expression. The method was first conceptualized by the comprehensive annotation of viral 5′leader sequences through TSS identification by RNA-Seq and mapping translational efficiency across a viral genome by ribosome profiling. In this way, the TSS of 61/73 genes of HSV1 were identified. During the lytic cycle, HSV1 expresses its genes in an orderly manner, classified into immediate-early (IE), early (E), and late (L) genes. A small number of selected 5′ leaders that are highly expressed in the late stages of HSV1 infection were primarily focused upon and as they might be better tuned to enhance translation at the last stage of infection (as shown in FIG. 2). Although the application primarily focuses on the US11 5′leader, but a person skilled in the art would be able to comprehend that there might be other specific viral sequences with potentially superior translation enhancing activity at a different time of infection that could be revealed with a more comprehensive screen of all HSV1 5′ leaders.

### Potential Mechanism of US11 5′Leader-Mediated Translation Enhancement During Infection

The most extensively studied translation enhancer elements of virus origin are IRESs, which support cap-independent translation of viral transcripts. Viral proteins can also act in-trans with 5′leader sequences to modify translational efficiency of a viral mRNA. In this regard, the HSV1 protein VHS was shown to modulate translation activity of certain host 5′ UTRs, as well as HSV1 viral sequences in a cap-independent manner. VHS is a major translation modifier protein of HSV1 and it possesses RNase activity towards ssRNA and interacts with the translation initiation complex, thus mediating the degradation of actively translating mRNAs in infected cells. During the late stage of HSV1 infection, VHS activity is attenuated by the viral proteins VP16 and VP22, allowing for productive translation of viral transcripts in a cap-dependent mode. The cap-independent mode of translation employed by the 5′leaders mentioned above might confer an advantage for the associated transcripts in the earlier phase of HSV1 infection.

Another HSV1 translation modifier is ICP27, which is crucial for translation of viral mRNAs via facilitating their nuclear export. ICP27 contains a RGG motif at its N-terminal that binds to viral transcripts via GC-rich regions, and links the transcripts to the host nuclear export complex via its interaction with the nuclear export factor REF and NXF1. While this study was limited to HSV1 5′leaders, it is possible that the same strategy may work for other viral vectors and OV backbones as well. In poxvirus, for instance, non-templated poly(A) sequences are added to the 5′leader of late viral transcripts due to viral polymerase slipping, which promotes translation of viral RNAs and this activity is attributed to the phosphorylation of the small ribosomal protein RACK1 by the virus kinase B160. Recently, it has been shown that the 5′leader of SARS-COV-2 sgRNA as well as gRNA also protects viral transcripts from translation shutoff by the viral NS1 protein. Thus, viral leaders are likely enriched in cis-regulatory sequences that have co-evolved with viral trans-acting protein factors to control viral gene expression post-transcriptionally, yet current virus-based therapeutic applications do not effectively employ them.

The inventors has thus shown that incorporating a HSV1 5′leader sequence enhances downstream transgene protein expression from a recombinant HSV1 virus. The experiments tested for transgenes that are intracellular (CAT, LUC, GFP) or secreted (GM-CSF), and expression levels were consistently induced by the incorporation of the US11 5′leader during HSV1 infection. The elevated expression was mediated through increased mRNA translation of the modified transgene transcript within infected cancer cells. Importantly, it was found that an oncolytic HSV1 harboring a 5′leader upstream of a therapeutic transgene has superior anti-tumour activity compared to a leaderless HSV1. This approach represented a simple yet highly effective way to improve the current generation of oncolytic HSV1 platforms that are presently in clinical trials. This strategy can be complementary to those that employ either a strong heterologous promoter to drive transgene expression, or an approach that inserts the transgene into a highly transcriptionally active region of the HSV1 genome. Although a secreted cytokine was used in this study, a membrane bound transgene can also be benefited from the expression enhancement effect of the 5′leader. The 5′leader can also be incorporated into multiple transgene expression cassettes, and inserted into multiple location within the HSV1 genome to simultaneously enhance the expression and therapeutic effect of each transgene. As an example of the former strategy, Toda et al. found that a GM-CSF expression cassette driven by a CMV promoter and inserted into the TK region yielded approximately 55 μg of GM-CSF per 10⁵ Vero cells. In the present study, which also employed a CMV-driven GM-CSF expression cassette inserted in the TK region of the HSV1 genome, the result observed was 41.75±2.35 μg GM-CSF per 10⁵ Vero cells, a value very close to that previously reported (FIG. 15A; calculated based on 167±9.4 μg/ml of GM-CSF observed in a 12-well plate format at confluency, which typically contains 4×10⁵ cells in 1 ml of culture media). Yet, with the introduction of the US11 5′leader, close to an 8-fold increase in GM-CSF production was obtained, representing a significant improvement in transgene protein expression. It is possible that transgene production could be improved even further by combining a strong HSV1 promoter (e.g., the one driving HSV1 RL2 expression) with a translation enhancer found in viral 5′ leaders and potentially also viral 3′UTRs. Additionally, minimal translation enhancing motif(s) specific for HSV1 infection could provide less recombination risk than full length 5′ leaders, especially if to be applied to novel generation of oncolytic HSV1 encoding for multiple therapeutic transgenes.

The present invention has been described with regard to one or more embodiments, however, it will be apparent to persons skilled in the art that a number of variations and modifications can be made without departing from the scope of the invention as defined by the claims.

All citations and/or references recited herein are hereby incorporated by reference in their entirety.

### REFERENCES

1. Rauch, S., Jasny, E., Schmidt, K. E. & Petsch, B. New vaccine technologies to combat outbreak situations. Front. Immunol. 9, 1963 (2018).
2. Macedo, N., Miller, D. M., Haq, R. & Kaufman, H. L. Clinical landscape of oncolytic virus research in 2020. Journal for ImmunoTherapy of Cancer 8, 1486 (2020).
3. Bulcha, J. T., Wang, Y., Ma, H., Tai, P. W. L. & Gao, G. Viral vector platforms within the gene therapy landscape. Signal Transduct. Target. Ther. 2021 61 6, 1-24 (2021).
4. Serafini, P. et al. High-dose granulocyte-macrophage colony-stimulating factor-producing vaccines impair the immune response through the recruitment of myeloid suppressor cells. Cancer Res. 64, 6337-6343 (2004).
5. Conner, J. & Braidwood, L. Expression of inhibitor of growth 4 by HSV1716 improves oncolytic potency and enhances efficacy. Cancer Gene Ther. 19, 499-507 (2012).
6. Pearl, T. M., Markert, J. M., Cassady, K. A. & Ghonime, M. G. Oncolytic Virus-Based Cytokine Expression to Improve Immune Activity in Brain and Solid Tumors. Molecular Therapy-Oncolytics 13, 14-21 (2019).
7. Zhang, Q. & Liu, F. Advances and potential pitfalls of oncolytic viruses expressing immunomodulatory transgene therapy for malignant gliomas. Cell Death and Disease 11, 1-11 (2020).
8. Zheng, M., Huang, J., Tong, A. & Yang, H. Oncolytic Viruses for Cancer Therapy: Barriers and Recent Advances. Molecular Therapy-Oncolytics 15, 234-247 (2019).
9. Cripe, T. P. et al. Pediatric cancer gone viral. Part I: Strategies for utilizing oncolytic herpes simplex virus-1 in children. Molecular Therapy-Oncolytics 2, 15015 (2015).
10. Walsh, D. & Mohr, I. Viral subversion of the host protein synthesis machinery. Nat Rev Microbiol 9, 860-875 (2011).
11. Hoang, H.-D., Graber, T. E. & Alain, T. Battling for Ribosomes: Translational Control at the Forefront of the Antiviral Response. J. Mol. Biol. (2018). doi: 10.1016/j.jmb.2018.04.040
12. Hu, J. C. C. et al. A phase I study of OncoVEXGM-CSF, a second-generation oncolytic herpes simplex virus expressing granulocyte macrophage colony-stimulating factor. Clin. Cancer Res. 12, 6737-6747 (2006).
13. Breitbach, C. J. et al. Intravenous delivery of a multi-mechanistic cancer-targeted oncolytic poxvirus in humans. Nat. 2011 4777362 477, 99-102 (2011).
14. Dhungel, P., Cao, S. & Yang, Z. The 5′-poly(A) leader of poxvirus mRNA confers a translational advantage that can be achieved in cells with impaired cap-dependent translation. PLoS Pathog. 13, e1006602 (2017).
15. Jha, S. et al. Trans-kingdom mimicry underlies ribosome customization by a poxvirus kinase. Nature 546, 651-655 (2017).
16. Mears, W. E. & Rice, S. A. The RGG box motif of the herpes simplex virus ICP27 protein mediates an RNA-binding activity and determines in vivo methylation. J. Virol. 70, (1996).
17. Sandri-Goldin, R. M. ICP27 mediates HSV RNA export by shuttling through a leucine-rich nuclear export signal and binding viral intronless RNAs through an RGG motif. Genes Dev. 12, 868-79 (1998).
18. Rivera, A. A. et al. Mode of transgene expression after fusion to early or late viral genes of a conditionally replicating adenovirus via an optimized internal ribosome entry site in vitro and in vivo. Virology 320, 121-134 (2004).
19. Bommareddy, P. K., Peters, C., Saha, D., Rabkin, S. D. & Kaufman, H. L. Oncolytic Herpes Simplex Viruses as a Paradigm for the Treatment of Cancer. Annu. Rev. Cancer Biol 2, 155-173 (2018).
20. Huang, C. J. & Wagner, E. K. The herpes simplex virus type 1 major capsid protein (VP5-UL19) promoter contains two cis-acting elements influencing late expression. J. Virol. 68, 5738-5747 (1994).
21. Steffy, K. R. & Weir, J. P. Upstream promoter elements of the herpes simplex virus type 1 glycoprotein H gene. J. Virol. 65, 972-975 (1991).
22. Pederson, N. E., Person, S. & Homa, F. L. Analysis of the gB promoter of herpes simplex virus type 1: high-level expression requires both an 89-base-pair promoter fragment and a nontranslated leader sequence. J. Virol. 66, (1992).
23. Goodart, S. A., Guzowski, J. F., Rice, M. K. & Wagner, E. K. Effect of genomic location on expression of beta-galactosidase mRNA controlled by the herpes simplex virus type 1 UL38 promoter. J. Virol. 66, 2973-2981 (1992).
24. Mavromara-Nazos, P. & Roizman, B. Delineation of regulatory domains of early (β) and late (γ2) genes by construction of chimeric genes expressed in herpes simplex virus 1 genomes. Proc. Natl. Acad. Sci. U.S.A 86, 4071-4075 (1989).
25. Rixon, F. J. & Clements, J. B. Detailed structural analysis of two spliced HSV-1 immediate-early mRNAs. Nucleic Acids Res. 10, 2241-2256 (1982).
26. Greco, A. et al. The DNA sequence coding for the 5′ untranslated region of herpes simplex virus type 1 ICP22 mRNA mediates a high level of gene expression. J. Gen. Virol. 75, 1693-1702 (1994).
27. Hoang, H. D. et al. Induction of an Alternative mRNA 5′ Leader Enhances Translation of the Ciliopathy Gene Inpp5e and Resistance to Oncolytic Virus Infection. Cell Rep. 29, 4010-4023.e5 (2019).
28. Tang, S., Patel, A. & Krause, P. R. Hidden regulation of herpes simplex virus 1 pre-mRNA splicing and polyadenylation by virally encoded immediate early gene ICP27. PLoS Pathog. 15, e1007884 (2019).
29. Tombacz, D. et al. Long-Read Isoform Sequencing Reveals a Hidden Complexity of the Transcriptional Landscape of Herpes Simplex Virus Type 1. Front. Microbiol. 8, 1079 (2017).
30. Tombacz, D. et al. Multiple Long-Read Sequencing Survey of Herpes Simplex Virus Dynamic Transcriptome. Front. Genet. 10, 834 (2019).
31. Whisnant, A. W. et al. Integrative functional genomics decodes herpes simplex virus 1. Nat. Commun. 11, 1-14 (2020).
32. Rutkowski, A. J. et al. Widespread disruption of host transcription termination in HSV-1 infection. Nat. Commun. 6, 7126 (2015).
33. Zhao, T. T. et al. hnRNP A1 regulates UV-induced NF-κB signalling through destabilization of clAP1 mRNA. Cell Death Differ. 16, 244-252 (2009).
34. Tang, S., Patel, A. & Krause, P. R. Herpes simplex virus ICP27 regulates alternative pre-mRNA polyadenylation and splicing in a sequence-dependent manner. Proc. Natl. Acad. Sci. U.S.A. 113, 12256-12561 (2016).
35. Minaker, R. L., Mossman, K. L. & Smiley, J. R. Functional inaccessibility of quiescent herpes simplex virus genomes. Virol. J. 2, (2005).
36. Donnelly, M. L. L. et al. Analysis of the aphthovirus 2A/2B polyprotein ‘cleavage’ mechanism indicates not a proteolytic reaction, but a novel translational effect: A putative ribosomal ‘skip’. J. Gen. Virol. 82, 1013-1025 (2001).
37. Kim, J. H. et al. High cleavage efficiency of a 2A peptide derived from porcine teschovirus-1 in human cell lines, zebrafish and mice. PLoS One 6, (2011).
38. Pol, J., Kroemer, G. & Galluzzi, L. First oncolytic virus approved for melanoma immunotherapy. Oncoimmunology 5, (2016).
39. Malhotra, S. et al. Use of an oncolytic virus secreting GM-CSF as combined oncolytic and immunotherapy for treatment of colorectal and hepatic adenocarcinomas. Surgery 141, 520-529 (2007).
40. Moesta, A. K. et al. Local delivery of OncoVEXmGM-CSF generates systemic antitumor immune responses enhanced by cytotoxic T-lymphocyte-associated protein blockade. Clin. Cancer Res. 23, 6190-6202 (2017).
41. Bhattacharya, P. et al. Dual Role of GM-CSF as a Pro-Inflammatory and a Regulatory Cytokine: Implications for Immune Therapy. J. Interf. Cytokine Res. 35, 585 (2015).
42. Kim, K. J. et al. Antitumor effects of IL-12 and GM-CSF co-expressed in an engineered oncolytic HSV-1. Gene Ther. 1-13 (2020). doi: 10.1038/s41434-020-00205-x
43. Toda, M., Martuza, R. L. & Rabkin, S. D. Tumor growth inhibition by intratumoral inoculation of defective herpes simplex virus vectors expressing granulocyte-Macrophage colony-stimulating factor. Mol. Ther. 2, 324-329 (2000).
44. Thomas, S. et al. Development of a new fusion-enhanced oncolytic immunotherapy platform based on herpes simplex virus type 1. J. Immunother. Cancer 7, 214 (2019).
45. Yan, W. L., Shen, K. Y., Tien, C. Y., Chen, Y. A. & Liu, S. J. Recent progress in GM-CSF-based cancer immunotherapy. Immunotherapy 9, 347-360 (2017).
46. Honess, R. W. & Roizman, B. Regulation of Herpesvirus Macromolecular Synthesis I. Cascade Regulation of the Synthesis of Three Groups of Viral Proteins 1. J. Virol. 14, 8-19 (1974).
47. Lee, K. M., Chen, C. J. & Shih, S. R. Regulation Mechanisms of Viral IRES-Driven Translation. Trends in Microbiology 25, 546-561 (2017).
48. Mailliot, J. & Martin, F. Viral internal ribosomal entry sites: four classes for one goal. Wiley Interdiscip. Rev. RNA 9, e1458 (2018).
49. Dauber, B., Saffran, H. A. & Smiley, J. R. The Herpes Simplex Virus 1 Virion Host Shutoff Protein Enhances Translation of Viral Late mRNAs by Preventing mRNA Overload. J. Virol. 88, 9624-9632 (2014).
50. Saffran, H. A., Read, G. S. & Smiley, J. R. Evidence for Translational Regulation by the Herpes Simplex Virus Virion Host Shutoff Protein. J. Virol. 84, 6041-6049 (2010).
51. Doepker, R. C., Hsu, W.-L., Saffran, H. A. & Smiley, J. R. Herpes Simplex Virus Virion Host Shutoff Protein Is Stimulated by Translation Initiation Factors elF4B and elF4H. J. Virol. 78, 4684-4699 (2004).
52. Feng, P., Everly, D. N. & Read, G. S. mRNA Decay during Herpes Simplex Virus (HSV) Infections: Protein-Protein Interactions Involving the HSV Virion Host Shutoff Protein and Translation Factors elF4H and elF4A. J. Virol. 79, 9651-9664 (2005).
53. Taddeo, B., Zhang, W. & Roizman, B. The virion-packaged endoribonuclease of herpes simplex virus 1 cleaves mRNA in polyribosomes. Proc. Natl. Acad. Sci. U.S.A 106, 12139-12144 (2009).
54. Taddeo, B., Sciortino, M. T., Zhang, W. & Roizman, B. Interaction of herpes simplex virus RNase with VP16 and VP22 is required for the accumulation of the protein but not for accumulation of mRNA. Proc. Natl. Acad. Sci. U.S.A 104, 12163-12168 (2007).
55. Corbin-Lickfett, K. A., Chen, I. H. B., Cocco, M. J. & Sandri-Goldin, R. M. The HSV-1 ICP27 RGG box specifically binds flexible, GC-rich sequences but not G-quartet structures. Nucleic Acids Res. 37, 7290-7301 (2009).
56. Chen, I.-H. B., Li, L., Silva, L. & Sandri-Goldin, R. M. ICP27 Recruits Aly/REF but Not TAP/NXF1 to Herpes Simplex Virus Type 1 Transcription Sites although TAP/NXF1 Is Required for ICP27 Export. J. Virol. 79, 3949-3961 (2005).
57. Koffa, M. D. et al. Herpes simplex virus ICP27 protein provides viral mRNAs with access to the cellular mRNA export pathway. EMBO J. 20, 5769-5778 (2001).
58. Schwer, B., Visca, P., Vos, J. C. & Stunnenberg, H. G. Discontinuous transcription or RNA processing of vaccinia virus late messengers results in a 5′ poly(A) leader. Cell 50, 163-169 (1987).
59. Shirokikh, N. E. & Spirin, A. S. Poly(A) leader of eukaryotic mRNA bypasses the dependence of translation on initiation factors. Proc. Natl. Acad. Sci. U.S.A 105, 10738-10743 (2008).
60. Jha, S. et al. Trans-kingdom mimicry underlies ribosome customization by a poxvirus kinase. Nature 546, 651-655 (2017).
61. Finkel, Y. et al. SARS-COV-2 utilizes a multipronged strategy to suppress host 2 protein synthesis Introduction. bioRxiv 2020.11.25.398578 (2020). doi: 10.1101/2020.11.25.398578
62. Kim, D., Langmead, B. & Salzberg, S. L. HISAT: A fast spliced aligner with low memory requirements. Nat. Methods 12, 357-360 (2015).
63. Afgan, E. et al. The Galaxy platform for accessible, reproducible and collaborative biomedical analyses: 2018 update. Nucleic Acids Res. 46, W537-W544 (2018).
64. Trapnell, C. et al. Transcript assembly and quantification by RNA-Seq reveals unannotated transcripts and isoform switching during cell differentiation. Nat. Biotechnol. 28, 511-515 (2010).
65. Graber, T. E., Baird, S. D., Kao, P. N., Mathews, M. B. & Holcik, M. NF45 functions as an IRES trans-acting factor that is required for translation of clAP1 during the unfolded protein response. Cell Death Differ. 17, 719-29 (2010).
66. Gandin, V. et al. Polysome fractionation and analysis of mammalian translatomes on a genome-wide scale. J. Vis. Exp. 1-9 (2014). doi: 10.3791/51455y.

What is claimed is:

1. A nucleic acid comprising:
a) SEQ ID NO: 1 or a fragment comprising at least 180 nucleotides thereof, or a sequence that is at least 90% identical to SEQ ID NO: 1;
wherein the nucleic acid does not comprise SEQ ID NO: 2, SEQ ID NO: 3, or both, and wherein, the nucleotide sequence does not comprise a fragment of SEQ ID NO: 2, 3 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 1; or
b) SEQ ID NO: 7 or a fragment comprising at least 180 nucleotides thereof, or a sequence that is at least 90% identical to SEQ ID NO: 7;
wherein the nucleic acid does not comprise SEQ ID NO: 8, SEQ ID NO: 9, or both, and wherein, the nucleotide sequence does not comprise a fragment of SEQ ID NO: 8, 9 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 7; or
c) SEQ ID NO: 13 or a fragment comprising at least 180 nucleotides thereof, or a sequence that is at least 90% identical to SEQ ID NO: 13;
wherein the nucleic acid does not comprise SEQ ID NO: 14, SEQ ID NO: 15, or both, and wherein, the nucleotide sequence does not comprise a fragment of SEQ ID NO: 14, 15 or both which is at least 1, at least 3, at least 5, at least 10, at least 15, at least 20, at least 25, at least 30, at least 35, at least 40, at least 45 or at least 50 nucleotide bases thereof immediately continuous with the 5′ or 3′ ends of SEQ ID NO: 13; or
d) the RNA counterpart of a); or
e) the RNA counterpart of b), or
f) the RNA counterpart of c).

2. The nucleotide sequence of claim 1, wherein the nucleic acid does not comprise at least 250, 500, 1000 or more continuous nucleotides of Human herpesvirus 1 strain KOS, complete genome defined by NCBI Accession number: JQ673480.1 GI: 380776962 or by Accession number JQ780693.1 GI: 384597744, or a sequence which is 95% identical thereto.

3. A second nucleic acid consisting of the nucleic acid of claim 1.

4. The nucleic acid of claim 1, which is a synthetic or recombinant nucleic acid.

5. The nucleic acid of claim 1, which is an expression vector or a plasmid.

6. The nucleic acid of claim 5, wherein the expression vector or plasmid drives production of a protein of interest, which is heterologous to HSV1.

7. The nucleic acid of claim 1, further comprising a promoter, a nucleotide sequence encoding a protein of interest, one or more regulatory sequences, one or more restriction endonuclease or cloning sites, one or more polyadenylation sites or any combination thereof, wherein at least one or more of the promoter, nucleotide sequence encoding a protein of interest, one or more restriction endonuclease or cloning sites, one or more polyadenylation sites, or any combination thereof is heterologous to HSV1.

8. The nucleic acid of claim 7, wherein the promoter is positioned upstream or 5′ to SEQ ID NO: 1, SEQ ID NO: 7, SEQ ID NO: 13 or the RNA counterpart thereof.

9. The nucleic acid of claim 7, wherein the nucleotide sequence encoding a protein of interest is positioned downstream or 3′ to SEQ ID NO: 1, SEQ ID NO: 7, SEQ ID NO: 13 or the RNA counterpart thereof.

10. The nucleic acid of claim 7, wherein the promoter is positioned immediately upstream to SEQ ID NO: 1, SEQ ID NO: 7, SEQ ID NO: 13 or the RNA counterpart thereof and the nucleotide sequence encoding a protein of interest is positioned immediately downstream to SEQ ID NO: 1, SEQ ID NO: 7, SEQ ID NO: 13 or the RNA counterpart thereof.

11. The nucleic acid of any one of claims 1-10, wherein the nucleotide sequence is circular or linear.

12. An RNA nucleic acid defined by the counterpart of the nucleic acid of claim 1.

13. The nucleic acid of claim 7, wherein SEQ ID NO: 1, SEQ ID NO: 7, SEQ ID NO: 13 or the RNA counterpart thereof, increases translation of the nucleotide sequence encoding a protein of interest in a cell compared to its translation in the absence of SEQ ID NO: 1, SEQ ID NO: 7, SEQ ID NO: 13 or the RNA counterpart thereof wherein the increased translation occurs only when the cell is pre-infected with HSV1 virus.

14. The nucleic acid of claim 7, wherein the protein of interest is a reporter protein, a cell regulatory protein or a cytotoxic protein.

15. A vector comprising:
a) SEQ ID NO: 1 or a fragment comprising at least 180 nucleotides thereof, or a sequence that is at least 90% identical to SEQ ID NO: 1; or
b) SEQ ID NO: 7 or a fragment comprising at least 180 nucleotides thereof, or a sequence that is at least 90% identical to SEQ ID NO: 7; or
c) SEQ ID NO: 13 or a fragment comprising at least 180 nucleotides thereof, or a sequence that is at least 90% identical to SEQ ID NO: 13; or
d) the RNA counterpart of a); or
e) the RNA counterpart of b), or
f) the RNA counterpart of c).
and;
a promoter, a nucleotide sequence encoding a protein of interest, one or more regulatory sequences, one or more restriction endonuclease or cloning sites, and one or more polyadenylation sites or any combination thereof.

16. The vector of claim 15, wherein the vector is a viral vector recombinantly transformed with a heterologous nucleic acid comprising:
a) SEQ ID NO: 1 or a fragment comprising at least 180 nucleotides thereof, or a sequence that is at least 90% identical to SEQ ID NO: 1;
b) SEQ ID NO: 7 or a fragment comprising at least 180 nucleotides thereof, or a sequence that is at least 90% identical to SEQ ID NO: 7;
c) SEQ ID NO: 13 or a fragment comprising at least 180 nucleotides thereof, or a sequence that is at least 90% identical to SEQ ID NO: 13;
d) the RNA counterpart of a);
e) the RNA counterpart of b), or
f) the RNA counterpart of c).
and;
a promoter, a nucleotide sequence encoding a protein of interest, one or more regulatory sequences, one or more restriction endonuclease or cloning sites, and one or more polyadenylation sites or any combination thereof.

17. The viral vector of claim 16, wherein at least one of SEQ ID NO:1, SEQ ID NO: 7, SEQ ID NO: 13, fragment thereof or RNA counterpart thereof, the sequence that is at least 90% identical to SEQ ID NO: 1, SEQ ID NO: 7, SEQ ID NO: 13 or a the fragment thereof comprising at least 180 nucleotides, the promoter, the nucleotide sequence encoding a protein of interest, the one or more regulatory sequences, the one or more restriction endonuclease or cloning sites, the one or more polyadenylation sites, or any combination thereof is heterologous to the viral vector.

18. The viral vector of claim 16, wherein the vector is a live, attenuated, oncolytic or any combination thereof.

19. The viral vector of claim 16, which is an HSV1 viral vector.

20. The viral vector of claim 19, wherein the HSV1 viral vector is HSV1.

21. A cell comprising the nucleic acid of any one of claims 1-14, or the vector of any one of claims 15-20.

22. The cell of claim 21, wherein the cell is a mammalian cell infected by HSV1 virus.

23. The cell of claim 22, wherein the mammalian cell is a cancer cell infected by HSV1 virus.

24. The cell of claim 21, wherein the cell exhibits an increase in protein expression, synthesis, or production when infected by HSV1 virus of more than about 0.5 fold, about 1 fold, about 2 fold, about 3 fold, about 4 fold, about 5 fold, about 6 fold, about 7 fold, about 8 fold or more compared to an identical control cell which lacks the nucleic acid.

25. The nucleic acid of any one of claims 1-14 or the vector of any one of claims 15-20, or the cell of claim 21-24, wherein the nucleic acid is inserted anywhere in HSV1.

26. The nucleic acid of any one of claims 1-14 or the vector of any one of claims 15-20, or the cell of claim 21-24, wherein the nucleic acid is inserted at any restriction site or at the tk-locus of HSV1.

27. A composition comprising:
the nucleic acid of any one of the claims 1-14, the vector of any one of claims 15-20, or the cell of any one of claims 21-24 alone or in combination, and;
optionally comprising one or more pharmaceutically acceptable carriers, excipients, or diluents.

28. A kit comprising:
a) the nucleic acid of any one of the claims 1-14,
b) the vector of any one of claims 15-20,
c) the cell of any one of claims 21-24,
d) one or more pharmaceutically acceptable carriers, excipients, or diluents,
e) one or more buffers, wash or cell culture media,
f) one or more vessels for containing a)-e),
g) instructions for expressing or augmenting expression of a protein of interest;
h) instructions for using any component of the kit,
or any combination of a)-h).

29. A method of producing a protein of interest in a cell comprising:
administering a nucleic acid to the cell which is pre-infected with HSV1 virus, the nucleic acid comprising a) a promoter, b) SEQ ID NO: 1, SEQ ID NO: 7, SEQ ID NO: 13 or its RNA counterpart, and c) a sequence encoding a protein of interest which is expressed from the nucleic acid in the cell.

30. A method of increasing expression, synthesis, or production of a protein of interest in a cell comprising:
administering a nucleic acid to the cell which is pre-infected with HSV1 virus, the nucleic acid comprising a) a promoter; b) SEQ ID NO: 1, SEQ ID NO: 7, SEQ ID NO: 13 or its RNA counterpart thereof; and c) a sequence encoding a protein of interest which is expressed from the nucleic acid in the cell, wherein the increase in expression, synthesis or production of a protein of interest is relative to a similar step of administering a nucleic acid in the absence of SEQ ID NO: 1; SEQ ID NO: 7, SEQ ID NO: 13 or its RNA counterpart.

31. The method of claim 30, wherein the promoter is positioned upstream or 5′ to SEQ ID NO: 1, SEQ ID NO: 7, SEQ ID NO: 13 or its RNA counterpart and the sequence encoding a protein of interest is positioned downstream or 3′ to SEQ ID NO: 1, SEQ ID NO: 7, SEQ ID NO: 13 or its RNA counterpart.

32. The method of claim 30, wherein the promoter is positioned immediately upstream to SEQ ID NO: 1, SEQ ID NO: 7, SEQ ID NO: 13 or its RNA counterpart and the sequence encoding a protein of interest is positioned immediately downstream to SEQ ID NO: 1, SEQ ID NO: 7, SEQ ID NO: 13 or its RNA counterpart.

33. The method of claim 30, wherein the nucleic acid is present in HSV1 and the cell is infected with HSV1 virus.

34. The method of claim 30, wherein the nucleic acid is in a plasmid, or a vector.

35. The method of claim 34, wherein the vector is selected from a viral vector, a live-viral vector, an oncolytic viral vector, an attenuated viral vector, a recombinant vector or an amplicon vector.

36. A method of improving or treating a medical condition, cellular defect or disease in a subject comprising:
administering a nucleic acid to the cells of the subject in need thereof, wherein the cells are pre-infected with HSV1 virus; the nucleic acid comprising: a) a promoter, b) SEQ ID NO: 1, SEQ ID NO: 7, SEQ ID NO: 13 or its RNA counterpart, and c) a sequence encoding a protein of interest which is expressed from the nucleic acid in the cells, wherein the protein of interest improves or treats the medical condition, cellular defect or disease in the subject;
wherein the expression, synthesis, or production of the protein of interest in the cells of the subject improves or treats the medical condition, cellular defect or disease in the subject.

37. The method of claim 36, wherein the promoter is positioned upstream or 5′ to SEQ ID NO: 1, SEQ ID NO: 7, SEQ ID NO: 13 or its RNA counterpart and the sequence encoding a protein of interest is positioned downstream or 3′ to SEQ ID NO: 1, SEQ ID NO: 7, SEQ ID NO: 13 or its RNA counterpart.

38. The method of claim 36, wherein the promoter is positioned immediately upstream to SEQ ID NO: 1, SEQ ID NO: 7, SEQ ID NO: 13 or its RNA counterpart and the sequence encoding a protein of interest is positioned immediately downstream to SEQ ID NO: 1, SEQ ID NO: 7, SEQ ID NO: 13 or its RNA counterpart.

39. The method of claim 36, wherein the nucleic acid in in HSV1.

40. The method of claim 39, wherein the HSV1 is a live-viral vector, an oncolytic viral vector, or an attenuated viral vector.

41. A method of treating a cancer in a subject comprising:
administering a nucleic acid to the cells of the patient in need thereof, wherein the cells are pre-infected with HSV1 virus, the nucleic acid comprising a) a promoter, b) SEQ ID NO: 1, SEQ ID NO: 7, SEQ ID NO: 13 or its RNA counterpart and a sequence encoding a protein of interest which is expressed from the nucleic acid in the cells, wherein the protein of interest is capable of treating cancer in the subject;
wherein the expression, synthesis, or production of the protein of interest in the cells of the patient treats the cancer condition.

42. The method of claim 41, wherein the cancer is melanoma.

43. A method of improving the efficacy of an existing gene therapy comprising:
modifying a gene delivery vehicle being used in the existing gene therapy by inserting a sequence of SEQ ID NO: 1, SEQ ID NO: 7, SEQ ID NO: 13 or its RNA counterpart, wherein the gene delivery vehicle comprises a sequence encoding a protein of interest;
administering the modified gene delivery vehicle to the patient in need thereof; wherein the patient is pre-infected with HSV1 virus;
wherein transcription of SEQ ID NO: 1, SEQ ID NO: 7, SEQ ID NO: 13 or its RNA counterpart increases translation of the sequence encoding the protein of interest causing an increase in expression, production or synthesis of the protein of interest relative to its expression, production or synthesis in the absence of SEQ ID NO: 1, SEQ ID NO: 7, SEQ ID NO: 13 or its RNA counterpart, thereby improving the efficacy of the gene therapy.

44. The method of claim 43, wherein the increase in expression, production or synthesis of the protein of interest is by several fold compared to its expression, production or synthesis in the absence of SEQ ID NO: 1, SEQ ID NO: 7, SEQ ID NO: 13 or its RNA counterpart.

45. The method of claim 44, wherein the increase in expression, production or synthesis of the protein of interest is 0.5 fold, 1 fold, 2 fold, 3 fold, 4 fold, 5 fold, 6 fold, 7 fold, 8 fold or more.

46. The method of claim 43, wherein the existing gene therapy is an oncolytic viral therapy.

47. The method of claim 43, wherein the existing gene therapy is a gene based immunotherapy.

48. A method of increasing transgene expression in a cell:
administering a nucleic acid to the cell, wherein the cell is pre-infected with HSV1 virus the nucleic acid comprising a) a promoter, b) SEQ ID NO: 1, SEQ ID NO: 7, SEQ ID NO: 13 or its RNA counterpart and c) a transgene which is expressed from the nucleic acid in the cell, wherein the transgene encodes a protein of interest;
wherein the increase in expression of the transgene is relative to a similar step of administering a nucleic acid in the absence of SEQ ID NO: 1, SEQ ID NO: 7, SEQ ID NO: 13 or its RNA counterpart.
